(19) United States
(12) Patent Application Publication
(10) Pub. No.: US 2012/0190064 A1
(43) Pub. Date: Jul. 26, 2012

(54) FEEDING BUFFERS, SYSTEMS, AND METHODS FOR IN VITRO SYNTHESIS OF BIOMOLECULES

(75) Inventors: Wieslaw Antoni KUDLICKI, Carlsbad, CA (US); Shiranthi Keppetipola, Carlsbad, CA (US); Julia Fletcher, Carlsbad, CA (US); Ashley Elaine Getbehead, Oceanside, CA (US); Federico Katzen, San Marcos, CA (US); Laura Vozza-Brown, Carlsbad, CA (US)

(73) Assignee: LIFE TECHNOLOGIES CORPORATION, Carlsbad, CA (US)

(21) Appl. No.: 13/356,368

(22) Filed: Jan. 23, 2012

Related U.S. Application Data
(63) Continuation of application No. 12/211,094, filed on Sep. 15, 2008, now abandoned.
Application No. 12/211,094 is a continuation of application No. 11/240,651, filed on Oct. 3, 2005, now abandoned.
(60) Provisional application No. 60/720,450, filed on Sep. 27, 2005.
(60) Provisional application No. 60/656,534, filed on Feb. 28, 2005.
(60) Provisional application No. 60/642,094, filed on Jan. 10, 2005.
(60) Provisional application No. 60/614,590, filed on Oct. 1, 2004.

(51) Int. Cl. C12P 21/02; C12N 9/12

(52) U.S. Cl. 435/68.1; 435/194

(57) ABSTRACT

Compositions, methods and kits for in vitro systems for synthesis of biomolecules such as polypeptides, are provided herein. Cell extracts that provide enhanced yields of soluble proteins using in vitro protein synthesis methods are provided. The invention also includes methods for producing high yields of proteins by the addition of a feeding solution that includes amino acids and an energy source to an ongoing in vitro synthesis system. The invention also includes methods of using a high-yield in vitro synthesis system to produce large quantities of proteins with incorporated labeled amino acids for analysis by methods such as by NMR. The invention further includes vectors for enhanced production of proteins from nucleic acid templates using in vitro synthesis systems.

### RELATED APPLICATIONS

This application claims the benefit of, and incorporates by reference, U.S. provisional patent applications Ser. No. 60/614,590, filed Oct. 1, 2004, Ser. No. 60/642,094, filed Jan. 10, 2005, Ser. No. 60/656,534, filed Feb. 28, 2005, and Ser. No. ______, having attorney docket number 0942.6660003, filed Sep. 27, 2005, all of which name Kudlicki et al. as inventors, and all of which are entitled “FEEDING BUFFERS, SYSTEMS, AND METHODS FOR IN VITRO SYNTHESIS OF BIOMOLECULES.”

### FIELD OF THE INVENTION

This invention relates to the field of biotechnology. In particular, the invention relates to in vitro systems for synthesizing, purifying, labeling and/or detecting biomolecules, such as nucleic acids and polypeptides.

### BACKGROUND OF THE INVENTION

In vitro protein synthesis (IVPS) has among its advantages specifically producing the desired protein without unnecessarily producing undesired proteins that are required for maintaining cells used for protein production in in vivo or cellular systems for protein synthesis. When a cell is used as a protein factory, in addition to producing the desired protein, the cell produces the other necessary molecules, including undesired proteins, which are required to maintain the cell.

Because it is essentially free from cellular regulation of gene expression, in vitro protein synthesis has advantages in the production of cytotoxic, unstable, or insoluble proteins. The over-production of protein beyond a predetermined concentration can be difficult to obtain in vivo, because the expression levels are regulated by the concentration of product. The concentration of protein accumulated in the cell generally affects the viability of the cell, so that over-production of the desired protein is difficult to obtain. In an isolation and purification process, many kinds of protein are insoluble or unstable, and are either degraded by intracellular proteases or aggregate in inclusion bodies, so that the loss rate is high. In vitro synthesis circumvents many of these problems. Moreover, through simultaneous and rapid expression of various proteins in a multiplexed configuration, this technology can provide a valuable tool for development of combinatorial arrays for research, and for screening of proteins. In addition, various kinds of unnatural amino acids can be efficiently incorporated into proteins for specific purposes (Noren et al, Science 244: 182-188, 1989). However, despite all its promising aspects, the in vitro system has not been widely accepted as a practical alternative for in vivo synthesis of proteins.

Existing E. coli based cell-free expression systems offer a number of advantages when compared with cell-based systems, including speed to results and ease of use. These systems are becoming increasingly used, particularly in the field of Proteomics. See Movahedzadeh et al., In vitro transcription and translation. Methods Mol Biol. 235:247-255 (2003); Kim et al., Eur. J. Biochem. 239:881, 1996; Patnaik and Swartz, Biotechniques 24:862, 1998; Kim and Swartz (1999) Biotechnol. and Bioeng. 66:180-188; and Kim and Swartz (2000) Biotechnol. Prog. 16:385-390. The availability of complete genome sequences provides a wealth of information on the molecular structure and organization of a myriad of genes and open reading frames whose functions are not known or are only poorly understood. Thus, the utility of IVTT and more generally, protein synthesis in vitro, is expected to be even more important in the future for rapid and efficient protein synthesis and functional analysis.

However, current IVPS systems have their limitations. For example, these systems do not produce sufficient quantities of protein for extensive analysis. It is difficult to produce a desirable amount (mg) of the protein of interest, at a desirable concentration (e.g., mg/ml), in a short period of time (1-6 hours).

Moreover, methods for the highly efficient incorporation of unnatural (e.g., detectably labeled) amino acids into a protein of interest in an IVPS are limited. Mamaev et al. (Anal Biochem 326: 25-32, 2004) have reported a method for incorporating labeled amino acids into a protein during IVPS, but only at the amino terminus and only through the addition of an exogenous initiator suppressor tRNA chemically aminoacylated with a fluorophore-amino acid conjugate. Moreover, the methods of Mamaev et al. achieve only 27-67% specific labeling.

Patents in the field of in vitro protein synthesis include without limitation those in the following list. This listing is not intended to be a comprehensive review of the relevant art, nor is the listing of any of these patent documents an admission that any of the documents are, in fact, relevant art.

U.S. Pat. No. 5,478,730, to Alakhov et al., entitled “Method of preparing polypeptides in cell-free translation system.”

U.S. Pat. Nos. 5,665,563; 5,492,817; and 5,324,637, to Beckler et al., entitled

“Coupled transcription and translation in eukaryotic cell-free extract.”

U.S. Pat. No. 6,337,191 to Swartz et al., entitled “In vitro Protein Synthesis using Glycolytic Intermediates as an Energy Source.”

U.S. Pat. No. 6,518,058 to Biryukov et al., “Method of preparing polypeptides in cell-free system and device for its realization.”

U.S. Pat. No. 6,670,173, to Schels et al., entitled “Bioreaction module for biochemical reactions.”

U.S. Pat. No. 6,783,957 to Biryukov et al., entitled “Method for synthesis of polypeptides in cell-free systems.”

United States Patent Application 2002/0168706 to Chatterjee et al., published Nov. 14, 2002, entitled “Improved in vitro synthesis system.”

U.S. Pat. No. 6,168,931 to Swartz et al., issued Jan 8, 2002, entitled “In vitro macromolecule biosynthesis methods using exogenous amino acids and a novel ATP regeneration system.”

U.S. Pat. No. 6,548,276 to Swartz et al., issued Apr. 15, 2003, entitled “Enhanced in vitro synthesis of active proteins containing disulfide bonds.”

United States Patent Application 2004/0110135 to Nemetz et al., published Jun. 10, 2004, entitled “Method for producing linear DNA fragments for the in vitro expression of proteins.”

United States Patent Application 2004/0209321 to Swartz et al., published Oct. 21, 2004, entitled “Methods of in vitro protein synthesis.”

United States Patent Application 2004/0214292 to Motoda et al., published Oct. 28, 2004, entitled “Method of producing template DNA and method of producing protein in cell-free protein synthesis system using the same.”

United States Patent Application 2004/0259081 to Watzele et al., published Dec. 23, 2004, entitled “Method for protein expression starting from stabilized linear short DNA in cell-free in vitro transcription/translation systems with exonuclease-containing lysates or in a cellular system containing exonucleases.”

United States Patent Applications 2005/0009013, published Jan. 13, 2005, and 2005/0032078, published Feb. 10, 2005, both to Rothschild et al. and both entitled “Methods for the detection, analysis and isolation of nascent proteins.”

United States Patent Application 2005/0032086 to Sakanyan et al., published Feb. 10, 2005, entitled “Methods of RNA and protein synthesis.”

Published PCT patent application WO 00/55353 to Swartz et al., published Mar. 15, 2000, entitled “In vitro macromolecule biosyntheis methods using exogenous amino acids and a novel ATP regeneration system.”

All of these patents and patent applications are hereby incorporated by reference in their entireties.

### SUMMARY OF THE INVENTION

The invention is drawn to the in vitro synthesis of biomolecules, such as in vitro protein synthesis (IVPS). The invention provides compositions, methods, cloning and expression vectors, and kits for IVPS.

The present invention relates to compositions, methods and kits for in vitro protein synthesis (IVPS). The invention includes IVPS systems, as well as compositions, methods and kits thereof. Also, two or more different elements (compositions, methods, kits) can be combined in different aspects of the invention. The methods of the present invention are useful for making compositions for IVPS systems and for efficiently carrying out IVPS reactions. The compositions of the present invention are used to produce proteins of interest, and can be derived from any biological source (e.g., viruses, cells or organelles from a prokaryote, a eukaryote, an archea, an animal, a plant, a bacterium, etc.).

The invention provides a Feeding Solution (also referred to herein as a Feeding Buffer) that comprises some components of the IVPS reaction, and that is added after the IVPS reaction has been initiated. A Feeding Solution can be added to an ongoing cell-free expression reaction to extend protein synthesis and generate higher yields. The present invention provides potent Feeding Solutions having many desirable features (e.g., greater yield of protein, shorter reaction times, and the like).

A Feeding Solution according to the invention comprises at least one additional energy source and/or co-factor. By “additional”, it is meant that the energy source and/or co-factor are structurally different from the energy source(s) and/or co-factor(s) found exclusively or predominantly in the original reaction mixture. Typically, the original energy sources in the reaction at time 0 (t=0) are phosphoenol-pyruvate (PEP) and acetyl phosphate (AP). Preferred additional energy sources to be included in the Feeding Solution (and/or added to the initial IVPS reaction) include without limitation glycolytic intermediates such as Glucose-6-Phospate, Fructose-6-phosphate, or 3-Phosphoglycerate, with the cofactors NAD or NADH.

The invention also provides cell extracts that produce increased yields of soluble protein in an IVPS system. The extracts are made by adding a lipid, surfactant, or detergent to the buffer in which the cells are lysed to produce the extract.

The invention further provides vectors for efficient cloning of protein coding sequences, in which the vectors have sequences that promote translation, solubility, and activity of the protein encoded by the sequences.

In another aspect, the invention is drawn to IVPS methods, including without limitation the use of one or more Feeding Solutions, IVPS cell extracts, and/or vectors, including kitted versions thereof, that maximize protein synthesis in terms of yield and time. The invention provides methods that synthesize milligram quantities of a protein of interest (POI), preferably at a concentration from at least 1 to about 1 mg/ml to 100 or about 100 mg/ml, in from about 1 to about 6 hours.

In another aspect, the invention is drawn to IVPS methods and compositions, including without limitation one or more Feeding Solutions, IVPS extracts and/or vectors, and kitted versions thereof, that maximize the incorporation of exogenously added amino acids during the IVPS reaction. Such exogenously added amino acids can include detectably labeled amino acids, such as fluorescently labeled amino acids, heavy isotope amino acids, and radiolabeled amino acids. These aspects of the invention are useful for labeling desired proteins for methods such as mass spectrometry and nuclear magnetic resonance (NMR) spectroscopy, as they provide for complete incorporation of detectably labeled or other unnatural amino acids to the exclusion of the corresponding unlabeled (natural) amino acids. The invention also provides systems and kits that can be used to achieve complete labeling of proteins useful for mass spectroscopy and NMR spectroscopy.

In another aspect, the invention is drawn to vectors for cloning and expressing a gene of interest in an IVPS system. A preferred feature of in vitro protein synthesis is that it is a defined system into which a gene of interest can be introduced to direct the production of a specified protein of interest. The efficiency of expression of a gene of interest is influenced by sequences outside of its reading frame, and desired regulatory sequences can be operably linked to a gene of interest in a vector according to the invention. A set of two vectors is provided in which the vectors allow fusion of a protein of interest to an amino acid tag sequence, in which one of the two vectors can be used to express a protein of interest having an N-terminal amino acid tag, and the other of the two vectors can be used to express a protein of interest having an C-terminal amino acid tag. The vectors provide for efficient production of a desired protein that can be isolated, affinity purified, or detected using the amino acid tag, where the synthesized protein has a minimum of added amino acids.

### BRIEF DESCRIPTION OF THE DRAWINGS

FIG. 1 shows an exemplary timeline of in vitro protein synthesis (IVPS) utilizing Feeding Solutions.

FIG. 2 shows real-time expression of green fluorescent protein (GFP).

FIG. 3 shows production of milligram amounts of different proteins using an. IVPS system of the present invention.

FIG. 4 shows the effects of the using detergent in preparation of the cell extract used for IVPS on the amount of soluble protein synthesized.

FIG. 5 shows the effects of the using detergent in preparation of the cell extract.

FIG. 6 shows the effects of the using detergent in preparation of the cell extract.

FIG. 7 shows the effects of adding detergent extracts of the S30 pellet to IVPS reactions on the amount of protein synthesized.

FIG. 8 shows the sequences of cloning cassettes of several expression vectors of the invention underlined sequence under “RBS”, ribosome binding site; double-underlined text: TOPO® sequence (5′-CCCTT); underlined sequence under “ATG” text, start codon; and open reading frame (ORF).

FIG. 9 shows the pEXP5 NT/TOPO® vector. (A) vector map; (B and C) nucleotide and amino acid sequence encoded by pEXP5-NT/TOPO®. The arrow indicates the TEV cleavage site between the glutamine (“Q”) and serine (“S”) residues. Only two additional amino acid residues, serine and leucine (“L”) remain in the main polypeptide chain after TEV cleavage. Abbreviation: GOI, gene of interest.

FIG. 10 shows a method of TOPO® cloning using the pEXP5-NT/TOPO® vector.

FIG. 11 shows the pEXP5-CT/TOPO® vector. (A) vector map; (B) nucleotide and amino acid sequences of the cloning cassette, including the amino sequence (KGHHHHHH) generated when no stop codon is present in the GOI.

FIG. 12 shows a method of TOPO® cloning using the pEXP5-CT/TOPO® vector.

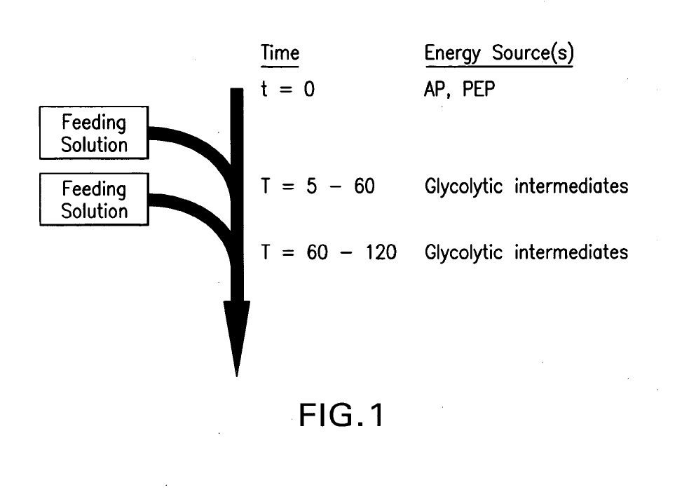

### ABBREVIATIONS AND DEFINITIONS

In the description that follows, a number of terms used in recombinant nucleic acid technology are utilized extensively. In order to provide a clear and more consistent understanding of the specification and claims, including the scope to be given such terms, the following definitions are provided.

Amino Acids: As used herein, an “amino acid” is an organic compound containing an amino group (—NH₂) and a carboxyl group (—COOH).

The following table describes the set of 20 naturally occurring amino acids commonly found in natural proteins and the one and three letter codes associated with each amino acid:

TABLE 1
NATURALLY OCCURRING AMINO ACIDS
AND THE GENETIC CODE

| | 3-LETTER | 1-LETTER | |
|---|---|---|---|
| FULL NAME | CODE | CODE | STANDARD CODONS* |
| Alanine | Ala | A | GCU, GCC, GCA, GCG |
| Arginine | Arg | R | CGU, CGC, CGA, CGG, |
| | | | AGA, AGG |
| Asparagine | Asn | N | AAU, AAC |
| Aspartic Acid | Asp | D | GAU, GAC |
| Cysteine | Cys | C | UGU, UGC |
| Glutamine | Gln | Q | CAA, CAG |
| Glutamic Acid | Glu | E | GAA, GAG |
| Glycine | Gly | G | CGU, CGC, CGA, CGG |
| Histidine | His | H | CAU, CAC |
| Isoleucine | Ile | I | AUU, AUC, AUA |
| Leucine | Leu | L | UUA, UUG, CUU, CUC, |
| | | | CUA, CUG |
| Lysine | Lys | K | AAA, AAG |
| Methionine | Met | M | AUG |
| Phenylalanine | Phe | F | UUU, UUC |
| Proline | Pro | P | CCU, CCC, CCA, CCG |
| Serine | Ser | S | UCU, UCC, UCA, UCG, |
| | | | AGU, AGC |
| Threonine | Thr | T | ACU, ACC, ACA, ACG |
| Tryptophan | Trp | W | UGG |
| Tyrosine | Tyr | Y | UAU, UAC |
| Valine | Val | V | GUU, GUC, GUA, GUG |

*Codons are depicted in this table as they appear in mRNA. Corresponding codons in DNA molecules would substitute a thymidine (T) nucleotide for any uracil (U) nucleotide in the RNA sequence.

The term “extra amino acids” is used herein to refer to amino acids that are not present in a natural protein, but which are introduced into a protein that is expressed using recombinant DNA. As used herein, “natural” and “wildtype” refer to a biological molecule as it occurs in nature. The terms “unnatural” and “modified” refer to a biological molecule that has modified or altered relative to its natural form.

The term “amino acids” thus encompasses both natural amino acids (Table 1) and modified amino acids. Modified amino acids include without limitation detectably labeled and/or structurally modified amino acids. Naturally, for protein synthesis, preferred modified amino acids are those that can be incorporated into a polypeptide during translation.

Arsenical molecule: As used herein, an arsenical molecule is any chemical compound comprising one or more atoms of Arsenic. Preferred arsenical molecules bind a specific amino acid sequence. A preferred specific amino acid sequence is C-C-X-X-C-C, wherein “C” represents cysteine and “X” represents any amino acid other than cysteine. Both biarsenical (2 arsenic atoms) and tetraarsenical (4 arsenic atoms) compounds are arsenical compounds. A tetraarsenical molecule is both an arsenical and biarsenical molecule.

An arsenical, biarsenical or tetraarsenical molecule preferably includes a detectable group, for example a fluorescent group, a luminescent group, a phosphorescent group, a spin label, a photosensitizer, a photocleavable moiety, a chelating center, a heavy atom, a radioactive isotope, an isotope detectable by nuclear magnetic resonance (NMR), a paramagnetic atom, and combinations thereof. For some applications, the biarsenical molecule is immobilized on a solid phase, preferably by covalent coupling. Such applications include being immobilized on beads or some other substrate suitable for affinity chromatography. This is used to purify tagged proteins. An arsenical, biarsenical or tetraarsenical molecule preferably is capable of traversing a biological membrane.

Biarsenical molecule: As used herein a biarsenical molecule is any chemical compound comprising two or more atoms of Arsenic. Preferred biarsenical molecules bind a specific amino acid sequence. A preferred specific amino acid sequence is C-C-X-X-C-C, wherein “C” represents cysteine and “X” represents any amino acid other than cysteine. See U.S. Patent Application Publication No.2005017065, herein incorporated by reference for all disclosure regarding biarsenical molecules.

Tetraarsenical molecule: Other molecules that can used instead of or in combination with a biarsenical molecule include without limitation a tetraarsenical molecule. The tetraarsenical molecule includes two biarsenical molecules having chemical formulas disclosed in U.S. Pat. No. 6,054,271 to Tsien, herein incorporated by reference for all disclosure regarding tetraarsenical molecules. For example, two biarsenical molecules are coupled to each other through a linking group.

Cellular extract or cell extract: An extract is a cell lysate or exudate, or a fraction thereof. For example, a cell extract can be a portion of a lysate from which other cellular components of the lysate have been separated by centrifugation, filtration, selective precipitation, selective immunoprecipitation, chromatography, or other methods. For example, commonly practiced methods of making a cell extract for IVPS include centrifuging a cell lysate to pellet membranes and other insoluble components of the lysate and remove the supernatant, which is the extract to be used in the IVPS system. The terms “cell extract” and “IVPS extract” also encompass mixtures of components crafted to imitate a cell lysate or exudate with respect to the components necessary or desired for protein or nucleic acid synthesis. An IVPS extract thus can be a mixture of components to imitate or improve upon a cell lysate or exudate (or fraction thereof) in protein synthesis reactions and/or to provide components used for synthesis from a nucleic acid template. Such mixtures, as will be recognized by one of ordinary skill in the art, can be produced by obtaining a partial extract or fraction thereof and/or by mixing any number of individual components. The latter can be from a natural source or be synthesized in vitro.

Depleted: As used herein, the term “depleted” indicates the lack of a given substance or component. Herein, a composition is deplete of a substance if that substance is present at a concentration that is, by v/v, w/v or w/w, less than 1% or about 1%, preferably less than 0.1% or about 0.1%, most preferably less than 0.01% or about 0.01% of the composition.

Substantially depleted: Herein, a composition is substantially depleted of a substance if it is most preferably less than 25% or about 25%, preferably less than 10% or about 10%, most preferably less than 5% or about 5%, most preferably less than 1.1% or about 1.1%,

Detectably labeled: The terms “detectably labeled” and “labeled” are used interchangeably herein and are intended to refer to situations in which a molecule (e.g., a nucleic acid molecule, protein, nucleotide, amino acid, and the like) have been tagged with another moiety or molecule that produces a signal capable of being detected by any number of detection means, such as by instrumentation, eye, photography, radiography, and the like. In such situations, molecules can be tagged (or “labeled”) with the molecule or moiety producing the signal (the “label” or “detectable label”) by any number of art-known methods, including covalent or ionic coupling, aggregation, affinity coupling (including, e.g., using primary and/or secondary antibodies, either or both of which may comprise a detectable label), and the like. Suitable detectable labels for use in preparing labeled or detectably labeled molecules in accordance with the invention include, for example, radioactive isotope labels, fluorescent labels, chemiluminescent labels, bioluminescent labels and enzyme labels, and others that will be familiar to those of ordinary skill in the art.

Gene: As used herein, the term “gene” refers to a nucleic acid that contains information necessary for expression of a polypeptide, protein, or untranslated RNA (e.g., rRNA, tRNA, anti-sense RNA). When the gene encodes a protein, it includes the promoter and the structural gene open reading frame sequence (ORF), as well as other sequences involved in expression of the protein. When the gene encodes an untranslated RNA, it includes the promoter and the nucleic acid that encodes the untranslated RNA.

Gene of interest (GOI): The term “gene of interest” refers to any nucleotide sequence (e.g., RNA or DNA), the manipulation of which may be deemed desirable for any reason (e.g., treat disease, confer improved qualities, expression of a protein of interest in a host cell, expression of a ribozyme, etc.), by one of ordinary skill in the art. Such nucleotide sequences include, but are not limited to, coding sequences of structural genes (e.g., reporter genes, selection marker genes, oncogenes, drug resistance genes, growth factors, etc.), and non-coding regulatory sequences which do not encode an mRNA or protein product (e.g., promoter sequence, polyadenylation sequence, termination sequence, enhancer sequence, etc.).

Host: As used herein, the term “host” refers to any prokaryotic or eukaryotic (e.g., mammalian, insect, yeast, plant, avian, animal, etc.) organism that is a recipient of a replicable expression vector, cloning vector or any nucleic acid molecule. The nucleic acid molecule may contain, but is not limited to, a sequence of interest, a transcriptional regulatory sequence (such as a promoter, enhancer, repressor, and the like) and/or an origin of replication. As used herein, the terms “host,” “host cell,” “recombinant host” and “recombinant host cell” may be used interchangeably. For examples of such hosts, see Sambrook, et al., Molecular Cloning: A Laboratory Manual, Cold Spring Harbor Laboratory, Cold Spring Harbor, N.Y.

In vitro: As used herein, “in vitro” refers to systems outside a cell or organism and may sometimes be referred to cell free system. In vivo systems relate to essentially intact cells whether in suspension or attached to or in contact with other cells or a solid. In vitro systems have an advantage of being more able to be manipulated. Delivering components to a cell interior is not a concern; manipulations incompatible with continued cell function are also possible. However, in vitro systems involve disrupted cells or the use of various components to provide the desired function and thus spatial relationships of the cell are lost. When an in vitro system is prepared, components, possibly critical to the desired activity can be lost with discarded cell debris. Thus in vitro systems are more manipulatable and can function differently from in vivo systems.

IVT: The terms “in vitro transcription” (IVT) and “cell-free transcription” are used interchangeably herein and are intended to refer to any method for cell-free synthesis of RNA from DNA without synthesis of protein from the RNA. A preferred RNA is messenger RNA (mRNA), which encodes proteins.

IVTT: The terms “in vitro transcription-translation” (IVTT), “cell-free transcription-translation”, “DNA template-driven in vitro protein synthesis” and “DNA template-driven cell-free protein synthesis” are used interchangeably herein and are intended to refer to any method for cell-free synthesis of mRNA from DNA (transcription) and of protein from mRNA (translation).

IVPS: The terms “in vitro protein synthesis” (IVPS), “in vitro translation”, “cell-free translation”, “RNA template-driven in vitro protein synthesis”, “RNA template-driven cell-free protein synthesis” and “cell-free protein synthesis” are used interchangeably herein and are intended to refer to any method for cell-free synthesis of a protein. IVTT, including coupled transcription and transcription, is one non-limiting example of IVPS.

IVPS-competent: As used herein, the terms “IVPS-competent” and “competent for IVPS” refer to an IVPS extract or system that can be used to produce a polypeptide in vitro.

Kitted: As used herein, the term “kilted” is used to indicate compositions that have been prepared in the form of a kit. A kit is a collection of compositions that can include one or more reagents, one or more devices, or one or more supplies, where two or more of the compositions of the kit can be used in the same or different steps of a protocol or method. Optionally, the compositions can be conveniently provided together, such as in a box, rack, crate, package, etc., in one or more individual containers, such as tubes, vials, bubble packs, blister packs, etc., preferably along with written instructions that directly or indirectly provide a user with instructions for use. In some cases, however, one or more components of a kit can be packaged separately.

Nucleic Acid Molecule: As used herein, the phrase “nucleic acid molecule” refers to a sequence of contiguous nucleotides (riboNTPs, dNTPs, ddNTPs, or combinations thereof) of any length. A nucleic acid molecule may encode a full-length polypeptide or a fragment of any length thereof, or may be non-coding. As used herein, the terms “nucleic acid molecule” and “polynucleotide” may be used interchangeably and include both single-stranded (ss) and double-stranded (ds) RNA, DNA and RNA:DNA hybrids.

Polymerase: As used herein, a “polymerase” is an enzyme that catalyses synthesis of nucleic acids using a preexisting nucleic acid template. Examples include DNA polymerase (which catalyzes DNA→DNA reactions), RNA polymerase (DNA→RNA) and reverse transcriptase (RNA→DNA).

Polypeptide: As used herein, the term “polypeptide” refers to a sequence of contiguous amino acids of any length. The terms “peptide,” “oligopeptide,” or “protein” may be used interchangeably herein with the term “polypeptide.”

Promoter: As used herein, the terms “promoter,” “promoter element,” or “promoter sequence” refer to a DNA sequence which when ligated to a nucleotide sequence of interest is capable of controlling the transcription of the nucleotide sequence of interest into mRNA. A promoter is typically, though not necessarily, located 5′ (i.e., upstream) of a nucleotide sequence of interest whose transcription into mRNA it controls, and provides a site for specific binding by RNA polymerase and other transcription factors for initiation of transcription. Promoters may be constitutive or regulatable. The term “constitutive” when made in reference to a promoter means that the promoter is capable of directing transcription of an operably linked nucleic acid sequence in the absence of a stimulus (e.g., heat shock, chemicals, etc.). In contrast, a “regulatable” promoter is one that is capable of directing a level of transcription of an operably linked nucleic acid sequence in the presence of a stimulus (e.g., heat shock, chemicals, etc.), which is different from the level of transcription of the operably linked nucleic acid sequence in the absence of the stimulus

Protein of Interest (POI): As used herein, the terms protein of interest, POI, and “desired protein” refer to a polypeptide under study, or whose expression is desired by one practicing the methods disclosed herein. A protein of interest is encoded by its cognate gene of interest (GOI). The identity of a POI can be known or not known. A POI can be a polypeptide encoded by an open reading frame.

Solubilizing agent: As used herein, the terms “solubilizing agent” and “solubilizer” refer to any compound that helps a second, hydrophobic compound remain or go into solution in a solvent, typically water.

Transcription: As used herein, unless otherwise stated, the term “transcription” refers to the synthesis of RNA from a DNA template.

Translation: As used herein, unless otherwise stated, the term “translation” refers to the synthesis of a polypeptide from an mRNA template.

Vector: As used herein, the term “vector” refers to any genetic element, such as a plasmid, phage, transposon, cosmid, chromosome, virus, virion, etc., which is capable of replication when associated with the proper control elements and which can transfer gene sequences between cells. The vector may contain a marker suitable for use in the identification of transformed cells. For example, markers may provide tetracycline resistance or ampicillin resistance. Types of vectors include cloning and expression vectors.

Vector, Cloning: As used herein, the term “cloning vector” refers to a plasmid or phage DNA or other DNA sequence which is able to replicate autonomously in a host cell and which is characterized by one or a small number of restriction endonuclease recognition sites and/or sites for site-specific recombination. A foreign DNA fragment may be spliced into the vector at these sites in order to bring about the replication and cloning of the fragment

Vector, Expression: As used herein, the term “expression vector” refers to a vector which is capable of expressing of a gene that has been cloned into it. Such expression can occur after transformation into a host cell, or in IVPS systems. The coned DNA is usually operably linked to one or more regulatory sequences, such as promoters, repressor binding sites, terminators, enhancers and the like. The promoter sequences can be constitutive, inducible and/or repressible.

Other terms used in the fields of recombinant nucleic acid technology and molecular and cell biology as used herein will be generally understood by one of ordinary skill in the applicable arts.

### DETAILED DESCRIPTION OF THE INVENTION

In vitro Protein Synthesis (IVPS)

In General

Cell extracts have been developed that support the synthesis of proteins in vitro from purified mRNA transcripts or from mRNA transcribed from DNA during the in vitro synthesis reaction. Such protein synthesis systems are called “IVPS systems” herein, IVPS being an acronym for “In vitro Protein Synthesis.”

The general system includes a nucleic acid template that encodes a protein of interest. The nucleic acid template is an RNA molecule (e.g., mRNA) or a nucleic acid that encodes an mRNA (e.g., RNA, DNA) and be in any form (e.g., linear, circular, supercoiled, single stranded, double stranded, etc.). Nucleic acid templates guide production of the desired protein. IVPS systems can also be engineered to guide the incorporation of detectably labeled amino acids, or unconventional or unnatural amino acids, into a desired protein.

In a generic IVPS reaction, a gene encoding a protein of interest is expressed in a Transcription Buffer, resulting in mRNA that is translated into the protein of interest in an IVPS extract and a Translation Buffer. The Transcription Buffer, IVPS extract and Translation Buffer can be added separately, or two or more of these solutions can be combined before their addition, or added contemporaneously.

To synthesize a protein of interest in vitro, an IVPS extract must at some point comprise a mRNA molecule that encodes the protein of interest. In early IVPS experiments, mRNA was added exogenously after being purified from natural sources or prepared synthetically in vitro from cloned DNA using bacteriophage RNA polymerases. In other systems, the mRNA is produced in vitro from a template DNA; both transcription and translation occur in this type of IVPS reaction. Techniques using coupled or complementary transcription and translation systems, which carry out the synthesis of both RNA and protein in the same reaction, have been developed. In such in vitro transcription and translation (IVTT) systems, the IVPS extracts contain all the components necessary both for transcription (to produce mRNA) and for translation (to synthesize protein) in a single system. An early IVTT system was based on a bacterial extract (Lederman and Zubay, Biochim. Biophys. Acta, 149: 253, 1967). In IVTT systems, the input nucleic acid is DNA, which is normally much easier to obtain than mRNA, and more readily manipulated (e.g., by cloning, site-specific recombination, and the like).

Regardless of how they are prepared, or in which order they are added, an IVTT reaction mixture comprises the following components:

a template nucleic acid, such as DNA, that comprises a gene of interest (GOI) operably linked to at least one promoter and, optionally, one or more other regulatory sequences (e.g., a cloning or expression vector containing the GOI);

an RNA polymerase that recognizes the promoter(s) to which the GOI is operably linked and, optionally, one or more transcription factors directed to an optional regulatory sequence to which the template nucleic acid is operably linked;

ribonucleotide triphosphates (rNTPs);

optionally, other transcription factors and co-factors therefor;

ribosomes;

transfer RNA (tRNA);

other or optional translation factors (e.g., translation initiation, elongation and termination factors) and co-factors therefore;

amino acids (optionally comprising one or more detectably labeled amino acids);

one or more energy sources, (e.g., ATP, GTP);

optionally, one or more energy regenerating components (e.g., PEP/pyruvate kinase, AP/acetate kinase or creatine phosphate/creatine kinase);

optionally factors that enhance yield and/or efficiency (e.g., nucleases, nuclease inhibitors, protein stabilizers) and co-factors therefore; and

optionally, solubilizing agents.

Components of IVPS reactions are discussed in more detail below.

### Template Nucleic Acids and RNA Polymerases

A nucleic acid template is a polynucleic acid that serves to direct synthesis of another nucleic acid template or of a protein. The template is a molecule composed of numerous nucleotide subunits, but can vary in length and in the type of nucleotide subunits. DNA and RNA, e.g., mRNA, are species of nucleic acids that can be used as templates for protein and nucleic acid synthesis. A DNA template is transcribed to form an RNA template complementary to all or a portion of said template. An RNA template is translated to produce a protein or peptide encoded by all or a portion of the template. Thus, the template in a synthesis reaction is one or more species of nucleic acid that codes directly or indirectly for desired protein(s).

When the template is a DNA template, an RNA molecule must be transcribed by a RNA polymerase before protein can be synthesized. RNA polymerases suitable for use in the present methods include any polymerase that is active in the chosen system with the chosen template to synthesize protein. The IVPS cellular extract may contain a suitable polymerase, such as RNA polymerase II, SP6 RNA polymerase, T3 RNA polymerase, T7 RNA polymerase, RNA polymerase III and/or phage derived RNA polymerases. These and other polymerases are known in the art and can be readily assessed by the skilled artisan by searching one or more of the public or private databases. Suitable polymerase can also be supplemented in the system. A RNA polymerase that recognizes the promoter to which the desired gene is operably linked is used. RNA polymerases and transcription factors useful in the invention are known in the art and will be readily recognized by those skilled in the art.

Modulation of RNA polymerases can be helpful in IVPS systems that use a DNA template to produce RNA. When RNA synthesis is rapid, the RNA may be insufficiently protected by ribosomes. Use of a mutated or modulated RNA polymerase can advantageously spare the RNA by allowing ribosomes proper time to bind and protect the nascent RNA.

Optionally, the template nucleic acid may have additional regulatory sequences for optional transcription regulatory factors including without limitation repressors, activators, transcription and translation enhancers, DNA-binding proteins, and the like.

Transfer RNA (tRNA)

Typically, the tRNA molecules present in an IVPS extract are derived from the source cells used to prepare the extract. However, the invention provides IVPS extracts that are depleted in endogenous tRNA. The tRNA-depleted IVPS reaction can be controlled by the addition of tRNA molecules, which can be synthetic or derived from another biological source. In addition, mutant tRNAs can be used to incorporate unnatural amino acids into proteins for specific purposes.

Charged tRNA molecules are also within the scope of tRNA molecules that can be used in the invention. A charged tRNA (a.k.a. an aminoacyl-tRNA) comprises a specific tRNA and a specific amino acid covalently attached to the 3′OH of the tRNA.

### Amino Acids

Typically, at least some of the amino acids present in an IVPS extract are derived from the source cells used to prepare the extract. However, the invention provides IVPS extracts that are substantially depleted in endogenous amino acids. The IVPS reaction can then be controlled by the addition of amino acids, which can be synthetic or derived from another biological source.

As a non-limiting example of amino acids derived from a biological source that is different than the source of the IVPS, algal amino acid mixtures can be used. These may lack certain amino acids, particularly Asn, Cys, Gln and Trp, a characteristic that can be used in various ways in NMR studies. Unlabeled algal amino acid extract can be used in combination with supplements of the amino acids Asn, Cys, Gln and/or Trp, which can be unlabeled or labeled (with, by way of non-limiting example, ²H, ¹³C and/or ¹⁵N). Labeled forms of specific amino acids (Asn, Cys, Gln and/or Trp) can thus be used to specifically label only certain amino acid residues in a protein. Algal amino acid mixtures are commercially available in both labeled and un-labeled form (Cambridge Isotope Laboratories, Andover, Mass.; Sigma-Aldrich, St. Louis, Mo.).

Various kinds of unnatural amino acids, including without limitation detectably labeled amino acids, can be added to IVPS reactions and efficiently incorporated into proteins for specific purposes. See, for example, Noren et al., Science 244:182-188 (1989); Anthony-Cahill et al., Trends Biochem Sci. 14:400-403 (1989); Ellman et al., Methods Enzymol. 202:301-336 (1991); and Liu et al., Proc. Natl. Acad. Sci. USA 94:10092-10097 (1997).

### Energy Sources and Energy Regenerating Components

Protein and nucleic acid synthesis typically requires an energy source. It is thus a feature of the present invention to provide a sufficient energy source to support such synthesis. Energy is required for initiation of transcription to produce mRNA (e.g., when a DNA template is used and for initiation of translation high energy phosphate for example in the form of GTP is used). Each subsequent step of one codon by the ribosome (three nucleotides; one amino acid) requires hydrolysis of an additional GTP to GDP. ATP is also typically required. For an amino acid to be polymerized during protein synthesis, it must first be activated. Activation requires hydrolysis of two high-energy phosphate bonds. Thus an amino acid monomer in the presence of Mg⁺² tRNA and ATP reacts to form an aminoacyl-tRNA, AMP and inorganic pyrophosphate (PPᵢ). Significant quantities of energy from high energy phosphate bonds are thus required for protein and/or nucleic acid synthesis to proceed.

An energy source is a chemical substrate that can be enzymatically processed to provide energy to achieve desired chemical reactions. Energy sources that allow release of energy for synthesis by cleavage of high energy phosphate bonds such as those found in nucleoside triphosphates, e.g., ATP, are commonly used. Other energy sources, for example sources that can form high-energy phosphate bonds, can also power the synthesis process. Exemplary energy sources for use in in vitro synthesis are glucose, phosphoenolpyruvate (PEP), carbamoyl phosphate, acetyl phosphate, creatine phosphate, phosphopyruvate, glyceraldehyde-3-phosphate, pyruvate, 3-Phosphoglycerate, fructose-6-phosphate, and glucose-6-phosphate. Any source convertible to high energy phosphate bonds is especially suitable. For example, pyruvate kinase catalyzes a reaction of PEP and ADP to form pyruvate and ATP. ATP can be reversibly converted to triphosphates of the other ribonucleosides. Thus ATP, GTP, and other triphosphates can normally be considered as equivalent energy sources for supporting protein synthesis.

To provide energy for the synthesis reaction, the system preferably includes added energy sources, such as glucose, pyruvate, phosphoenolpyruvate (PEP), carbamoyl phosphate, acetyl phosphate, creatine phosphate, phosphopyruvate, glyceraldehyde-3-phosphate, 3-Phosphoglycerate and glucose-6-phosphate, that can generate or regenerate high-energy triphosphate compounds such as ATP, GTP, other NTPs, etc. The energy source can be present in any amount that is suitable for the desired synthesis. For example, the chemical energy source can be added to achieve a concentration of from 10-100 mM. About 15, 20, 25, 30, 50, 60, 70, 80 or 90 mM may also be target concentrations. The precise concentration will vary as synthesis consumes energy and the energy is replenished from these sources. The concentration for a particular energy source molecule may be controlled within various ranges, for example about 10-100 mM, 15-90 mM, 20-80 mM, 30-60 mM, etc. Any target concentration can be used as an approximate boundary for the desired range of concentration of energy source. When two or more energy source molecules are used, each source can independently be one of these or another concentration.

When sufficient energy is not initially present in the synthesis system, an additional source of energy is preferably supplemented. The supplement can be delivered continuously or can be delivered in one or more discreet supplements. One feature of the present invention includes addition of at least two (or three or more, four or more, five or more, six or more, etc.) energy sources to provide the energy for the synthesis reactions of the invention. At least one of the supplemented energy sources can be provided to the extract prior to setting up the reaction for in vitro synthesis of a protein of interest. For example, one or more of an energy providing enzyme, a glycolytic intermediate, or another energy source molecule can be provided to the extract at a time point prior to the initiation of the IVPS reaction. In addition, least one, and preferably at least two, energy sources can be provided at the outset of the reaction for in vitro synthesis of a protein of interest. In particular, glycolytic intermediates are used in the invention as supplemental energy sources and include without limitation glucose-6-phosphate (G-6-P), fructose-6-phosphate (F-6-P), and 3-phosphoglycerate. PEP, AP and the cofactors NAD or NADH can also be added.

Energy sources can also be added or supplemented during the in vitro synthesis reaction. When multiple energy sources are included in the system, synthesis (especially protein synthesis) is found to be accelerated and prolonged in time, so that protein and/or nucleic acid products are more efficiently produced by the synthesis system. For example, when phosphoenol pyruvate (PEP) and acetyl phosphate are used as initial energy sources, the amount of protein synthesized can be more than doubled as compared to when only acetyl phosphate is added. Thus, the present invention includes an in vitro synthesis system that comprises at least two, and preferably at least three different energy sources that provide high energy phosphate bonds for the synthesis reactions, where the energy sources can be substrate molecules of enzymes.

The use of the specified combinations of energy sources and cofactors (Glucose-6-Phosphate and NADH) has been described for use in cell-free expression reactions as the primary energy supply for the reaction. See U.S. Pat. No. 6,337,191 to Swartz et al., entitled “In vitro Protein Synthesis using Glycolytic Intermediates as an Energy Source” and U.S. Pat. No. 6,168,931 to Swartz et al., entitled “Enhanced in vitro Synthesis of Biological Macromolecules Using a Novel ATP Regeneration System” both of which are incorporated by reference herein for all disclosure relating to energy sources and energy regenerating systems, including enzymes and substrates.

Published PCT patent application WO 00/55353 discloses two methods for replenishing ATP necessary for translation. According to these methods, PEP (phosphoenolpyruvate) or pyruvate is used to regenerate the ATP energy source. The first disclosed method was previously known in the art and involves phosphoenolpyruvate (PEP) used in conjunction with pyruvate kinase to regenerate ATP from ADP. In the second method of WO 00/55353, pyruvate is used in conjunction with pyruvate oxidase to regenerate ATP. Both energy sources and amino acids are depleted in these systems irrespective of protein synthesis (WO 00/55353; Kim and Choi, J. Biotech., 84:27, 2000).

### Nucleases and Nuclease Inhibitors

Maintenance of the template is desirable to maximize the duration of the synthesis process. The synthesis system of the present invention can include components that maintain the template. The template can be maintained by preventing enzymatic, chemical or other degradation of the template. The synthesis system of the invention therefore can include modifications to the extract to improve product synthesis. When the extract contains enzymes whose activities compromise protein and/or nucleic acid production, inhibition of these enzymes will result in more efficient synthesis by the system. Thus, in vitro synthesis systems comprising inhibitors of at least one enzyme are embodiments of the present invention. Nuclease and phosphatase inhibitors are advantageously used to increase protein and/or nucleic acid synthesis efficiency. Inhibition of enzymes that unnecessarily consume compounds used in the synthesis reaction can also improve synthesis efficiency. Depending on the specific enzymes present in the extract, for example, one or more of the many known nuclease, polymerase or phosphatase inhibitors can be selected and advantageously used to improve synthesis efficiency.

To maintain the template, cells that are used to produce the extract can be selected for reduction, substantial reduction or elimination of activities of detrimental enzymes or for enzymes with modified activity. Thus, in vitro synthesis systems comprising extracts of cells having altered activity (for example by modifying or mutating one or more genes) are embodiments of the present invention. Cells with modified nuclease or phosphatase activity (e.g., with at least one mutated phosphatase or nuclease gene or combinations thereof) are especially advantageously used for synthesis of cell extracts to increase synthesis efficiency. For example, an E. coli strain used to make an S30 extract for IVPS can be RNase E or RNase A deficient (for example, by mutation).

Examples of nucleases that can be removed, inhibited, mutated, modified, or modulated include without limitation: exonuclease I, exonuclease II, exonuclease III, DNA polymerase II, DNA polymerase III (ε subunit), exonucleases WA and IVB, RecBCD (exonuclease V), exonuclease VII, exonuclease VIII, RecJ, dRpase, endonuclease I, endonuclease III, endonuclease IV, endonuclease V, endonuclease VII, endonuclease VIII, fpg, uvrABC, mutH, vsr endonuclease, ruvC, ecoK, ecoB, mcrBC, mcrA, mrr, and TOPO®isomerases (such as TOPO®isomerase I, TOPO®isomerase II, TOPO®isomerase III and TOPO®isomerase IV). Such removal, inhibition, etc., allows preservation or protection of the nucleic acid template used in the synthesis reactions of the invention. For example, DNA nucleases of cells can be mutated, modified, inhibited, etc. to maintain or preserve the DNA templates. Such DNases from E. coli and other cells are known in the art.

Modulation of RNA nucleases may also be helpful in IVPS systems that use a DNA template to produce RNA. When RNA synthesis is rapid, the RNA may be insufficiently protected by ribosomes. RNA nucleases can be mutated, modified, inhibited, etc. to protect or preserve the RNA template. For example, E. coli ribonucleases, such as endoribonuclease I, M, R, III, P, E, K, H, HII, IV, F, N, P2, 0, PC and PIV, and exonucleases such as polynucleotide phosphorylase, oligoribonuclease, and exoribonucleases II, D, BN, T, PH and R, can be mutated or modified or inhibited to protect mRNA for protein synthesis. Depending on the cell used for the extract, other ribonucleases native to that cell can be mutated, removed, modified, or inhibited, etc. to maintain or protect the template(s) for protein synthesis. For example, an E. coli strain used to make an extract for IVPS can have a mutation that reduces or eliminates RNase E activity. U.S. Patent Application Publication 2002/0168706 is hereby incorporated by reference for all disclosure related to the use of cell extracts having reduced activity of a nuclease in IVPS systems. Many nucleases and nuclease inhibitors are commercially available. For example, RNasin® (Promega), is well characterized as an RNase inhibitor in mammalian systems, but is not effective in inhibiting prokaryotic Rnases.

In addition, inhibitors, such as inhibitors of nucleases that act on nucleic acid templates, particularly linear templates such as linear DNA templates (e.g., Gam protein of phage lambda to inhibit RecBCD) or inhibitors of other unwanted or detrimental components/proteins/enzymes in the synthesis reaction can be used to enhance the production of desired products in vitro. Inhibitors can be used or included in the systems of the invention by any known method. For example, inhibitors may be added to the system before, during or after introduction of the nucleic acid template. Inhibitors can also be transcribed or expressed in a cell used to prepare the extract or transcribed or expressed during the protein synthesis reaction. Although inhibitors may be biosynthetic compounds, inhibitors of the invention are not limited to compounds that can be produced biologically. U.S. Patent Application Publication 2002/0168706 is hereby incorporated by reference for all disclosure related to the use of cell extracts having inhibitors of nucleases in IVPS systems.

### Solubilizing Agents

Agents that help solubilize IVPS components and/or polypeptide products can be added to IVPS systems. In some aspects of the invention, one or more lipids, surfactants, or detergents is added to an IVPS extract, IVPS reaction or Feeding Solution as a solubilizing agent or for another purpose. One or more lipids, one or more surfactants, or one or more detergents, or any combinations thereof, can be added to an IVPS reaction to improve the protein yield, the soluble protein yield, or the active protein yield of the system. Without wishing to be limited to any particular mechanism, lipids, surfactants, and/or detergents can improve the solubility of proteins or of components of the IVPS extract. Preferred lipids includes phospholipids, disclosed elsewhere herein. Surfactants can include any surfactants, including, but in no way limited to, nondetergent sulfobetaine surfactants. Preferred detergents are nonionic and zwitterionic detergents, further described elsewhere herein.

In some aspects of the present invention, nanoscale phospholipid bilayer discs can be included in the IVPS reaction mixture. Such phospholipid-protein particles or “nanodiscs” that include phospholipids in a bilayer structure engirdled by a scaffold protein such as Apolipoprotein A1 (Apo A1) or derivatives thereof, have been described by Bayburt et al. (J. Struct. Biol. 123: 37-44 (1998)) and Bayburt and Sligar (PNAS 99:6725-6730 (2002); Protein Science 12:2476-2481 (2004)) and are disclosed in U.S. Patent Application Publication No. 2005/0182243, herein incorporated by reference for all disclosure related to nanoscopic phospholipid bilayer discs and their component phospholipids and scaffold proteins. The inclusion of nanoscopic phospholipid bilayer discs can improve the yield of soluble protein, particularly when membrane proteins are synthesized in IVPS reactions. For example, nanoscopic phospholipid bilayer discs can be included in an IVPS reaction, such as those described herein, at a concentration of from 0.1 to 100 mm, preferably from 0.2 to 50 m, and more preferably yet from 0.5 mm to 40 mm. For example, nanodiscs can be present in an IVPS reaction at from about 1 mm to about 20 mm.

When nanoscopic phospholipid bilayer discs are included in an IVPS reaction, the solubility of in vitro translated membrane proteins is greatly increased. When nanoscopic phospholipid bilayer discs are included in an IVPS reaction, the in vitro translated membrane proteins are inserted into the nanoscopic phospholipid bilayer discs, and can be isolated in soluble form integrated within the nanodiscs using affinity tags provided on the scaffold protein of the nanodiscs.

### Preparation of Ivps Extracts

Typically, several components (e.g., ribosomes, trna, translation factors, and co-factors therefore) of an ivps reaction are provided in the form of an ivps extract that is prepared from a biological source. Any type of biological source, including without limitation prokaryotic cells, eukaryotic cells, organelles and viruses can be used as a biological source for an ivps system (see, e.g., pelham et al, European Journal Of Biochemistry, 67:247, 1976). Prokaryotic systems benefit from simultaneous or “coupled” transcription and translation.

Eukaryotic IVPS systems include without limitation rabbit reticulocyte lysates, wheat germ lysates, Drosophila embryo extracts, scallop lysates (Storch et al. J. Comparative Physiology B, 173:611-620, 2003), extracts from mouse brain (Campagnoni et al., J Neurochem. 28:589-596, 1977; Gilbert et al. J Neurochem. 23:811-818, 1974), and chick brain (Liu et al. Transactions of the Illinois State Academy of Science, Volume 68, 1975).

The extract can be prepared by any method used in the art that maintains the integrity of the transcription/translation system or, if the process damages one or more component necessary for any stage of transcription/translation, the damaged component can be replaced or substituted for after the extract preparation. Bacterial extracts can be prepared according to the method of Zubay (1973) and modifications thereof. The ordinarily skilled artisan will recognize that many modifications to the extraction process are possible within the scope of the present invention. The extract preferably includes all necessary components for synthesis that are not otherwise provided in the system. Enzymes and other components present in the extract to provide energy and other components for the synthesis reaction can originate in the extracted cell or can be added during the production of the extract. The extract can be supplemented to add or increase the concentration of components not present, or not present in sufficient or optimal quantities, respectively. The extract can also be concentrated using one or more of the many tools of the art.

In a typical method for preparing an IVPS extract, the extract is processed to remove cellular debris. Centrifugation is a common method for removing such solid material. Filtration, chromatography, or any other separation or purification procedures may be used to produce a desired extract. In some cases, undesirable components of an extract can be removed, for example by using affinity reagents that can capture or remove one or more undesirable components.

In some aspects of the invention, an IVPS extract is prepared from a mutant organism or cell. In particular, IVPS extracts can be prepared from cells lacking, or having reduced levels of, the SlyD protein. This is particularly desirable when it is intended to use the IVPS extract for producing fusion proteins comprising a sequence of six consecutive Histidine residues (“His tag”) and/or a amino acid sequence that binds a detectably labeled arsenical molecule (“FlAsH or LUMIO tag). SlyD interacts with both of these amino acid sequences and is thus a frequent contaminant of fusion proteins produced in wildtype bacteria or in IVPS extracts therefrom. U.S Patent Application Publication No. US2005/0136449 is hereby incorporated by reference for all disclosure relating to the use of cell extracts in translation systems that have reduced levels of the SlyD protein.

### Expressway Ivps Systems

In some embodiments, the invention relates to, or uses as an assay, one or more Expressway™ IVPS systems (Invitrogen, Carlsbad, Calif.). Expressway™ systems include without limitation the following:

The Expressway™ Plus Expression System utilizes a coupled transcription and translation reaction to produce active recombinant protein. The Expressway™ Plus System provides all the components for cell-free protein production. The kit includes an E. coli extract containing the cellular machinery required to drive transcription and translation. The IVPS Plus reaction buffer is also included in the kit and contains the required amino acids (except methionine) and an ATP regenerating system for energy. The reaction buffer, methionine, T7 Enzyme Mix, and DNA template of interest, operably linked to a T7 promoter, are mixed with the E. coli extract. As the DNA template is transcribed, the 5′ end of the mRNA is bound by ribosomes and undergoes translation as the 3′ end of the template is still being transcribed.

The Expressway™ Linear Expression System is used for rapid high-yield in vitro expression from linear DNA templates. The system uses an E. coli extract optimized for expression of full-length, active protein from linear templates. As a result, linear templates are more stable during transcription and translation, resulting in higher yields of properly folded products. In the Expressway™ Linear Expression System, at least two options are available for generating T7 promoter-driven templates. The Expressway™ Linear Expression Kit can be used to express PCR templates generated from a plasmid containing the appropriate elements for expression (T7 promoter, ribosome binding site, T7 termination sequence). The Expressway™ Linear Expression Kit with TOPO® Tools includes a 5′ and 3′ element that can be operably joined to a PCR product. The 5′ element contains a T7 promoter, ribosome binding site, and start codon. The 3′ element contains a V5 epitope tag followed by a 6×His region and a T7 terminator. The TOPO® Tools elements are joined to the PCR product in a TOPO® ligation reaction and then amplified by PCR.

The Expressway™ Plus Expression System with Lumio™ Technology Kit includes IVPS Lumio™ E. coli Extract, IVPS Plus E. coli Reaction Buffer, RNase A, T7 Enzyme Mix, Methionine, reaction tubes, pEXP3-DEST vector, a control plasmid, and a Lumio™ Green Detection Kit or components thereof. See Keppetipola et al., Rapid Detection of in vitro expressed proteins using Lumio™ Technology. Focus 25.3:7, 2003.

These and other Expressway™ systems are described in detail in the following Manufacturer's Instruction Manuals for these products, all of which are incorporated by reference herein for disclosure of IVPS systems and methods:
Expressway™ In vitro Protein Synthesis System Manual, Version C, Apr. 11, 2003 (see the worldwide web at www.invitrogen.com/content/sfs/manuals/expressway_man.pdf);
Expressway™ Linear Expression System Manual, Version A, 26 Sep. 2003 (see the worldwide web at www.invitrogen.com/content/sfs/manuals/expresswaylinear_man.pdf);
Expressway™ Linear Expression System with TOPO® Tools Technology, Version A, 26 Sep. 2003 (see the worldwide web at www.invitrogen.com/content/sfs/manuals/expresswaylinearwithTOPO®tools_man.pd f)
Expressway Plus Expression System Manual, Version A, 26 Sep. 2003 (see the worldwide web at www.invitrogen.com/content/sfs/manuals/expresswayplus_man.pdf); and
Expressway Plus Expression System with Lumio Technology Manual, Version B, 27 Feb. 2004 (see the worldwide web at www.invitrogen.com/content/sfs/manuals/expresswayplus_lumio_man.pdf).

### Feeding Solutions

In some aspects the invention is drawn to a feeding solution and methods of making and using a feeding solution. A feeding solution is a solution added to an in vitro protein synthesis (IVPS) reaction after the reaction has been initiated. A feeding solution therefore does not supply an essential component of the IVPS, in that reaction proceeds in the absence of the feeding solution. A feeding solution is added while the IVPS reaction is ongoing, and enhances one or more of the yield of protein, the yield of soluble protein, or the yield of active protein made by the system.

By way of background, IVPS systems generally involve four types of IVPS reactions.

1. Batch IVPS Reaction: In a Batch Reaction, there is fast initial rate of synthesis that slows and eventually stops after about 3 hours. The composition of the reaction mix changes as amino acids are incorporated or metabolized, and energy sources are metabolized, generating inhibitory free phosphate. See, e.g., Kawarsaki et al., Anal. Biochem. 226:320, 1995; Patnaik et al., BioTechniques 24:862, 1998; and Kigawa et al., J. Biochem., 110:166, 1991.

2. Feeding/Dilution IVPS Reaction: IVPS reactions can be prolonged by supplying fresh components over time through a “feeding solution” (aka “feeding buffer”), which may also have the desirable effect of diluting inhibitory by-products. On the other hand, however, extensive dilution of transcription and/or translation factors may cause a decrease in or loss of the activity of the IVPS system.

3. Bilayer Overlay IVPS Reaction: The more dense reaction mix is overlayed with a feeding solution, and components are exchanged through passive diffusion. The reaction rate is slower due to “non-shaking” of the reaction vessel. See, e.g., Sawasaki et al., 2 FEBS Lett 514:102, 2002.

4. Continuous Exchange IVPS Reaction: The reaction chamber is separated from a feeding solution by one or several dialysis membranes, allowing constant exchange of substrates and by-products. See, e.g., Endo et al., J. Biotechnol. 25:221, 1992; and Spirin et al., Science 24:1162, 1988.

The Feeding Solution and other compositions of the invention can be applied in full or in part to any type of IVPS system, including any of the four above-listed IVPS reactions. Preferably, a feeding solution comprises 1) a buffer, 2) amino acids, and 3) at least one energy source or energy generating enzyme.

A representative Feeding Solution of the invention contains several, but not necessarily all, of the following:
(a) a buffer (10-500 mM, preferably 10-100 mM);
(b) one or more salts, including Ammonium acetate at 10-500 mM, preferably 60-120 mM.
(c) one or more reducing agents;
(d) at least 4 amino acids; and
(e) one or more energy sources and/or cofactor;

Each of these components is described in more detail below:

(a) Buffers: A buffer is included in the Feeding Solution in order to maintain the pH of the reaction. The same buffer is typically, but need not be, used in both the initial reaction mix and the Feeding Solution. The pH of the buffer of a Feeding Solution may vary from that of the initial reaction mix. Nonlimiting examples of buffers include Tris, Bis-tris and HEPES.

In some embodiments of the invention, HEPES buffer at from 10-100 mM final concentration is included in the feeding solution to maintain the pH of the reaction. The pH of the feeding solution buffer can be from about 7 to about 9, but preferably is between about 7.5 and about 8.5. In an exemplary embodiment, the pH of the buffer is about 8.0.

(b) Reducing Agents can include without limitation tris(2-carboxyethyl)phosphine (TCEP), glutathione, dithiothreitol (DTT) and β-mercaptoethanol. See Getz et al., Analytical Biochemistry 273, 73-80 (1999).

(c) Salts: A salt is a neutral compound formed by the union of an acid (or cations thereof) and a base or a metal. Salts are named according to their constituent ions. The cationic components, often metal ions (e.g., Ca⁺⁺, Mg⁺⁺, Mn⁺⁺) or ammonium (NH4⁺), are given first, followed by the anionic (negatively charged) components. The cation can be monovalent (+1), divalent (+2), trivalent (+3), etc. Monovalent cations include without limitation H⁺ and K⁺. Divalent cations include without limitation Ca⁺⁺, Zn⁺⁺, Hg⁺⁺, Mn⁺⁺, Mg⁺⁺, Ba⁺⁺ and Sr⁺⁺. In many in vivo and in vitro biochemical reactions, divalent cations are co-factors. More particularly, Ca⁺⁺, Mn⁺⁺ and Mg⁺⁺ are frequent co-factors of enzymatic reactions and are thus preferred in some biochemical systems.

An anion can be monovalent (−1), divalent (−2), trivalent (−3), etc. Anions are typically named according to the their conjugate acid, for example, acetates, carbonates, chlorides, cyanides, nitrates, nitrites, phosphates, sulfates, and citrates.

Any of the above non-limiting examples of anions can be part of the salts used in compositions of the invention. Amino acid salts can be used as well, e.g. potassium glutamate.

Preferred salts include without limitation magnesium salts, such as at 5-50 mM, preferably 10-15 mM; potassium glutamate, 180-250 mM, preferably 230 mM; CaCl₂, 1 to 750 mM, preferably 5, 10, 20, 30, 50 or 100 mM; and ammonium acetate at from 10-500 mM, preferably 60-120 mM, and more preferably about 70-90 mM. In some aspects of the invention, potassium acetate can substitute for potassium glutamate.

The salts included in the feeding solution can be the same as those provided in the initial reaction buffer, or additional salts (for example, calcium chloride) can be added. Salts can also be provided at different concentrations in the feeding solution, to increase or decrease the overall concentration in the reaction once the feeding solution has been added. The addition of calcium to a feeding buffer, for example, generates an increase in yields of about 10% by raising the calcium concentration from 0.1 to 10 mM, preferably from 0.5 to 5 mM, and more preferably from about 1 to 2.5 mM in the IVPS reaction.

(d) Amino acids are present in a feeding solution at 0.05 to 5.0 mM, preferably 0.25-2.5 mM, more preferably yet from 0.5 to 2 mM, and even more preferably from 1.0 to 1.5 mM. All 20 naturally-occurring amino acids or a subset may be provided in the feeding buffer. In some preferred embodiments, all 20 are provided. One or more amino acids may be provided at a higher or lower concentration that the others. For example, in some cases protein synthesis may be more efficient when one or more amino acids is present at a higher concentration than the others. In other cases, particularly, a protein is to be labeled using a modified amino acid. In this case the cognate naturally-occurring amino acid can be provided at a lesser concentration in the feeding solution, or omitted from the feeding solution.

In some embodiments of the invention, IVPS reactions are use to efficiently and specifically add detectably labeled and/or unnatural amino acids into the protein of interest, and one or more amino acids is provided in labeled or modified form, or an unnatural or modified amino acid is substituted for a “standard” amino acid. A detectably labeled amino acid can result from its conjugation with a fluorescent moiety, such as fluorescein 5-isothiocyanate (FITC); conjugation with biotin or streptavidin; and heavy isotope or radiolabeled amino acids including without limitation, ¹⁵N-labeled amino acids, ³⁵S-labeled amino acids, ¹⁴C-labeled amino acids; and ²H-labeled amino acids.

(e) Energy sources such as, but not limited to, glycolytic intermediates, or other phosphate-carrying molecules, such as, but not limited to, acetyl phosphate, creatine phosphate, or phospho-arginine. An energy source can be a substrate molecule (such as a glycolytic intermediate) or an enzyme, such as, for example, hexokinase, pyruvate kinase, arginine kinase, or pyruvate oxidase (see, for example, U.S. Pat. Nos.6,168,931, and 6,337,191, both herein incorporated by reference for all disclosure of energy sources and energy-generating enzymes and systems).

In the present invention, glycolytic intermediates are preferred energy sources for inclusion in a feeding solution (see, for example, U.S. Pat. No. 6,337,191, herein incorporated by reference for all disclosure of glycolytic intermediates as energy sources in IVPS systems). Glycolytic intermediates such as but not limited to 3-Phosphoglycerate, phosphoenolpyruvate, Fructose-6-Phosphate, or Glucose-6-Phosphate, or other glycolytic intermediates can be added at 1-200 mM, preferably 10-100 mM.

In the present invention, a preferred energy source for inclusion in a feeding solution is an energy source molecule that is not provided in the initial reaction buffer. For example, a preferred initial reaction IVPS buffer includes acetyl phosphate and phosphoenolpyruvate. The inventors have found that adding an additional energy source molecule that is different from those provided at t=0, provides better translational yields than adding more of the same energy source molecules (e.g., acetyl phosphate and phosphoenolpyruvate). Preferably, the additional energy source molecules provided in the feeding solution is a glycoytic intermediate that is not provided in the base reaction buffer. Preferred glycolytic intermediates include without limitation Phosphoenol Pyruvate, Acetyl Phosphate, Glucose 6 Phosphate, Fructose 6 Phosphate, and 3 Phosphoglycerate individually, or in combination with each other. The optimal total concentration of a glycolytic intermediate in the IVPS reaction is 20 mM-60 mM, preferably not to exceed 80 mM.

Preferably, the initial or “base” reaction IVPS buffer includes at least two energy sources, and the feeding solution includes at least one energy source different from the energy sources of the base reaction buffer, such that the reaction, after the addition of feeding solution, includes at least three different added energy sources. Preferably, the energy source added in the feeding solution is a glycolytic intermediate that provides at least one of: enhanced protein yield, enhanced soluble protein yield, or enhanced active protein yield, when added to an IVPS system. In preferred embodiments, none of the one or more energy sources added in the feeding solution is an enzyme. This avoids issues of enzyme stability in the feeding buffer, allowing for a single feeding reagent to be added to the reaction, and also avoids the expense of enzymes.

In some preferred embodiments, none of the energy sources added in the base reaction IVPS buffer or the feeding buffer are enzymes. The convenience of avoiding the use of enzymes in the feeding buffer applies also to the initial reaction. However, in a preferred embodiment one or more energy source generating enzymes can be added to the S30 extract prior to addition of the IVPS reagents, for example prior to or during pre-incubation of the extract which can be performed prior to NT reactions, and preferably before aliquoting and storage of the extract. For example, pyruvate kinase can be added to an S30 extract before preincubation of the extract to provide an energy source for “running off” endogenous messages. Thus, an in vitro translation system used in the methods of the present invention can have at least four different added energy sources. For example, an in vitro translation system can have at least four different added energy sources, in which at least one of which is a glycolytic intermediate. Further, in preferred embodiments, an in vitro translation system can have at least four different added energy sources, in which at least one of which is an enzyme and at least one other of which is a glycolytic intermediate. In preferred embodiments, an in vitro translation system can have at least four different added energy sources, in which at least one of which is an enzyme and at least one other of which is a glycolytic intermediate that is added after the initiation of the IVPS reaction to enhance the performance of the IVPS system. In the methods of the present invention, the addition of at least one glycolytic intermediate to the in vitro translation reaction in a feeding solution added at least ten minutes after the initiation of the reaction enhances the yield, soluble yield, or active yield of a protein synthesized in the system. In a very preferred embodiment, an in vitro translation system can have at least four different added energy sources, in which at least one of which is an enzyme and at least one other of which is a glycolytic intermediate added after the initiation of the IVPS reaction to enhance the performance of the IVPS system, wherein the glycolytic intermediate added after the initiation of the reaction is different from any of the other energy sources added to the system. In the methods of the present invention, the addition of at least one glycolytic intermediate to the in vitro translation reaction in a feeding solution added at least ten minutes after the initiation of the reaction enhances one or more of the yield, soluble yield, or active yield of a protein synthesized in the system.

The addition of a glycolytic intermediate in a feeding solution without the co-factor NAD or NADH can enhance the activity of synthesized proteins, and together with NAD or NADH, such as, but not limited to: NAD or NADH at 0.1-25 mM, preferably 0.1-1 mM, can stimulate both better expression and activity or synthesized proteins. These effects can be observed through a range of concentrations of each component: Amino acids 1 mM-5 mM; glycolytic intermediates 5 mM-100 mM, and NADH 0.1 mM-1 mM.

In one aspect, the present invention provides potent Feeding Solutions having many desirable features (e.g., providing greater yield of protein, proteins with increased solubility, shorter protein synthesis reaction times for equivalent or greater protein yield, and the like).

In another aspect, the present invention also includes methods of performing in vitro protein synthesis, in which a feeding solution is added to an ongoing protein synthesis reaction that includes a cell extract and a nucleic acid template, and reagents sufficient for the production of protein.

The initial synthesis mixture includes sufficient reagents to allow protein synthesis to occur for at least 30 mM, and preferably at least 60 min, in the absence of feeding buffer. A feeding solution is added to enhance ongoing protein synthesis. The invention can be applied to any type of IVPS system or technology (e.g., batch reaction, feeding/dilution, bilayer overlay, continuous exchange, etc.). In a Feeding/Dilution IVPS system, an IVPS reaction is supplemented with a Feeding Solution some time after the reaction is started (t=0).

In one embodiment, the method includes adding to a cell extract: amino acids, at least one energy source, and a nucleic acid template, to make an initial synthesis mixture; incubating the initial synthesis mixture for a period of time; adding to the initial synthesis mixture a feeding solution that comprises a buffer, amino acids, and at least one additional energy source, wherein one or more additional energy sources added in the feeding solution are different from the one or more energy source of the initial synthesis mixture, to make an extended synthesis mixture; and incubating the extended synthesis mixture for an additional period of time to synthesize at least one protein.

In preferred embodiments the feeding solution used in these methods includes a buffer; one or more salts; at least four, preferably at least fifteen, and more preferably twenty, amino acids, one or more of which can be non-naturally occurring (for example, labeled or modified); and a glycolytic intermediate energy source. Preferably, the feeding solution also includes a cofactor such as, but not limited to, NAD or NADH.

The methods of using feeding solutions disclosed herein serve a two-fold purpose in such in vitro reactions. First, the IVPS reaction is supplemented with new components; both components that have been depleted or degraded during the reaction and/or new components not present in the original reaction. Second, any inhibitory byproducts are diluted by the addition of buffer, thus prolonging the synthesis reaction. Extensive dilution of transcription and/or translation factors, however, may cause a decrease or loss of the activity of the IVPS system. The compositions of the invention are prepared in concentrated form in order to avoid excessive dilution of transcription and/or translation factors may cause a decrease or loss of the activity of the IVPS system.

Any volume of feeding buffer can be added to the initial synthesis mixture, for example, from one-tenth the initial synthesis mixture volume to ten times the initial synthesis mixture volume. Preferably, from one-fourth the initial synthesis mixture volume to two times the initial synthesis mixture volume is added in a feed. Even more preferably, one or more feed of from one-half volume to one-volume of the original IVPS volume are added to an IVPS reaction. It is also contemplated, however, that the presence of three or more energy sources within a single IVPS reaction, regardless of whether they are added at the outset of the reaction or whether one or more energy sources is provided in a feeding solution, is an aspect of the present invention. The invention thus encompasses IVPS systems comprising three or more energy sources, at least one of which is a glycolytic intermediate. In some preferred embodiments an IVPS system comprises three or more energy sources, at least one of which is a glycolytic intermediate, and at least one of which is an enzyme. The invention also includes IVPS systems comprising four or more energy sources. In some preferred embodiments, an IVPS reaction comprises four or more energy sources, at least one of which is a glycolytic intermediate. In some preferred embodiments, an IVPS system comprises four or more energy sources, at least one of which is a glycolytic intermediate, and at least one or which is an enzyme.

The invention provides methods that can synthesize milligram quantities of a protein of interest (P01) by adding a feeding solution to an IVPS reaction. Preferably the protein is synthesized at a concentration from at least 1 to about 1 mg/ml or more preferably from about 100 mg/ml to about 800 mg/mL, in from about 1 hour to about 10 hours of IVPS reaction time, preferably in from about 2 hours to about 8 hours of IVPS reaction time, and more preferably yet, in form about 3 hours to about 7 hours of total IVPS reaction time. For example, reactions of from 0.5 to 5 ml (final volume after one or more feeds) reactions can be used to synthesize milligram quantities of proteins in four to six hours. Exemplary IVPS reactions that use feeding solutions are used to synthesize at least 0.8 milligram, and more preferably at least 1 milligram, of protein in a final reaction volume of from 1 to 2 m is in 4 to 6 hours.

It should be understood that compositions denoted as “Feeding Solutions” herein could also be used in IVPS systems or steps that do not involve feeding and/or dilution. For example, a composition described herein as a Feeding Solution of the invention might also be introduced at t=0 in a batch reaction, or continuously or intermittently in a continuous exchange system, etc.

The methods of the present invention include adding feeding solution once, twice, or more times to an IVPS reaction. Preferably, for convenience, one or two feeds are performed. For example, feeding solution can be added to an IVPS at two time points, where each feed has a volume of half that of the initial synthesis mixture. Alternatively, a single feed can be provided of a volume equal to that of the initial synthesis mixture. These examples are not intended to be limiting.

The Feeding Solution may be added at any time during the IVPS reaction; however preferably at least one feed occurs within the first hour after the reaction has been initiated. As described in the Examples that follow, reactions in which the first feed occurs not later than one hour after the IVPS has been initiated result in better protein yields than those with initial feeds that occur later. Nevertheless, providing feed buffer at the initiation of the reaction is not optimal, possibly due to dilution of essential components. Thus, the first addition of a feeding solution occurs at least five minutes after the IVPS is initiated, preferably at least 10 minutes after the IVPS is initiated. A feeding solution can be added, for example, 15 minutes after the IVPS is initiated or later. After one hour, the addition of a first feed is less effective. Preferably therefore, where one or more feeds are performed, the first feed occurs from about 15 to about 60 minutes after the IVPS is initiated. A second feed, if used, can be added at any time, preferably at least 30 min, and more preferably at least 60 min, after a first feed.

### Addition of Detergents to IVPS Extracts and Reactions

In some aspects of the invention, one or more lipids, surfactants, or detergents are included in IVPS cell extracts or IVPS reaction mixtures. One or more lipids, surfactants, or detergents can enhance the solubility or activity of some proteins, such as, but not limited to, membrane proteins. The use of combinations of one or more lipids and one or more surfactants, one or more lipids and one or more detergents, one or more surfactants and one or more detergents, and combinations of one or more lipids, one or more surfactants, and one or more detergents in an IVPS system is also contemplated.

Preferred lipids are phospholipids, which can be glycerol or sphingolipid based, and can contain, for example, two saturated fatty acids of from 6 to 20 carbon atoms and a commonly used head group such as, but not limited to, phosphatidyl choline, phosphatidyl ethanolamine and phosphatidyl serine. The head group can be uncharged, positively charged, negatively charged or zwitterionic. The phospholipids can be natural (those which occur in nature) or synthetic (those which do not occur in nature), or mixtures of natural and synthetic. Examples of phospholipids include, without limitation, PC, phosphatidyl choline; PE, phosphatidyl ethanolamine, PI, phosphatidyl inositol; DPPC, dipalmitoyl-phosphatidylcholine; DMPC, dimyristoyl phosphatidyl choline; POPC, 1-palmitoyl-2-oleoyl-phosphatidyl choline; DHPC, dihexanoyl phosphatidyl choline, dipalmitoyl phosphatidyl ethanolamine, dipalmitoyl phosphatidyl inositol; dimyristoyl phosphatidyl ethanolamine; dimyristoyl phosphatidyl inositol; dihexanoyl phosphatidyl ethanolamine; dihexanoyl phosphatidyl inositol; 1-palmitoyl-2-oleoyl-phosphatidyl ethanolamine; or 1-palmitoyl-2-oleoyl-phosphatidyl inositol; among others.

Nondetergent surfactants, such as but not limited to the non-detergent sulfobetaines (NDSBs) can be included in IVPS reactions. The NDSBs are zwitterionic compounds that have a sulfobetaine hydrophilic group and a short hydrophobic group. They cannot aggregate to form micelles, and NDSBs are thus not considered detergents.

Detergents, including ionic, non-ionic, and zwitterionic detergents can also be included in IVPS reactions. Non-ionic and Zwitterionic detergents are preferred in most aspects of the invention. In some embodiments, a detergent provided in an IVPS reaction is preferably an a non-ionic or zwitterionic detergent having a critical micelle concentration of 15-300 mM and, more preferably, 20-50 mM.

Anionic Detergents Include without Limitation

Glycochenodeoxycholic acid sodium salt; Glycocholic acid hydrate, synthetic; Glycocholic acid sodium salt hydrate; Glycodeoxycholic acid monohydrate; Glycodeoxycholic acid sodium salt; Glycoli thocholic acid 3-sulfate disodium salt; and Glycolithocholic acid ethyl ester;

Sodium 1-butanesulfonate; Sodium 1-decanesulfonate; Sodium 1-dodecanesulfonate; Sodium 1-heptanesulfonate; Sodium 1-nonanesulfonate; Sodium 1-propanesulfonate monohydrate; and Sodium 2-bromoethanesulfonate;

Sodium cholate hydrate; Sodium choleate; Sodium deoxycholate; Sodium dodecyl sulfate; Sodium hexanesulfonate; Sodium octyl sulfate; Sodium pentanesulfonate; and Sodium taurocholate;

Taurochenodeoxycholic acid sodium salt; Taurodeoxycholic acid sodium salt monohydrate; Taurohyodeoxycholic acid sodium salt hydrate; Taurolithocholic acid 3-sulfate disodium salt; and Tauroursodeoxycholic acid sodium salt;

As well as Chenodeoxycholic acid; Cholic acid, ox or sheep bile; Dehydrocholic acid; Deoxycholic acid methyl ester; Digitonin; Digitoxigenin; N,N-Dimethyldodecylamine N-oxide; Docusate sodium salt; N-Lauroylsarcosine sodium salt; Lithium dodecyl sulfate; Niaproof 4, Type 4; 1-Octanesulfonic acid sodium salt; Trizma® dodecyl sulfate; and Ursodeoxycholic acid.

Cationic detergents include without limitation:

Alkyltrimethylammonium bromide; Benzalkonium chloride; Benzyldimethylhexadecylammonium chloride; Benzyldimethyltetradecylammonium chloride; Benzyldodecyldimethylammonium bromide; and Benzyltrimethylammonium tetrachloroiodate; Dimethyldioctadecylammonium bromide; Dodecylethyldimethylammonium bromide; Dodecyltrimethylammonium bromide; Ethylhexadecyldimethylammonium bromide; Hexadecyltrimethylammonium bromide; Thonzonium bromide; and Trimethyl(tetradecyl)ammonium bromide.

Non-ionic detergents include without limitation:

Brij® detergents, including without limitation Brij® 35; Brij® 56; Brij® 58P; Brij® 72; Brij® 76; Brij® 92V; Brij® 97; and Brij® 58P;

Span® detergents, including without limitation Span® 20; Span® 40; Span® 60; Span® 65; Span® 80; and Span® 85;

Triton detergents, including without limitation Triton CF-21; Triton CF-32; Triton DF-12; Triton DF-16; Triton GR-5M; Triton QS-15; Triton QS-44; Triton X-100; Triton X-102; Triton X-15; Triton X-151; Triton X-200; Triton X-207; Triton® X-100; Triton® X-114; Triton® X-165; Triton® X-305; Triton® X-405; Triton® X-45; and Triton® X-705;

Tergitol detergents, including without limitation Tergitol, Type 15-S-12; Tergitol, Type 15-S-30; Tergitol, Type 15-S-5; Tergitol, Type 15-S-7; Tergitol, Type 15-S-9; Tergitol, Type NP-10; Tergitol, Type NP-4; Tergitol, Type NP-40; Tergitol, Type NP-7; Tergitol, Type NP-9; Tergitol, Type TMN-10; and Tergitol Type TMN-6;

TWEEN® detergents, including without limitation TWEEN® 20; TWEEN® 21; TWEEN® 40; TWEEN® 60; TWEEN® 61; TWEEN® 65; TWEEN® 80; TWEEN® 80; TWEEN® 81; and TWEEN® 85.

Mega detergents, including without limitation Mega-8 and Mega-10;

N-Decanoyl-N-methylglucamine; n-Decyl a-D-glucopyranoside; Decyl beta-D-maltopyranoside; n-Dodecanoyl-N-methylglucamide; n-Dodecyl a-D-maltoside; n-Dodecyl-beta-D-maltoside; and n-Hexadecyl-beta-D-maltoside;

Heptaethylene glycol monodecyl ether; Heptaethylene glycol monododecyl ether; and Heptaethylene glycol monotetradecyl ether;

Hexaethylene glycol monododecyl ether; Hexaethylene glycol monohexadecyl ether; Hexaethylene glycol monooctadecyl ether; and Hexaethylene glycol monotetradecyl ether;

Octaethylene glycol monodecyl ether; Octaethylene glycol monododecyl ether; Octaethylene glycol monohexadecyl ether; Octaethylene glycol monooctadecyl ether; and Octaethylene glycol monotetradecyl ether; Octyl-b-D-glucopyranoside;

Pentaethylene glycol monodecyl ether; Pentaethylene glycol monododecyl ether; Pentaethylene glycol monohexadecyl ether; Pentaethylene glycol monohexyl ether; Pentaethylene glycol monooctadecyl ether; and Pentaethylene glycol monooctyl ether;

Polyethylene glycol diglycidyl ether; and Polyethylene glycol ether W-1;

Polyoxyethylene 10 tridecyl ether; Polyoxyethylene 100 stearate; Polyoxyethylene 20 isohexadecyl ether; and Polyoxyethylene 20 oleyl ether;

Polyoxyethylene 40 stearate; Polyoxyethylene 50 stearate; Polyoxyethylene 8 stearate; Polyoxyethylene bis(imidazolyl carbonyl); and Polyoxyethylene 25;

Tetraethylene glycol monodecyl ether; Tetraethylene glycol monododecyl ether; and Tetraethylene glycol monotetradecyl ether;

Triethylene glycol monodecyl ether; Triethylene glycol monododecyl ether; Triethylene glycol monohexadecyl ether; Triethylene glycol monooctyl ether; and Triethylene glycol monotetradecyl ether;

Phosphine oxides, such as APO-9, APO-10; APO-12;

As well as Bis(polyethylene glycol bis[imidazoyl carbonyl]); Cremophor® EL; Decaethylene glycol monododecyl ether; Tyloxapol; and n-Undecyl-beta-D-glucopyranoside; Igepal CA-630; Methyl-6-O-(N-heptylcarbamoyl)-a-D-glucopyranoside; Nonaethylene glycol monododecyl ether; N-Nonanoyl-N-methylglucamine; NP-40; propylene glycol stearate; Saponins, e.g., Saponin from Quillaj a bark; and Tetradecyl-b-D-maltoside.

Zwitterionic detergents include without limitation:

Zwittergent® detergents, including without limitation Zwittergent® 3-12 (3-Dodecyl-dimethylammonio-propane-1-sulfonate); Zwittergent® 3-08; Zwittergent® 3-10; Zwittergent® 3-14; and Zwittergent® 3-16; 3-(Decyldimethylammonio)propanesulfonate inner salt; 3-(Dodecyldimethylammonio)propanesulfonate inner salt; 3-(N,N-Dimethylmyristylammonio)propanesulfonate; 3-(N,N-Dimethyloctadecylammonio)propanesulfonate; and 3-(N,N-Dimethylpalmitylammonio)propanesulfonate;

as well as BigCHAP; CHAPS; CHAPSO; dimethyl-dodecylamine; DDMAU; Lauryldimethylamine oxide (LADAO, LDAO); and N-Dodecyl-N,N-dimethylglycine;

In some embodiments of the invention, one or more phospholipids, surfactants, or detergents is present in an IVPS reaction mixture by being added directly to the reaction mix. One or more detergents, surfactants, or phospholipids, or combinations thereof, can also be used in the feeding solutions of the invention.

In some aspects of the present invention, nanoscale phospholipid bilayer discs can be included in the IVPS reaction mixture. Such phospholipid-protein particles or “nanodiscs” that include phospholipids in a bilayer structure engirdled by a scaffold protein such as Apolipoprotein A1 (Apo-A1) or derivatives thereof, have been described by Bayburt et al. (J. Struct. Biol. 123: 37-44 (1998)) and Bayburt and Sligar (PNAS 99:6725-6730 (2002); Protein Science 12:2476-2481 (2004)) and are disclosed in U.S. Patent Application Publication No. 2005/0182243, herein incorporated by reference for all disclosure related to nanoscopic phospholipid bilayer discs and their components, such as phospholipids and scaffold proteins. The inclusion of nanoscopic phospholipid bilayer discs can improve the yield of soluble protein, particularly when membrane proteins are synthesized in IVPS reactions. For example, nanoscopic phospholipid bilayer discs can be included in an IVPS reaction, such as those described herein, at a concentration of from 0.1 to 100 mm, preferably from 0.2 to 50 m, and more preferably yet from 0.5 mm to 40 mm. For example, nanodiscs can be present in an IVPS reaction at from about 1 mm to about 20 mm.

Including nanoscopic phospholipid bilayer discs in an IVPS reaction can increase the solubility of in vitro translated membrane proteins. Membrane proteins (including integral, embedded, and peripheral membrane proteins), can be in vitro translated in the presence of nanodiscs, such that the membrane proteins are inserted into the nanoscopic phospholipid bilayer discs. In some aspects of the invention, the nanodisc-inserted membrane proteins can be isolated using affinity tags provided on the scaffold protein of the nanodisc.

In other preferred embodiments of the invention, one or more phospholipids, surfactants, or detergents are present in an IVPS reaction mixture by having been added to cells or a cell lysate during preparation of a cell extract for IVPS. A phospholipid, surfactant, or detergent is preferably added to cells prior to lysis or to a cell lysate prior to removal of cell debris from the lysate. As demonstrated in the Examples, addition of a detergent to cells or a cell lysate prior to the removal of cell debris from the cell lysate can result in a cell extract that produces greater amounts of soluble protein than extracts made without detergent present. Thus, in the methods of the present invention, the membranes and cellular debris that are separated from the cell lysate during extract preparation supernatant (for example, by centrifugation, filtration, chromatography, etc.) are exposed to one or more detergents, surfactants, or added lipids prior to their removal from the cell lysate.

Although the invention is not limited to a particular mechanism, it is contemplated that when an extract is prepared using methods of the present invention, certain peripheral membrane proteins, aggregated proteins, or other biomolecules that are removed by centrifugation during standard IVPS extract preparation are solubilized by detergent treatment such that they separate into the supernatant during cell lysate centrifugation. These solubilized components therefore become part of the cell lysate supernatant that is separated from cellular debris for use as a cell extract in IVPS. Such solubilized proteins or biomolecules can improve the yield or promote the solubilzation or enhance the solubility or activity of in vitro synthesized proteins.

Nondetergent surfactants and/or phospholipids can also promote the release of biomolecules or factors that promote protein synthesis, folding, or solubilization. The present invention also includes IVPS systems having extracts that include one or more surfactants, one or more detergents, or one or more lipids, such as but not limited to one or more phospholipids, in which the one or more surfactants, detergents, or phospholipids has been added to the cells used to make the extract prior to lysing the cells, or has been added to the cell lysate used to make the cell extract prior to removal of cell debris from the cell lysate.

The present invention includes a cell extract for use in an IVPS system that includes a detergent, surfactant, or lipid, in which the cell extract is made by lysing cells to obtain a cell lysate and removing cell debris from the cell lysate, in which one or more detergents, surfactants, or lipids is added to the cells prior to lysis or to the cell lysate prior to removing cell debris from the lysate. As used herein “cell debris” can include components of a lysate such as but not limited to: fragments of cell wall, fragments of cell membrane, fragments of genomic DNA, or large aggregates of biomolecules that can be removed from a lysate based on properties such as size or density using methods that do not substantially remove free ribosomes from the lysate. Preferably cell debris is removed from a lysate using methods such as centrifugation or filtration, most preferably centrifugation.

Filtration, selective precipitation, affinity capture, or chromatography can optionally be used instead of or in addition to centrifugation as a method for separating cell debris or undesirable materials from a cell lysate to be used as a cell extract in IVPS. Methods of making a cell extract for IVPS are known in the art for various eukaryotic and prokaryotic systems. The present invention can be applied to any of these methods or methods developed in the art in that use cell extracts for IVPS, in which cells are lysed and cell debris and/or other undesirable components are removed from the lysate to produce an extract for IVPS. Removal of cell debris and/or undesirable components can be by methods such as centrifugation, filtration, chromatography, affinity capture, etc.

In some preferred aspects of the invention, a detergent or surfactant is added to the buffer in which cells are lysed or to a lysate prior to removal of cell debris from the lysate. In some preferred aspects of the invention, a detergent is added to the buffer in which cells are lysed or to a lysate prior to removal of cell debris from the lysate. Preferably, the detergent is a nonionic detergent or a zwitterionic detergent.

A nonionic detergent used to make an IVPS extract of the present invention can be, as nonlimiting examples, a glycopyranoside (or glucopyranoside), a detergent of the Brij series, a detergent of the Triton series, a nonidet detergent, or a Tween detergent. Some preferred nonionic detergents are glycopyranosides (or glucopyranosides), such as, for example, dodecyl maltoside, octylglucopyranoside, or octylthioglucopyranoside; Brij detergents, such as, for example, Brij® 35 m or Triton detergents, such as, for example, Triton-X 100. A zwitterionic detergent used to make an IVPS extract of the present invention can be, as nonlimiting examples, a sulfobetaine detergent, a detergent of the Zwittergent® series, a detergent of the EMPIGEN® series, CHAPS, or CHAPSO, for example, Zwittergent® 3-14 or CHAPS.

Detergents can be used in combination with other detergents, with one or more surfactants, with one or more lipids (such as, but not limited to, phospholipids), or any combination of one or more of additional detergents, one or more surfactants, or one or more lipids.

TABLE 2
EXEMPLARY DETERGENTS
NON-IONIC DETERGENTS

| | MW | CMC | AGGREGATION | MW |
|---|---|---|---|---|
| DETERGENT NAME | (monomer) | (mM)* | NUMBER | (MICELLE) |
| APO-12 | 246.4 | 0.568 | 2,232 | 549,965 |
| TRITON X-100 (tert-C8-Ø- | 650 (avg) | 0.3 | 140 | 90,000 |
| E9.6) | | | | |
| TWEEN 80 (C18:1-sorbitan- | 1310 (avg) | 0.012 | 58 | 75,980 |
| E20) | | | | |
| Digitonin | 1229.3 | | 60 | 70,000 |
| Nonidet P-40 (NP-40) | 603.0 | 0.05-0.3 | 100-155 | 60,300-93,465 |
| n-Dodecyl-β-D- | 348.5 | 0.13 | | 70,000 |
| glucopyranoside | | | | |
| n-Dodecyl-beta-D-maltoside | 348.5 | 0.15 | 98 | 70,000 |
| APO-10 | 218.3 | 4.6 | 131 | 28,597 |
| n-Octyl-beta-D- | 292.4 | 25 | 27 | 7,895 |
| glucopyranoside | | | | |

ZWITTERIONIC DETERGENTS

| | MW | CMC | AGGREGATION | MW |
|---|---|---|---|---|
| DETERGENT NAME | (MONOMER) | (mM)* | # | (MICELLE) |
| ZWITTERGENT 3-16 | 391.6 | 0.01-0.06 | 155 | 60,700 |
| ZWITTERGENT 3-14 | 363.6 | 0.1-0.4 | 83 | 30,200 |
| ZWITTERGENT 3-12 (3- | 335.6 | 2-4 | 55 | 18,500 |
| Dodecyl-dimethylammonio- | | | | |
| propane-1-sulfonate) | | | | |
| Lauryldimethylamine oxide | 229.4 | 1-3 | 76 | 17,000 |
| (LADAO, LDAO, Empigen OB) | | | | |
| ZWITTERGENT 3-10 | 307.6 | 25-40 | 41 | 12,600 |
| CHAPSO | 630.9 | 8 | 11 | 9,960 |
| BigCHAP | 878.1 | 3.4 | 10 | 8,800 |
| CHAPS | 614.9 | 6-10 | 10 | 6,150 |

*CMC at 50 mM Na+ unless otherwise stated.

In one aspect, the invention includes a method of making an extract for protein synthesis comprising: resuspending cells in a buffer; lysing the cells to obtain a lysate; adding one or more detergents, surfactants, or phospholipids, to the lysate; and removing cell debris from the lysate to provide an extract for protein synthesis. In preferred methods, removing cell debris comprises centrifuging the lysate and removing at least a portion of the supernatant that includes ribosomes to provide a cell extract for protein synthesis. The cells can be prokaryotic or eukaryotic cells.

In preferred embodiments, one or more detergents or surfactants is added to a cell lysate prior to the separation of cell debris from the cell lysate used as a cell extract for IVPS. For example, one or more detergents can be added to a cell lysate prior to the separation of cell debris from the cell lysate used as a cell extract for IVPS. Preferably, when a detergent is used in preparing an extract, the detergent is used at a concentration such that after adding a detergent to a cell lysate, the cell lysate has a detergent concentration at or above the detergent's CMS. In some preferred embodiments, when a detergent is used in preparing an extract, the detergent is used at a concentration such that after adding a detergent to a cell lysate, the cell lysate has a detergent concentration less than twice the detergent's CMC.

The present invention includes an in vitro protein synthesis system that includes a cell extract that includes at least one detergent, surfactant, or lipid, in which the cell extract is made by lysing cells to obtain a cell lysate and removing cell debris from the cell lysate, in which one or more detergents, surfactants, or lipids is added to the cell lysate prior to removing cell debris from the lysate. In some preferred embodiments, the present invention includes an in vitro protein synthesis system that includes a cell extract that includes at least one detergent, in which the extract is made by adding one or more detergents to a cell lysate prior to removing cell debris from the lysate.

In some aspects of the invention, cells used to make an TVPS extract are exposed to an added detergent, surfactant, or lipid prior to lysis of the cells. The added detergent, surfactant, or lipid is not used at sufficient concentration or strength to cause lysis of the cells. For example, one or more detergents, surfactants, or lipids can be added to a cell suspension, after which the cells are lysed. In preferred embodiments, a detergent, surfactant, or phospholipid is added to a buffer in which cells are lysed to make an IVPS extract. The invention includes a method of making an extract for protein synthesis comprising resuspending cells in a buffer that includes at least one detergent, surfactant, or phospholipids; lysing the cells to obtain a lysate; and separating cell debris from the lysate to make a cell extract for use in IVPS. In preferred methods, separating cell debris comprises centrifuging the lysate and removing at least a portion of the supernatant to provide a cell extract for protein synthesis. The cells can be prokaryotic or eukaryotic cells.

In some preferred embodiments, one or more detergents or surfactants is added to intact cells used for preparing an extract for IVPS. One or more detergents, for example, can be added to intact cells prior to their lysis. For example, a cell pellet can be resuspended in a buffer, and one or more detergents can be added to the resuspension. Preferably, a detergent is added in an amount such that the cell suspension has a final detergent concentration at or above the detergent's CMC. Alternatively, a cell pellet can be resuspended in a buffer that includes one or more detergents, where the concentration of a detergent in the buffer is preferably at or above the detergent's CMC. In some preferred embodiments, when a detergent is provided in a cell suspension prior to lysing the cells to make an IVPS extract, the concentration of a detergent present in the cell suspension is less than twice the detergent's CMC.

The present invention includes an in vitro protein synthesis system that includes a cell extract that includes a detergent, surfactant, or lipid, in which the cell extract is made by lysing cells to obtain a cell lysate and removing cell debris from the cell lysate, in which one or more detergents or surfactants is added to the cells prior to lysis. In some preferred embodiments, the present invention includes an in vitro protein synthesis system that includes a cell extract that includes at least one detergent, in which the cell extract is made by lysing cells to obtain a cell lysate and removing cell debris from the cell lysate, in which the cells are exposed to the one or more detergents prior to lysis.

The present invention includes a method of synthesizing a protein in vitro, in which the cell lysate used to make the extract used in the IVPS reaction has been treated with at least one lipid, at least one surfactant, or at least one detergent prior to removing cell debris from the cell lysate. The method includes: adding amino acids, at least one energy source, and a nucleic acid template to a cell extract to make an in vitro protein synthesis mixture; where the cell extract is made from cells or a cell lysate that has been treated with at least one lipid, surfactant or detergent prior to making the extract; and incubating the vitro protein synthesis mixture to synthesize the protein. In practicing the method, protocols for IVPS as they are known in the art or improved or optimized in the future can be used. The cell extract can be made from prokaryotic or eukaryotic cells. The method can be applied to batch IVPS, continuous exchange IVPS, bilayer overlay IVPS, or feeding/dilution IVPS. The IVPS system can use an RNA or DNA template.

In some embodiments, the method uses a cell extract that is made by treating a cell lysate with one or more detergents, surfactants, or lipids prior to removing cell debris from the cell lysate. In some preferred embodiments, the method uses a cell extract that is made by treating a cell lysate with one or more detergents prior to removing cell debris from the cell lysate. In some preferred embodiments, the cell lysate is treated with one or more zwitterionic detergents or one or more nonionic detergents, such as those disclosed herein.

In some embodiments, the method uses a cell extract that is made by treating cells with one or more detergents, surfactants, or lipids prior to lysing the cells. In some preferred embodiments, the method uses a cell extract that is made by treating cells with one or more detergents prior to lysing the cells. In some preferred embodiments, the cells are treated with one or more zwitterionic detergents or one or more nonionic detergents, such as those disclosed herein.

The methods can further include adding a feeding solution that includes a buffer, amino acids, and at least one energy source other than an energy source present in the initial translation reaction to the in vitro translation reaction, where the feeding solution is added after the translation reaction has incubated for a period of time to make an extended synthesis reaction mixture. The extended synthesis reaction mixture is incubated for an additional period of time to synthesize one or more proteins. Feeding solutions and methods of performing IVPS using feeding solutions are disclosed herein.

When IVPS reactions that include extracts of detergent-treated cells or lysates are assembled, one or more detergents can be present at a lesser concentration in the in vitro synthesis reaction than in the cell extract, or, if detergent is also added to the reaction buffers, the detergent concentration can remain the same or even be higher in the in vitro synthesis reaction than in the extract. In some embodiments of these methods, a detergent is present in the cell lysate or cell extract at a concentration at or above its CMC, and is diluted to below its CMC in the IVPS reaction. In other embodiments of these methods, a detergent is present at or above its CMC in a cell lysate, and even if diluted, remains above its CMC in the IVPS reaction.

The extract can also be dialyzed to reduce the concentration of detergent in the IVPS reaction. While detergents at a concentration above the CMC are theoretically “not dialyzable”, in practical terms, some dilution of a detergent present above its CMC can occur during dialysis through swelling of the dialysis bag and resulting dilution of the detergent in the sample, and, in cases where the dilution within the dialysis bag is to a concentration below the CMC, further dialysis dilution of detergent in the sample.

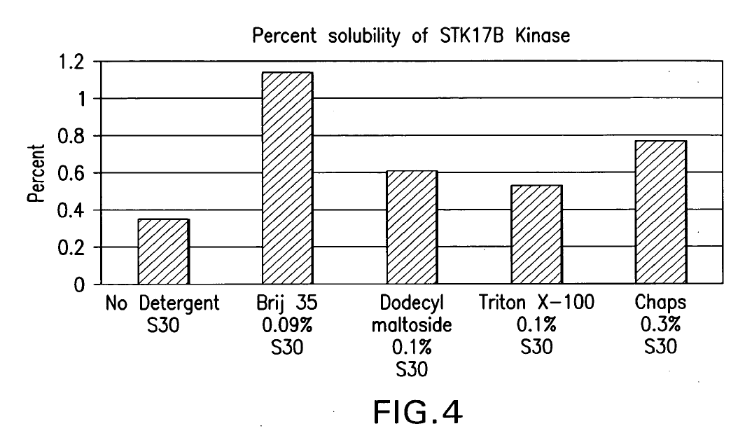

Addition of a detergent to a cell lysis buffer can conveniently treat cell membranes and components with a first concentration of detergent, and subsequently, when the detergent is diluted by addition of the detergent-containing cell extract to an IVPS reaction, provide a lower concentration of detergent in the IVPS reaction. Detergents can be tested for optimal effects on protein synthesis according to their concentration in the lysis buffer. FIG. 4 provides examples of detergents that can be used in the compositions and methods of the invention, their concentrations in a lysis buffer and the resulting extract, and their effects on soluble protein yield. Brij 35 at 0.09%, dodecyl maltoside at 0.1%, Triton X-100 at 0.1%, and CHAPS at 0.3% all enhance the yield of soluble STK17B protein in an IVPS system.

### Methods of Labeling In Vitro Synthesized Proteins for Nuclear Magnetic Resonance (NMR)

The invention also provides methods of labeling proteins with isotopic labels for NMR. The method includes synthesizing a protein in an in vitro protein synthesis system that includes at least one isotopically labeled amino acid, in which a feeding solution is added to the in vitro translation reaction up to one hour after the initiation of the reaction. The method preferably includes the use of a cell extract that has been dialyzed prior to the IVPS reaction for at least 8 hours, with at least one exchange of buffer. More preferably, the cell extract that has been dialyzed prior to the IVPS reaction for at least 2 hours, followed by a dialysis of at least 8 hours, and more preferably yet, the cell extract that has been dialyzed prior to the IVPS reaction for at least 2 hours, followed by a dialysis of at least 12 hours.

For example, the cell extract can be an S30 extract, and the IVPS buffer, and the Feeding solution can be the same as that used for milligram synthesis of proteins as disclosed in Example 2, and the feeding solution disclosed in Table 3, except that isotopically labeled amino acids replace cognate unlabeled amino acids used in the synthesis.

Alternatively, in some cases it can be advantageous to use a an IVPS reaction buffer and feeding solution in which potassium glutamate has been replaced by potassium acetate. The present invention includes methods of making a protein for NMR analysis in an IVPS system, in which the cell extract has been dialyzed for at least eight hours, and the reaction buffer and the feeding buffer include potassium acetate and do not include potassium glutamate.

### Vectors, Dna Cloning and Expression Systems

In some aspects, the invention is drawn to cloning and expression vectors and hosts therefore. The TOPO® cloning system used herein is described in published U.S. Patent Application 2003/0022179 to Chesnut et al., published Jan. 30, 2003, and entitled “Methods and reagents for molecular cloning”, incorporated herein by reference for all disclosure relating to TOPO® cloning systems and methods.

The present invention provides vectors that allow for convenient TOPO®-based cloning of DNA fragments, including but not limited to PCR fragments, provides sequences that promote T7 polymerase-specific transcription of DNA to RNA, and provides sequences that, when transcribed into RNA, enhance translational efficiency of the RNA transcript.

In addition, the vectors include sequences that encode His tags, such that through the transcription and translation process, the peptide tags can be attached to either the N-terminus or the C-terminus of the cloned protein of interest, depending on whether the PEXP5-CT (SEQ ID NO:41) or PEXP5-NT (SEQ ID NO:38) vector is used. Further, the vectors provided in the present invention encode a TEV protease site positioned in the vector to occur between the 6×His tag and the cloned protein of interest. The PEXP5-NT (SEQ ID NO:38) vector construct adds only 21 amino acids onto the N-terminus of the gene of interest and leaves only 2 additional amino acids on the synthesized product after protease (TEV) cleavage. Plasmid pEXP5-CT/TOPO® (SEQ ID NO:41) is designed so that the gene of interest may be inserted with a stop. If no stop codon included, the C-terminal His-tag will be expressed adding 8 additional amino acids to the carboxy terminus of the cloned protein of interest.

### Kits

Kits for in vitro synthesis are also a feature of the present invention. Such kits may contain any number or combination of reagents or components for carrying out the invention. Kits of the invention preferably comprise one or more elements selected from the group consisting of one or more components of the invention (e.g., cell extracts, IVPS reaction buffer, feeding solutions, enzymes, inhibitors, amino acid mixtures or one or more amino acids or derivatives thereof, one or more polymerases, one or more cofactors, one or more buffers or buffer salts, one or more energy sources, one or more nucleic acid templates, one or more reagents to determine the efficiency of the kit or assay for production of the products such as nucleic acid and protein products, and directions or protocols for carrying out the methods of the invention or to use of the kits of the invention and/or its components. The kit of the invention may comprise one or more of the above components in any number of separate containers, tubes, vials and the like or such components may be combined in various combinations in such containers.

In some embodiments the kits of the invention may include at least one extract for protein synthesis, the extract having been made by a method that exposes cells used to make the extract to one or more detergents, surfactants, or lipids prior to lysis, or by a method in which at least one detergent, surfactant, or lipid is added to a cell lysate prior to removal of cell debris from the lysate. The kits can also includes an IVT reaction buffer, amino acids, and a polymerase (such as an RNA polymerase). The kit can also include a feeding buffer.

In some embodiments the kits the kits of the invention may comprise at least one extract for protein synthesis, and a feeding buffer that includes amino acids and at least one energy source. Preferably the cell extract has been made using a phospholipid, detergent, or surfactant added to cells or a cell lysate prior to centrifuging the cell lysate. The kit also preferably includes at least one solution containing one or more amino acids. The kit also preferably includes a polymerase, preferably an RNA polymerase.

The kit can also include: vectors, including the PEXP-CT and -NT vectors disclosed herein, one or more labeled amino acids, and, preferably, instructions for use.

A kit typically includes literature describing the properties of the bacterial host (e.g., its genotype) and/or instructions regarding its use for purifying and/or detecting biomolecules such as His-tagged recombinant polypeptides.

### EXAMPLES

### Example 1

### Compositions of Feeding Solutions

A representative Feeding Solution contains:
(a) a buffer (10-100 mM final concentration);
(b) one or more salts;
(c) one or more reducing agents;
(d) one or more energy sources and/or cofactor;
(e) at least 4 amino acids; and
(f) ammonium acetate

Components of a feeding solution were tested at varying concentrations in in vitro synthesis reactions to optimize protein yield from the reaction.

A. Buffers: HEPES buffer is included to maintain the pH of the reaction. The pH of the feeding solution was increased to pH 8.0 (from 7.6 in the initial reaction). HEPES buffer was included at a concentration such that the final concentration in the in vitro synthesis reaction was preferably from 20-80 mM, where an exemplary feeding solution provided a final concentration of HEPES in the reaction of 57.5 mM. Addition of buffer alone as a feed did not increase yields, but did have a slight stimulatory effect on the activity of the synthesized product, perhaps by allowing better folding of the protein.

B. Salts: The salts included in the feeding solution were identical to those in the initial reaction (to maintain ionic strength), with the exception of the presence of 2 mM CaCl₂. The addition of calcium generated an increase in yields of about 10%.

C. Reducing Agents: Dithiothreitol (DTT) was provided as the reducing agent.

D.1. Energy sources: The glycolytic intermediates tested include phosphoenol pyruvate, acetyl phosphate,glucose-6-phosphate (Glu6-P), fructose-6-phosphate, and 3 phosphoglycerate, individually, or in combination with each other. The optimal total concentration of glycolytic intermediates was found to be 20 mM-60 mM (final concentration in the synthesis reactions).

The addition of any glycolytic intermediates without the cofactor NAD or NADH enhanced enzyme activity, and together with NAD or NADH stimulated both better expression and activity. These effects were observed through a range of concentrations of each component, for example, amino acids 1-5 mM; glycolytic intermediates 5-100 mM, and NADH 0.1-1 mM.

D.2. Co-factors: NAD or NADH was provided in the feeding solution to provide 0.1-1 mm final concentration in Assays.

E. Amino Acids: amino acids were provided in the feeding solution to give a final concentration of 1.25 mM. Final concentrations of up to 5 mM amino acids were not detrimental. Amino acids provided in a feeding solution increase yields up to 30% over the addition of buffer alone (Table 4), probably due to the replacement of some degraded or depleted amino acids with fresh ones. The initial amino acid concentration in the reaction was 1.25 mM for each amino acid (except methionine and cysteine provided at 1.5 mM), and increasing this concentration of amino acids initially did not generate the same spike in yields. Thus, it seems that it is the supplementation at a later time that is important. The current Feeding Solution contains 1.25 mM each amino acid except for methionine and cysteine, which are present at 1.5 mM.

A preferred feeding solution (not including the amino acids that were also provided in the feeding solution at 1.25 mM, except for methionine and cysteine, which are present at 1.5 mM) is described in the following table. The Feeding Solution (minus amino acids) described in Table 3 was prepared and evaluated in IVPS reactions as described in the following Examples.

TABLE 3
FEEDING SOLUTION

| | 2X Concentration | 1X Concentration |
|---|---|---|
| Component | (mM) | (mM) |
| 1M HEPES-KOH, pH 8.0 | 115 | 57.5 |
| 1M DTT | 3.4 | 1.7 |
| 3M K Glutamate | 460 | 230 |
| 2M MgOAc | 28 | 14 |
| 7.5M NH₄OAc | 160 | 80 |
| 1M CaCl₂ | 4 | 2 |
| 1M Glu-6-P | 90 | 45 |
| 100 mM NAD | 1.0 | 0.5 |
| 20 mg/ml Folinic Acid | 68 micrograms/mL | 34 micrograms/mL |
| 100 mM cAMP | 1.3 | 0.65 |

### Example 2

### Yields and Activity with Various Components in the Feed Solution

Standard 50 microliter Expressway™ Plus (Invitrogen, Carlsbad, Calif.) reactions were assembled and incubated at 37° C. essentially according to the manufacturer's instructions. The reactions included 600-800 micrograms of E. coli extract made from an RNase A minus mutant and containing 2.5 micrograms per mL of Gam protein, 820U T7 Enzyme, 20U RNase Out, 1 mM amino acids (except methionine) 1.5 mM Methionine, and 0.5-1μg template DNA (either circular or linear) in 1×IVPS Buffer (58 mM Hepes, pH 7.6, 1.7 mM DTT, 1.2 mM ATP, 0.88 mM UTP, 0.88 mM CTP, 0.88 mM GTP, 34 micrograms per mL folinic acid, 30 mM actetyl phosphate, 230 mM potassium glutamate, 12 mM Magnesium Acetate, 80 mM NH₄OAc, 0.65 mM cAMP, 30 mM phosphoenolpyruvate, 2% polyethylene glycol). The reactions were performed in 1.5′-2 ml microfuge tubes in an Eppendorf Thermomixer at either 30° C. or 37° C. with moderate shaking (1000-1400rpm) for 2-6 hours. Reactions were fed with one-half volume (with respect to initial reaction volume) of feed buffer at different intervals over the reaction period.

In assays testing the effect of detergents, detergents were included in the S30 buffer in which the cells were lysed, and were present in the reaction at varying concentrations: octylglucopyranoside (0.6%, 1.2%, 2%), octylthioglucopyranoside (0.3%, 0.6%, 0.9%), Zwittergent® 3-14 (0.01%, 0.025%, 0.05%), sodium dodecyl maltoside (0.01%, 0.025%, 0.05%), and Triton® X-100 (0.01%, 0.025%, 0.05%). Each detergent was included in the reaction at three concentrations corresponding to below the critical micelle concentration, at the critical micelle concentration, and above the critical micelle concentration for that detergent.

The reactions were prepared with 1 μg of plasmid DNA or 2-3 μg of linear templates. Plasmids used as DNA templates were pEXP1-LacZ, pCR2.1-GFP (Green Fluorescent Protein), and pEXP3-GUS.

Feeding solutions containing components indicated were fed to the reaction at 30 minutes and 2 hours in 25 microliter volumes. Feeding Solutions contained 58 mM HEPES-KOH pH 8.0, 230 mM Potassium Glutamate, 12 mM Magnesium Acetate, 80 mM Ammonium Acetate, 2 mM Calcium Chloride and 1.7 mM DTT. The feed may also have contained amino acids at 1 mM each (except for Methionine at 1.5 mM), arid/or glycolytic intermediates such as Glucose-6-Phosphate, 3-Phosphoglycerate (3-PGA) or Acetyl Phosphate (AP) at 30 mM, and NADH at 0.3 mM. The amount of GFP synthesized and the activity (Relative Fluorescence Units, RFU) was determined (Table 4). All the Reaction Feeding Solutions described below result in higher yields than the “no feed” or “buffer only” controls. The feeding solution comprising amino acids, Glu-6-P and NADH performed best; the next best performance was seen in the feeding solution comprising amino acids, 3-PGA, and NADH.

Protein yields for a panel of control proteins synthesized in a four to six-hour reaction that received feeds at 30 min. and at 2 hr., in which the feeding solution contained amino acids and an energy source, consistently yielded greater than one mg/ml of protein.

TABLE 4
EXPRESSWAY REACTIONS WITH FEEDING SOLUTION

| | Yield | Activity |
|---|---|---|
| Reaction Feeding Solution | ug GFP | RFU |
| 50 μl rxn no feed | 26 | 5996 |
| +Buffer only | 25 | 6994 |
| +amino acids | 39 | 8981 |
| +amino acids, Glu-6-P | 42 | 15212 |
| +amino acids, Glu-6-P, NADH | 58 | 21713 |
| +amino acids, 3-PGA | 35 | 16467 |
| +amino acids, 3-PGA, NADH | 49 | 21375 |
| +amino acids, AP | 32 | 14466 |
| +amino acids, AP, NADH | 43 | 17369 |
| 100 ul rxn (scale-up) | 50 | 11991 |

### Example 3

### Effect of Feeding Times and Volumes on Expression of LacZ and GFP

Standard 50 microliter Expressway™ Plus reactions were assembled as described in Example 1 and incubated at 37° C. Feeding buffer was added at the time indicated. For single time feeds, a 1 volume feed (50 μl) was added, for dual feeds, two volume feeds were added (25 μl each). Total protein yield was calculated based on [³⁵S]-Methionine incorporation. LacZ activity was determined using a luminescent assay and is reported as Relative Luminescent Units (RLU). GFP activity was determined by its fluorescent emission (excitation: 395 nm; emission: 509 nm) and is reported as Relative Fluorescent Units (RFU).

In the case of single feeds, for both proteins, there was an increase in activity when the feeding solution was added at 15 min, 30 min, 1 hr, or 2 hr after the reaction was initiated (Table 5). Amount of protein synthesized, however, was optimal when the feed occurred at 15 min, and also improved yields when it occurred at 30 min and 1 hr. The effect of a single feed at 2 hr on yield was much less than the effect of earlier single feeds.

In the case of double feeds, for both proteins, there was an increase in activity when the Feeding Solution was added twice, at 0 and 2 hr; 15 min and 2 hr; 30 min and 2 hr; 1 hr and 2 hr; and 1 hr, 3 hr. As in the case of single feed reactions, providing a first feed later than the 1 after the reaction was initiated had much less of an effect on protein yield than earlier (15 min, 30 min, 60 min) first feeds.

TABLE 5
EFFECT OF FEEDING TIMES AND VOLUMES

| | | | | |
|---|---|---|---|---|
| | μg LacZ | Activity | μg GFP | Activity |
| Feed Time | synthesized | (RLU) | synthesized | (RFU) |

Controls

| | | | | |
|---|---|---|---|---|
| No Feed | 48 | 216168 | 48 | 7530 |
| 0 min | 72 | 358427 | 84 | 21379 |

Single Feeds

| | | | | |
|---|---|---|---|---|
| 15 min | 80 | 481205 | 85 | 21048 |
| 30 min | 65 | 534895 | 78 | 21306 |
| 1 hr | 59 | 767759 | 80 | 17483 |
| 2 hr | 49 | 508980 | 63 | 18256 |

Double Feeds

| | | | | |
|---|---|---|---|---|
| 0, 2 hr | 70 | 449821 | 86 | 19697 |
| 15 min, 2 hr | 72 | 441122 | 93 | 19804 |
| 30 min, 2 hr | 69 | 469806 | 97 | 18514 |
| 1 hr, 2 hr | 61 | 612933 | 76 | 18932 |
| 1 hr, 3 hr | 50 | 463709 | 78 | 14516 |

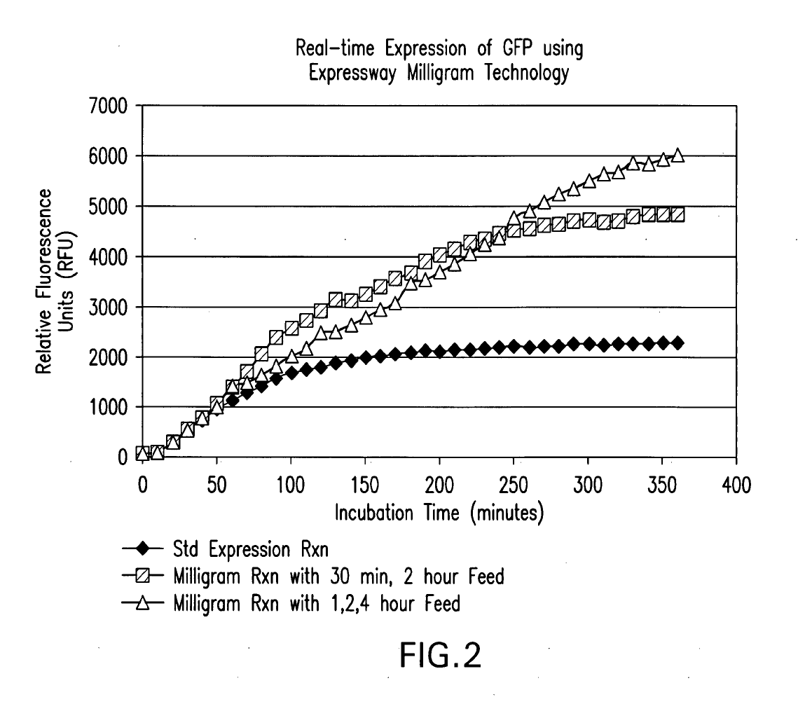

In a similar experiment, fluorescence activity of Green Fluorescent Protein (GFP) was monitored during the in vitro expression reaction. A series of 50 μl reaction mixtures were prepared and fed with indicated volumes of feeding buffer at various times. The reactions were performed for 6 hours at 37° C. with intermittent shaking. GFP activity was monitored over 6 hours of incubation in a Spectramax Gemini Fluorometer. The results are shown in FIG. 2. Standard in vitro reactions (diamonds) stop almost completely after 2 hours. With Expressway-Milligram Feeding technology, the reaction continues almost linearly for 6 hours with either the addition of feeding buffer (1) at 30 min. and again at 2 hrs (squares) or (b) at 1 hr and again at 2 hr and 4 hr (triangles).

### Example 4

### Synthesis of Milligrams of Proteins

The following human ORFs were cloned into pEXP1 or pEXP3 using Gateway technology (Invitrogen, Carlsbad, Calif., see U.S. Pat. Nos. 5,888,732 and 6,277,608, both herein incorporated by reference for all disclosure relating to Gateway cloning technology, methods, and vector systems), and pEXP5-NT/TOPO® (SEQ ID NO:38) and pEXP5-CT/TOPO® (SEQ ID NO:41) through TOPO® TA cloning (Invitrogen, Carlsbad, Calif., see U.S. Pat. Nos. 5,851,808 and 6,828,093, both hereby incorporated by reference for all disclosure relating to TOPO® cloning technology, methods, and vector systems): Brain creatine kinase B-chain (CKB; Invitrogen catalog # IOH5211; Genbank NM 001823); Major histocompatibility complex, class II, DO alpha; HLA-D0-alpha; (HLA-DOA; Invitrogen catalog # IOH10959; Similar to creatine kinase, muscle (CKM; Invitrogen catalog # IOH7287; Genbank NM 001823); Calmodulin-like 3 (CALML3; Invitrogen catalog # IOH22362; Genbank NM 005185); and Interleukin 24 (IL24; Invitrogen catalog # IOH9846 Genbank BC009681).

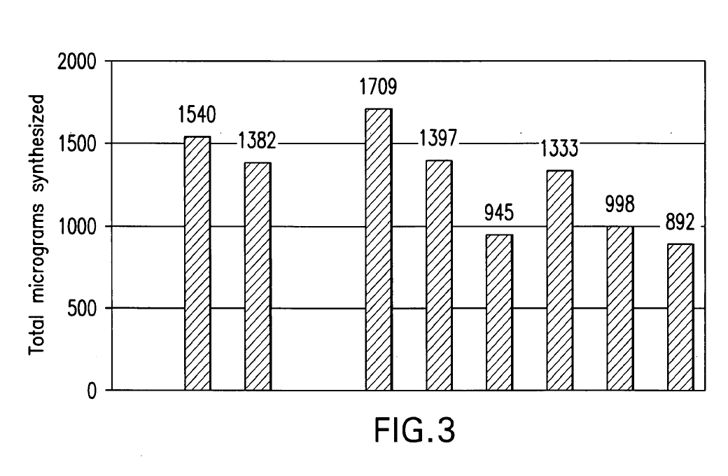

In vitro protein synthesis reactions were performed using the initial reaction conditions given in Example 2, and the Feeding Solution provided in Table 3, except that the initial reaction volume was 1 mL, and a single feed of one volume (1 mL) of feeding solution was performed at 30 minutes. Representative yields of 8 proteins were determined by ³⁵S-Methionine incorporation. In these experiments, the initial reactions contained 5(Ci of [35S] methionine (10 μCi/μl, 1175 Ci/mmol). The feed buffer was supplemented with and [35S] methionine at the same ratio as in the base (starting) reaction (0.5(Ci of (10 μCi/μl, 1175 Ci/mmol) per 50 microliters). Several 0.25 (1 aliquots of these Expressway™-Milligram reaction products were electrophoresed on a Coomassie Blue stained 4-12% NuPAGE® gel and determination of the yield of each of 8 proteins was carried out. FIG. 3 shows the amount of each protein synthesized in this Expressway™-Milligram system. The expressed proteins are, from left to right, GFP; human ORF Brain Creatine Kinase; LacZ; 6-human ORF MHC class II; human ORF Creatine Kinase muscle in an N-terminal his tag vector (pEXP5-NT/TOPO® (SEQ ID NO:38)); human ORF Calmodulin Like 3; human ORF Interleukin 24; and human ORF Creatine Kinase muscle in a C-terminal his tag vector (pEXP5-CT/TOPO® (SEQ ID NO:41).

The range of protein produced among the 8 proteins was roughly 890 to 1,700 mg. Of the 8 proteins, 5 were produced in quantities >1.3 mg, and 2 of the 8 proteins were produced in quantities >1.5 mg. The average (mean) amount of protein produced was 1.275 mg.

### Example 5

### Ivps Extracts from Bacterial Cells

The following protocols are used to prepare S30 extracts from bacterial strains, including E. coli.

### Cell Paste

E. coli K12A19 cells were grown in 50-L Buffered 2× YT (Tryptone, 16 g/L; Yeast Extract, 10 g/L; Sodium chloride, 5 g/L; Dibasic sodium phosphate anhydrous Na₂HPO₄, 5.68 g/L; Monobasic sodium phosphate anhydrous Na₂HPO₄, 2.64 g) supplemented with Cerelose (5 g/L). Cells were incubated at 37° C. on a rotating platform (typically, 250 rpm), until the OD₅₉₀ reached a range of from about 3.0 to about 5.0, which typically took from about 6 to about 8 h. The cells were freshly inoculated into fresh media with a starting OD₅₉₀ of about 0.05 to about 0.10, and then incubated at 37° C., at 250 rpm, 50 slpm, 5 psi, to an OD₅₉₀ of from about 3.0 to about 3.5. Cells were transferred to Sorvall GS3 bottles and centrifuged for 15 min at 5000×g. The supernatant was removed, with aspiration if needed. (The cell paste can be stored, preferably for 5 days or less, at −80° C. before proceeding to the next step.)

One gram of cell paste, thawed first if stored at −80° C., was resuspended in 1 ml of chilled (4° C.) S30 buffer with DTT added immediately prior to use (for example, 250 ml S30 buffer for 250 g cells).

The cells were swirled gently by hand for a few minutes (without generating froth) to hasten the resuspension process. A sterile stir bar was placed into a bottle containing cells and was stirred gently for approximately 15 min to completely resuspend cells. The resuspension was placed on ice immediately.

### Cell Lysis

Before cell lysis, cells were washed with S30 buffer+6 mM beta-mercaptoethanol. This was carried out by adding S30 buffer, 6 mM beta-mercaptoethanol to cells and “mashing and stirring” with a 25 ml pipette until the cell paste was dissolved. The S30 Buffer was 10 mM Tris, 14 mM magnesium acetate and 60 mM potassium actetate, pH 8.2. The suspension was spun in an RC3B centrifuge for 20 minutes at 4,500 rpm. The supernatant was decanted, and the wash was repeated.

The cells were resuspended in a 0.85 to one ratio (0.85 ml buffer: 1 g pellet) of 1×S30 Buffer, 1 mM DTT, 0.5 mM PMSF, and 0.1% Triton X 100. In some cases, no detergent or different detergents were added to the lysis buffer, such as Brij 35 (0.09%), dodecyl maltoside (0.1%), and CHAPS (0.3%).

A 5 ml sample of resuspended cells was placed into 995 ml water (1:200 dilution) to determine a starting OD. This sample was vortexed and read at 590 nm using water as a blank. Immediately before proceeding to cell disruption, 0.1 M Phenylmethanesulfonyl fluoride (PMSF) was added to the resuspended cell paste. Five (5) μl of 0.1 M PMSF per ml of cell suspension was used.

An Emulsiflex C50 homogenizer (Avestin Inc., Ottawa, Canada) was used to disrupt the cells. Pressure was kept at from least about at 25,000 to about 30,000 psi. It generally took approximately 15-20 mM to pass 500 ml cell suspension through the homogenizer.

If was the efficiency of lysis was less then about 90%, the cell suspension is passed through the homogenizer again. The efficiency of lysis was calculated as follows (First Pass OD590/initial OD590, see above)×100=% not lysed; 100−% not lysed=% efficiency of lysis.

One (1) M DTT was immediately added to lysate to a final concentration of 1 mM (e.g., 250 microliters of 1 M DTT per 250 ml lysate). The lysate was then centrifuged at 16,000 rpm (30,000×g) in an SS34 rotor for 40 min at 4° C. The upper four-fifths of supernatant was removed with a sterile plastic graduated pipet and collect in a sterile 1 L container.

### Preincubation

The volume of supernatant was measured. 10× Preincubation Mix was added to the supernatant (after 2 post-lysis centrifugations) to a final concentration of 1× and the lysate was incubated at 37 degrees C. for 150 minutes. The preincubation mix was prepared just before use by adding the components in the order listed below. It was kept on ice

TABLE 6
PRE-INCUBATION MIX

| 10X Conc. | | Component |
|---|---|---|
| 0.73M | | Tris-Acetate, pH 8.2 at 22° C. |
| 23.2 | mM | Magnesium Acetate |
| 33 | mM | ATP pH 7.0 |
| 225 | mM | Phosphoenol Pyruvate pH 7.0 |
| 11 | mM | DTT |
| 100 | μM | Amino Acid Mix (-Met) |
| 100 | μM | Methionine |
| 1260 | units | Pyruvate Kinase |

Preincubation mix, 1× concentration:

73 mM Tris-acetate, pH 8.2 at 22 C

10 μM amino acid mix

1.1 mM Dithiothreitol (DTT)

2.3 mM Magnesium Acetate

21 mM Phosphoenol Pyruvate

60 mM Potassium Acetate

3.3 mM ATP

126 U/ml Pyruvate Kinase

The mixture was incubated in a 37° C. shaking water bath, shaking gently at 150 rpm for 150 min. The extracts were then dialyzed against 1×S30 Buffer containing the same concentration as in the S30 buffer used to resuspend the cells, plus 1 mM DTT, with two exchanges overnight. The extract was then aliquoted and stored at −80 degrees C.

### Example 6

### Comparison of Soluble Protein Yield Using Ivps Extracts Made with Different Surfactants

S30 extracts were prepared as described in the preceding example, except that bacterial cell pellets were resuspended with S30 buffer containing either no detergent, or one of the following detergents: 0.09% Brij 35, 0.1% Dodecyl maltoside, 0.1% Triton X-100, or 0.3% Chaps. All detergents were used at concentrations above the CMC. The resuspended cells were then lysed in the C5 Emulsiflex. The lysed cells were centrifuged as described above, and the supernantant was pre-incubated with 1/10 vol of translation mix and pyruvate kinase. The extracts were then dialyzed against S30 buffer (without detergent) with two exchanges overnight.

IVT reactions were performed as described in Example 2, where a single feed was provided at 30 minutes using the Feeding Solution provided in Table 3. The template was a plasmid encoding the STK17B Kinase protein (serine threonine kinase 17b; Invitrogen catalog # IOH21114; Genbank NM 004226.2) using extracts prepared with different detergents. FIG. 4 shows that providing detergent during lysis of cells improved the solubility of the STK17B Kinase protein compared to the S30 prepared without detergent. It is notable that both nonionic (Brij 35, Dodecyl maltoside, Triton X-100) and zwitterionic (Chaps) detergents enhanced soluble protein yield.

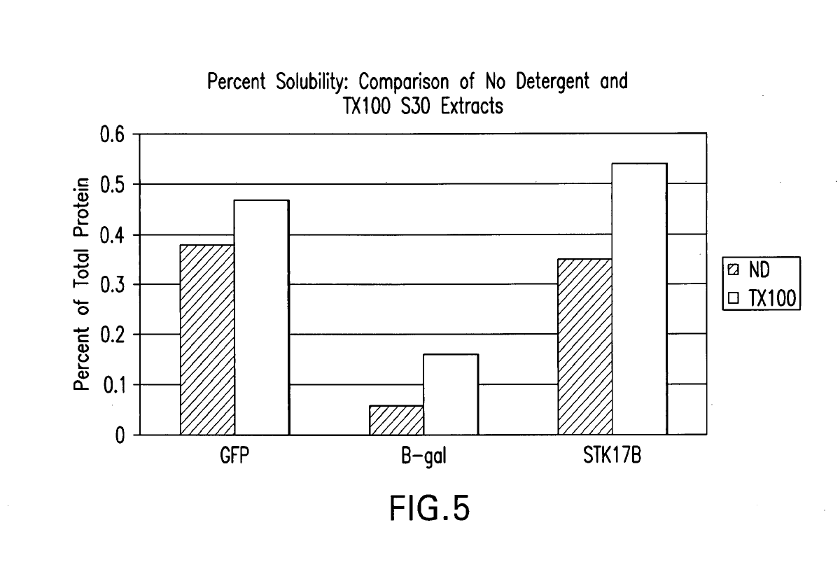

In another set of experiments, the effects of using S30 buffer containing 0.1% Triton X 100 for resuspending cells on the soluble yields of several proteins was tested. FIG. 5 shows the soluble yields of GFP, LacZ, and the STK17B Kinase protein when translated using an extract that was made with and without detergent (Triton X-100) present during the lysis of cells. In all three cases, the yield of soluble protein is enhanced by the presence of detergent in the S30 buffer.

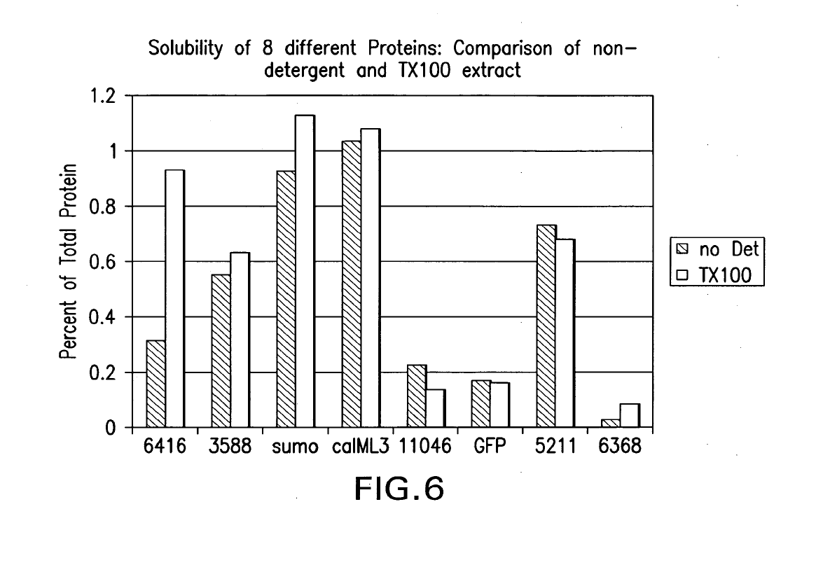

FIG. 6 shows enhanced solubility of a range of proteins synthesized in S30 extracts prepared with 0.1% Triton X-100 in the S30 buffer in which cell were lysed, including (from right to left) CDC28 protein kinase regulatory subunit 1B (CKS1B; Invitrogen catalog # IOH6416; Genbank NM_{—}001826); syntaxin binding protein 1 (STXBP1; Invitrogen catalog # IOH3588; Genbank BC015749.1); Sumo protein (SEQ ID NO:1); Calmodulin-like 3 (CALML3; Invitrogen catalog # IOH22362; Genbank NM 005185); Adenylate Kinase 3 alpha like (AK3L1; Invitrogen catalog # IOH11046; Genbank NM_{—}016282); GFP; Brain creatine kinase B-chain (5211; Genbank NM 001823); and Receptor-Interacting Serine/Threonine Kinase (6368; Genbank NM 003821).

### Example 7

Effects of Adding Detergent Extracts of Cellular Membranes to In Vitro Translation Systems

To determine whether treating cells or cell lysates with one or more surfactants or detergents prior to separating cellular membranes from the cell extract to be used for translation has beneficial effects on the yield or activity of synthesized proteins, “add back” experiments were performed. In the following experiment, cells were lysed and cell extracts were made (using centrifugation) in the absence of detergent. Lysed cell pellets were separately extracted with detergent lysed cell pellet extracts and aliquots of the pellet extracts were added back to S30 extracts that were used in PITT reactions to determine whether components that could be extracted from the cell pellet fraction of the preparation with detergent could have a beneficial effect on in vitro translation.

Bacterial (E. coli A19) S30 extracts were made according to standard procedures. Briefly, washed E. coli cells were resuspended in S30 Buffer (10 mM Tris, 14 mM magnesium acetate and 60 mM potassium actetate, pH 8.2), and lysed in an Emulsiflex C50 homogenizer (Avestin Inc., Ottawa, Canada). The lysate was centrifuged. The S30 supernatant was removed and aliquoted.

The S30 pellet from 1 liter of cell culture was resuspended in 10 milliliters of buffer (20 mM KHPO4, pH 8, 150 mM KCl). Five hundred (500) microliter aliquots of the resuspended S30 pellet were distributed into 1.5 mL tubes on ice. Either 50 or 100 microliters of each of a number of nonionic and zwitterionic detergents (sodium deoxycholate, sodium dodecyl maltoside, digitonin, octyl thioglucoside, octyl glucoside, or CHAPs) were added to the S30 fractions to a final concentration of 0.5% and the fractions plus detergent were incubated at room temperature for 30 minutes. Controls received 100 microliters of buffer (20 mM KHPO4, pH 8, 150 mM KCl) only or 0.5 M NH4OAc. The samples were then centrifuged in a microcentrifuge for 30 mM. and 14,000×g. The supernatants were transferred to clean tubes.

Ten microliters of each S30 pellet detergent extract supernatant were loaded on an SDS PAGE gel. The gel was electrophoreses and stained with Coomassie blue. Visual analysis of the stained gel showed that in lanes of the gel having S30 pellet detergent extracts, there were high molecular weight (greater than 120 kilodalton) stained bands and diffuse material in lanes that were not observed in lanes that had been loaded with the supernatants of buffer or salt-incubated S30 pellets.

The extracts were also used in IVTT reactions. Standard 50 microliter reactions were performed using 20 microliters of 2.5 × IVT buffer (145 mM HEPES-KOH, pH 7.6, 4.25 mM DTT, 3.0 mM ATP, 2.2 mM UTP, 2.2 mM CTP, 2.2 mM GTP, 85 micrograms per milliliter folinic acid, 75 mM acetyl phosphate, 575 mM potassium acetate, 30 mM magnesium acetate, 200 mM NH4OAc, 1.625 mM cAMP, 75 mM PEP, 5% PEG), amino acids at 1.25 mM final concentration (except met and cys, both at 1.5 mM) 17 microliters of S30 pellet extract, having 0.25 microliters of ³⁵S methionine, 0.75 micrograms of pucT7-GFP plasmid DNA, 1 microliter of T7 polymerase, and 3 microliters of S30 pellet extracts. The pellets had been treated with either 0.5% sodium deoxycholate, 0.5% sodium dodecyl maltoside, 0.5% digitonin, 0.5% octyl thioglucoside, 0.5% octyl glucoside, or 0.5% CHAPs. The final detergent concentration in the translation reactions was 0.03% in each case. As a control, an IVTT reaction was performed with 3 microliters of S30 pellet extacts in which the pellet fraction had been inclubated with 0.5M NH4OAc, in place of detergent. As further controls, 3 microliters of 2.5× IVS buffer or 3 microliters of additional S30 extract were added to reactions.

The reactions were performed in 1.5-2 ml microfuge tubes in an Eppendorf Thermomixer at 37(C with moderate shaking (1000-1400rpm) for 2 hours.

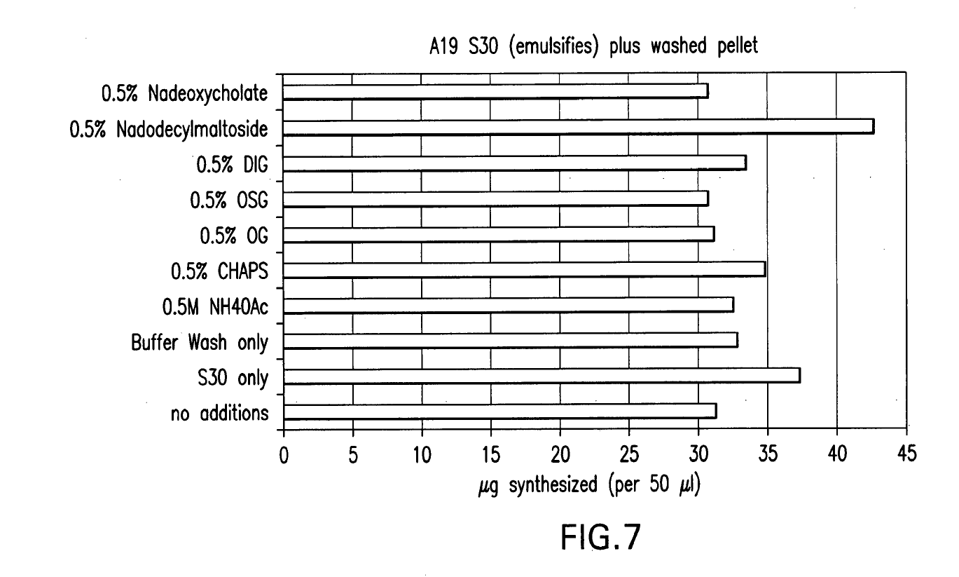

Active GFP protein yield was assessed by monitoring GFP fluorescence over time. Protein yield was determined by 35S methionine incorporation. FIG. 7 shows that when using extracts from the E. coli A19 strain, the addition of an S30 pellet detergent extract to the IVPS reaction results in an increase the amount of protein synthesized.

### Example 8

Incorporation of 15N-Labeled Cell-Free Amino Acids into In Vitro Expressed Proteins

The ¹⁵N-labeled SUMO protein (SEQ ID NO:1; U.S. Pat. No. 6,872,343, herein incorporated by reference for all disclosure related to SUMO protein and nucleic acid sequences and their uses) was purified and was examined by mass spectrometry to determine the extent of incorporation of ¹⁵N. Ideally, the protein of interest should have no unlabeled amino acids; a homogenous population of labeled protein is desired for applications such as NMR. The Mass Spec results comparing the tracings for control (unlabeled) SUMO protein to ¹⁵N-labeled SUMO protein are shown in FIG. 4. The results show complete, or nearly complete, incorporation of ¹⁵N-labeled amino acids to the exclusion of any natural, unlabeled amino acids.

### Expression and Purification of CALML3 in Expressway™ NMR

SUMO and the human ORF Calmodulin Like 3 (CALML3), were cloned into the pEXP5-NT/TOPO® TA vector and expressed in 5 ml Expressway™ NMR reactions containing 10 mg/ml uniform labeled ¹⁵N amino acids. The SUMO and CALML3 proteins were synthesized and purified as described above. The final reactions were diluted 1:1 with binding buffer, purified over Ni-NTA resin, and the purification profile was analyzed on a Coomassie stained 4-12% NuPAGE® gel. Peak fractions were combined and dialyzed before mass spectroscopy analysis. The final recovery was approximately 4 mg of SUMO and 4.5 mg purified CALML3.

### Labeling of SUMO Protein

For each IVTT reaction following components were added and incubated at 30° C. for 4 hours; 20 ml E. coli S30, 20 ml 2.5× IVPS NMR Buffer (145 mM HEPES-KOH, pH 7.6, 4.25 mM DTT, 3.0 mM ATP, 2.2 mM UTP, 2.2 mM CTP, 2.2 mM GTP, 85 micrograms per milliliter folinic acid, 75 mM acetyl phosphate, 575 mM potassium acetate, 30 mM magnesium acetate, 200 mM NH4OAc, 1.625 mM cAMP, 75 mM PEP, 5% PEG; no amino acids in the buffer), 1 ml T7 RNA Polymerase, 0.5 ml RNaseOUT™, 5 ml of 100 mg/ml ¹⁵N-labeled cell free amino acids (final concentration in the reaction was 10 mg/ml), 1 mg DNA, 0.5 ml ³⁵S-Methionine and made to 50 ml with nuclease free H₂O. Fifty (50) ml of Feeding buffer (25 ml 2× feeding buffer, 0.5 ml ³⁵S-Methionine, 5 ml of 100 mg/ml ¹⁵N-labeled cell-free amino acids and made to 50 ml with nuclease free H₂O) was added to each reaction at 30 minutes. At the end of the 4 hours, 5 ml of reaction was used for TCA precipitation to determine the yield of protein. For the unlabeled protein expression, the labeled amino acids were replaced with non-labeled amino acids (1 mM in the final reaction) and carried out the IVTT reactions as mentioned above.

For labeling using ¹³C or ¹⁵N labeled amino acids, the appropriate non-labeled amino acids were replaced with the labeled ones. For labeling using the complete amino acids (including ¹³C or ¹⁵N labeled), 100 mg/ml were dissolved in 50 mM HEPES, pH 7.5 and a 5 μl aliquot was used in a 50 μl IVTT reaction. The amino acid concentration was 10 mg/ml in the final reaction. The concentration can be changed as preferred by the researcher.

50 μl of 10 mM of the mixture of all 20 amino acid was added to 0.25 ml of 2× Feeding buffer with and brought the volume to 0.5 ml with nuclease free water.

For a 0.5 ml of Expressway NMR reaction, following components were mixed; 0.2 ml E. coli S30, 0.2 ml 2.5× NMR IVPS Buffer, 10 μl T7 RNA polymerase Mix, 50 μl 10 mM amino acid mix (labeled amino acid mixture or unlabeled amino acid mixture), 2.5-5 μg of DNA template (circular or linear). Then the final volume of the mixture was brought to 0.5 ml with nuclease free water. The reactions were incubated at 30° C. or 37° C. for 4 hours. After 15-30 minutes from the start of the reaction 0.25 ml of Feeding buffer was added to the IVTT reactions. Then after 2 hours, 0.25 ml of Feeding buffer was added to the reaction and incubated at 30° C. or 37° C.

Following the reaction, an aliquot of 5 μl of the reaction was incubated at room temperature for 5 minutes with 100 μl N NaOH. Then 10% of cold TCA (trichloro acetic acid) was added and kept at 4° C. for few minutes. The precipitated protein was collected to glass fiber filters (GF/C) using a vacuum manifold. The filters were washed twice with 5% TCA and finally with 100% ethanol. Dried filters were transferred to scintillation vials filled with scintillation liquid. The protein yield was calculated by ³⁵S methionine incorporation of TCA precipitable counts.

After completion of the IVTT reaction, the reactions were centrifuged at 16000×g for about five minutes. The samples were diluted in binding buffer 1:1 and loaded on to an appropriate amount of Ni-NTA columns that had been equilibrated with binding buffer. The samples were incubated in the column for about 5 minutes and the flow-through was collected. Then the column was washed with 10 column volumes of wash buffer and collected the first half as wash 1 and 2^{nd} half as wash 2 (5 column volumes each). One column volume of Elution buffer 1 was added to the column and eluted the non-specific proteins bound to the column. One column volume of the elution buffer 2 was added to the column and incubated for about 3-5 minutes and the proteins were eluted, repeated the elution again with one column volumes of elution buffer 2. After all specifically bound proteins have been eluted from the column; the column was washed with elution buffer 3. The samples were run on a NuPAGE gel. The elutions containing protein were pooled and dialyzed against the dialysis buffer for few hours. The concentration of the protein was determined using the Bio-Rad Assay reagent, and the percent incorporation of labeled amino acids was determined by Mass Spectroscopy.

To remove the MALDI-TOF non-compatible buffer components such as NaCl and DTT, the samples were buffer-exchanged against 0.1% TFA (trifluoro acetic acid) using drop dialysis technique. The buffer exchanged samples were then analyzed on a VOYAGER-DE-STR MALDI/TOF instrument (ABI, Foster City, Calif.) using supersaturated Sinapinic acid dissolved in 50% Acetonitrile/0.1% TFA as matrix. The samples were calibrated both internally and externally against Invitromass-IV and Invitromass-30 kDa.

Samples were digested in 20 mM ammonium bicarbonate pH 8.0 with 10 ng/ml trypsin (sequencing grade modified trypsin, Promega) for 30 mM at 37° C. After proteolysis, the samples were concentrated by C18 reverse phase extraction using Zip-Tips (Millipore). The eluate was deposited onto a stainless-steel MALDI-TOF-MS sample target and mixed 1:1 with Maxlon AC MALDI matrix (Invitrogen).

MALDI-TOF-MS analysis was performed using an Applied Biosystems Voyager DE STR instrument. All MALDI-TOF-MS spectra were acquired in the positive reflectron mode (unless specified) with acceleration voltage at 20 kV, delay time 50-250 nsec, 300 laser shots per spectrum, laser intensity 1500-1700, digitizer vertical scale set at 500 mV. Spectra were calibrated externally or internally using the InvitroMass LMW calibrant kit (Invitrogen). The peptide mass fingerprints of digested calmodulin and SUMO proteins were analyzed by Voyager Explorer software (Applied Biosystems). Quantitation of heavy-isotope incorporation was calculated using Isotope Ratio Calculator (ChemSW).

MALDI-TOF-MS analysis of intact proteins was performed using an Applied Biosystems Voyager DE STR instrument. Analysis was performed with a constant laser intensity setting of 1998 in the linear mode. Samples were mixed 1:1 (v/v) with a saturated solution of Sinapinic acid (Sigma) in 50% acetonitrile 0.1% TFA (Pierce).

$$Calculation of Percent Incorporation - Incorporation = \frac{Intensity of Labeled peak}{( Intensity of control peak + Intensity of labeled peak )}$$

$$The percent incorporation of {}^{15} N Arginine of labeled peptide = \frac{100}{( 25 + 100 )} = 80 \%$$

2.5× IVPS NMR Buffer with Potassium Acetate or Potassium Glutamate

One of the components in the IVPS buffer, potassium glutamate, helps high expression of proteins. Potassium glutamate in the system releases free glutamic acid to the reaction; glutamic acid is a precursor for glutamine and aspartic acid. The presence of this compound makes it difficult to label the protein with ¹³C/¹⁵N Aspartic acid, ¹³C/¹⁵N Asparagine, ¹³C/¹⁵N Glutamic acid or ¹³C/¹⁵N Glutamine. To overcome this problem, we have tried several alternatives to replace the potassium glutamate without reducing the protein yield. Most of the compounds we tried significantly reduced the protein yields. Potassium acetate was one of the choices to replace the potassium glutamate, but the protein yields were 50% lower than the potassium glutamate.

### Glutamate-Based System

Two SUMO peptides were examined for the extent of incorporation of stable isotopes therein when the potassium glutamate-based buffers are used. ¹⁵N-Glu

The SUMO 47-54 peptide (mass peak 965.4) comprises one Glu residue in its amino acid sequence:

| | |
|---|---|
| Arg-Leu-Met-Glu-Ala. | (SEQ ID NO: 2) |

No incorporation of ¹⁵N Glu was detected (0% inc) in the presence of glutamate.

### ¹⁵N-Gln

The SUMO 72-109 peptide (mass peak 4229.1) comprises three Gln residues in its amino acid sequence:

(SEQ ID NO: 3)

Ile-Gln-Ala-Asp-Gln-Thr-Pro-Glu-Asp-Leu-Asp-Met-
Glu-Asp-Asn-Asp-Ile-Ile-Glu-Ala-His-Arg-Glu-Gln-
Ile-Gly-Gly-Pro-Gly-Gly-Gly-Ser-His-His-His-His-
His-His.

No incorporation of ¹⁵N Gln was detected (0% inc) in the presence of glutamate.

### Acetate-Based System

CALML3 peptides were examined for the extent of incorporation of stable isotopes therein when the potassium acetate-based formulations are used.

### ¹⁵N-Glu

The 47-54 CALML3 peptide (mass peak 965.5242) comprises 1 Glu residue in its amino acid sequence:

| | |
|---|---|
| Gly-Cys-Ile-Thr-Thr-Arg-Glu-Leu. | (SEQ ID NO: 4) |

When ¹⁵N-Glu was used in the reactions, part of the normal peak (m/z=965.5242) was “shifted” one Dalton (+1 Da) (m/z=966.5238) due to the incorporation of 15N. The percent incorporation was 35% (35% inc) in the absence of glutamate.

The 48-55 CALML3 peptide (mass peak 966.5380) comprises 1 Glu residue in its amino acid sequence:

| | |
|---|---|
| Cys-Ile-Thr-Thr-Arg-Glu-Leu-Gly. | (SEQ ID NO: 5) |

When ¹⁵N-Glu was used in the reactions, part of the normal peak (m/z=966.5380) was “shifted” one Dalton+1 Da shift (m/z=967.5344). The percent incorporation was 35% (35% inc) in the absence of glutamate.

### ¹⁵N-Gln

The CALML3 56-64 peptide (mass peak 1063.521) comprises 1 Gin residue in its amino acid sequence:

| | |
|---|---|
| Thr-Val-Met-Arg-Ser-Leu-Gly-Gln-Asn. | (SEQ ID NO: 6) |

When ¹⁵N Gln was used in the reactions, part of the normal peak (m/z=1063.521) was “shifted” one Dalton (+1 Da) (m/z=1064.5759) due to the incorporation of ¹⁵N. The percent incorporation was 65% (65% inc)) in the absence of glutamate.

### Dialysis of S30 Extract

To evaluate the labeling efficiency, both Sumo and CALML3 proteins were labeled with several ¹⁵N labeled amino acids. The Sumo protein was synthesized with short dialyzed S30 (S30 dialyzed for two hours) using ¹⁵N Asparagine, ¹⁵N Glycine, ¹⁵N Tyrosine, ¹⁵N Glutamine and ¹⁵N Glutamic acid (Cambridge Isotope Laboratory) and was evaluated for incorporation. The incorporation was 0%, 65%, 65%, 0% and 0% respectively according to the Isotope Ratio Calculator (ChemSW) analysis. The peptide 65-71 (Phe-Leu-Tyr-Asp-Gly-Ile-Arg; SEQ ID NO:7) of Sumo was used to compare the labeling efficiency. A non-labeled peptide has a mass of 883.59 Da. It has one glycine and one tyrosine. Therefore, the mass of a labeled peptide should be 884.59 Da. The MS data indicates a mass peak at 884.5 Da for both peptides labeled with either amino acid. The protein synthesis was carried out using the IVPS buffer containing potassium glutamate.

The short dialysis of S30 (a two hour dialysis) gave less than 100% incorporation of ¹⁵N Arginine to CALML3 (80%—Cambridge Isotope Laboratory and 91%—Spectra Stable Isotope). When the prolonged dialysis of S30 (two hour dialysis followed by a change of dialysis buffer and an overnight dialysis) was used to synthesisize the same protein with ¹⁵N Arginine (Spectra Stable Isotope), it gave nearly 100% incorporation. Therefore, the long dialysis was required to get 100% labeling.

### Example 9

### Construction of Vectors for N-Terminal Protein Fusions

Several vectors for expression and purification of proteins from IVPS were made. All the constructs were verified by DNA sequencing. This Example describes the construction of vectors in which the amino terminal side of a cloned protein of interest is fused to one or more desirable fusion protein elements.

The plasmid pEXP1-DEST (SEQ ID:8) (Invitrogen, Carlsbad, Calif.) was used to help create the plasmid pFKI090 (SEQ ID NO:9). The plasmid pEXP 1-DEST comprises two origins of replication (f1 on and pUC ori), an ampicillin resistance gene for positive selection, and a cloning cassette. The cloning cassette contains, in the following order, a T7 promoter operably linked to an RBS (ribosome binding site), a start codon (ATG), a His-tag sequence (6×His), an Xpress™ epitope (Asp-Leu-Tyr-Asp-Asp-Asp-Asp-Lys; SEQ ID:10), an enterokinase (EK) cleavage site, an attR1 site, a chloramphenicol resistance gene (CmR), a ccdB gene, and an attR2 site. The att sites are used to carry out a Gateway™-mediated cloning reaction, in which site-specific recombination results in the removal of the segment of the vector between the attRl and attR2 sites and the replacement of that segment for a gene of interest. The removed segment fragment also contains the ccdB gene, which is useful for negative selection. Because the ccdB gene product kills cells lacking a functional ccdA gene (Bernard et al., J Mol Biol. 226:735, 1992), ccdB+ vectors are propagated in a ccdA⁻ strain, e.g., One Shot® ccdB Survival™ T1 Phage-Resistant Cells [F⁻ mcrA (mrr⁻ hsdRMS mcrBC) 80lacZM15 lacX74 recA1 ara139 Δ(ara-leu)7697 galU galK rpsL (StrR) endA1 nupG tonA::Ptrc ccdA⁻] (Invitrogen). Transformation of cloning reactions into a ccdA⁺ cell ensures that vectors that retain the attR1-attR2 segment will kill their host cells and will thus be excluded from the cloned products due to negative selection.

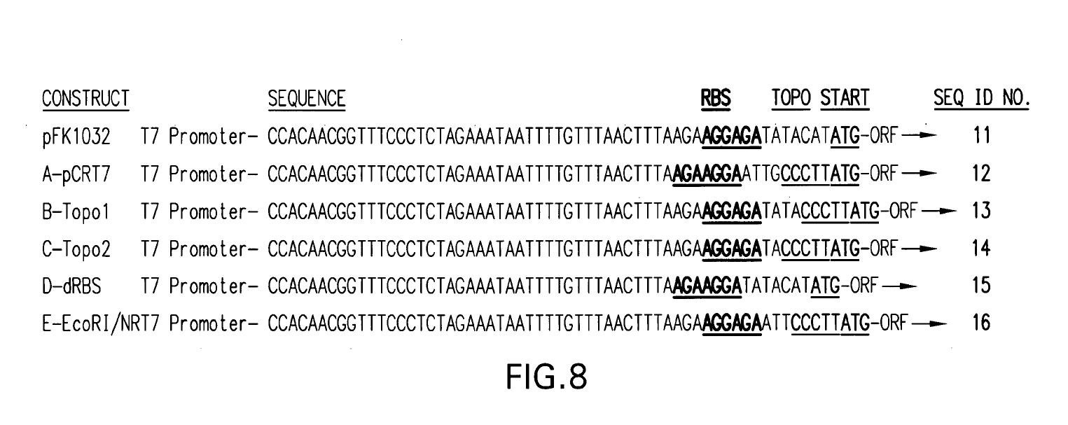

Vector sequences that result in mRNA having sequences that enhance translation in an IVPS reaction that enhance expression of some proteins. In the case of the TOPO® vector described in this Example, expression-enhancing stem-loop structures (Paulus et al., Nucleic Acids Res. 32:e78, 2004) were engineered into the mRNA by adding their corresponding DNA sequences to the vector (FIG. 8).

Five DNA fragments comprising two different ribosome binding sites (RBSs) and different spacings between the RBSs and 5′-CCCTT-3′ TOPO® charging sites were cloned adjacent to the ATG start codon for the cycle3 GFP gene in a pEXP2 derivative. Cell-free synthesis of cycle3 GFP was carried out using pFK1032 and the other constructs in the presence of ³⁵S-Met. Expression lysates were separated on NuPAGE® gel, and proteins were detected by Coomassie staining and autoradiogram. Yield was determined by incorporation of ³⁵S-Methionine, and specific activity determined by Relative Light Units (RLU) per mg of the yield and relative amount of full length protein by densitometry of the full length bands on the autoradiogram. Of the plasmids shown in FIG. 8, the optimal construct was verified in an Expressway reaction to be the one named “TOPO® 2”, and further vectors were generated using this vector.

The plasmid pEXP1-DEST served as a template for the PCR. A PCR fragment was amplified in tandem first using the primers TA-IN-F (SEQ ID NO:17) and TA-B (SEQ ID NO:18) and then the primers OUT-F (SEQ ID NO:19) and TA-B (SEQ ID NO:18) (Table 7). The resulting PCR fragment was digested with XbaI and Bell and cloned into pUCT7GFP, which had been previously digested with the restriction enzymes XbaI and BamHI. Subsequently, two DNA fragments from the resultant plasmid were removed by two cycles of restriction and self-ligation using the restriction enzymes NotI and PstI to yield plasmid pFKI090 (SEQ ID NO:9).

In the studies described herein, 2 types of antibodies to the His tag were employed. Anti-HisG is an antibody that recognizes H-H-H-H-H-H-G (SEQ ID NO:37) (occurring anywhere in the protein) and depends on the glycine for recognition, whereas the anti-His (C-term) antibody recognizes H-H-H-H-H-H-(COOH) (SEQ ID NO:38) at the carboxy terminus and depends on the carboxy group for recognition. Both the anti-HisG and the anti-His(C-term) antibodies, and various labeled derivatives thereof, are commercially available (Invitrogen). Because anti-HisG does not recognize C-terminal His tags, it is used in the experiments described herein to detect N-terminal His tags.

In order to allow detection of cloned proteins of interest comprising N-terminal His tags by anti-HisG, a glycine codon was incorporated adjacent to the 6×His coding sequence in pFKI090, a 100-bp Xbal I-Bsu361 fragment in pFKI090 was substituted by a DNA fragment that was amplified from pFKI090 using primers pEXP1-TOPO®-FOR (SEQ ID NO:20) and pEXP1-TOPO®-REV (SEQ ID NO:21). The PCR product was digested with the Xbal I and Bsu361 restriction enzymes and ligated into pFKI090 backbone DNA, also digested with Xbal I and Bsu361, thus generating the plasmid pEXP5-NT/TOPO® (SEQ ID NO:39).

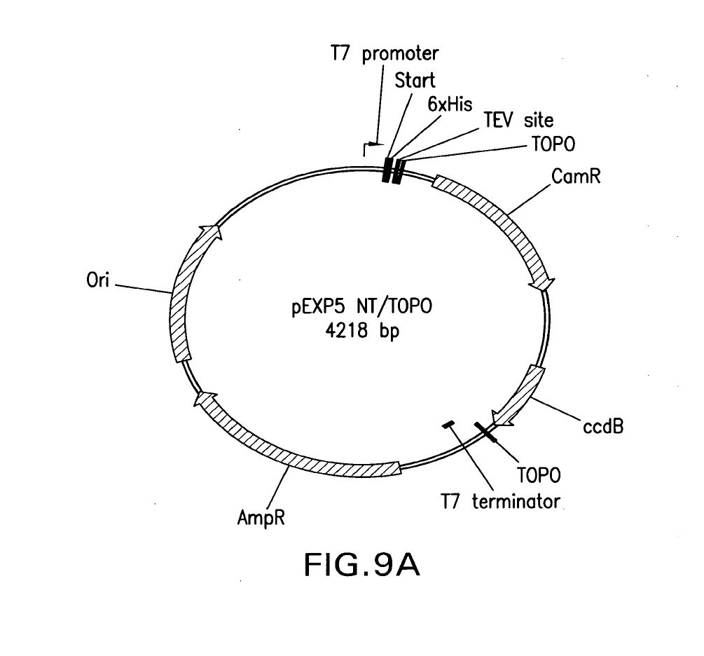

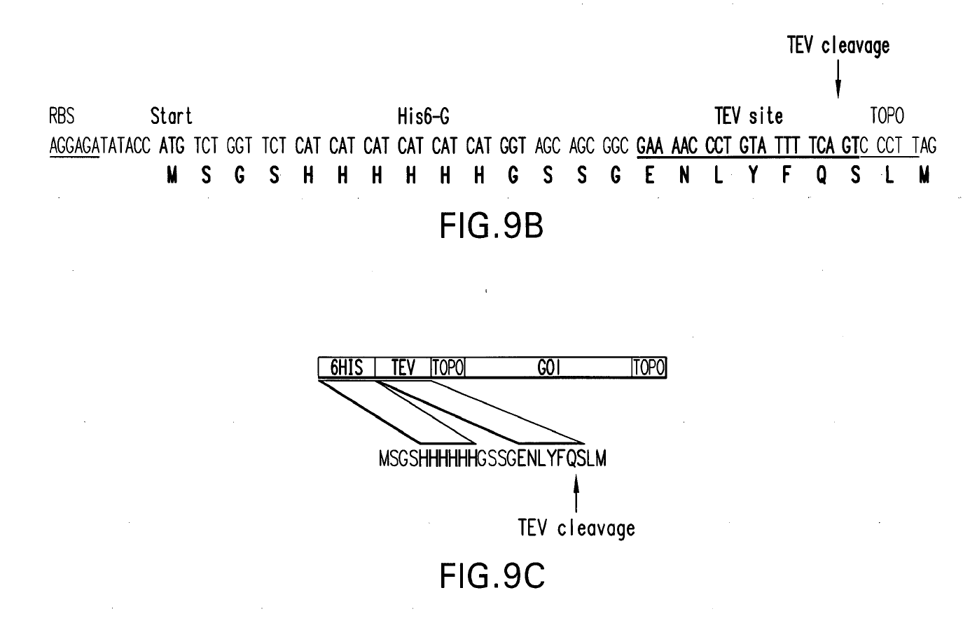

FIG. 8 illustrates the coding elements in the pEXP5-NT/TOPO® vector, which is shown in FIG. 9. Effective TOPO®-TA cloning will eliminate the ccdB gene, allowing for negative selection, as is described above. This construct adds only 21 amino acids onto the N-terminus of the gene of interest and leaves only 2 additional amino acids on the synthesized product after protease (TEV) cleavage (FIGS. 9B and 9C). This compares favorably with the pEXP1-DEST vector, which adds an extra 51 amino acids to the protein of interest as expressed from the vector and, even after protease (EK) cleavage, leaves 19 additional amino acids in the final protein product.

TABLE 7

OLIGONUCLEOTIDES USED FOR CONSTRUCTION OF
THE N- AND C-TERMINAL VECTORS

| | | |
|---|---|---|
| Oligonucleotide | | SEQ ID |
| Name | Sequence (5′ to 3′) | NO.: |

| | | |
|---|---|---|
| TA-IN-F | AGCAGCGGCGAAAACCTGTATTTTCAGTCCCTTAGGATGCAG | 17 |
| | GTACGGAGCGGCCGCTGAACCTGCTACATGCCGCGGCCGC | |
| | ATTAGGCAC | |
| TA-B | AGCGTCGAGGTGATCACCCTTAGAGTGCAGGTGCCTGCTGC | 18 |
| | AGTCCTTCTGCACCTGCAGACCGATTGTGTATAAGGGAGCC | |
| | TGAC | |
| OUT-F | GAGGTCTAGAAATAATTTTGTTTAACTTTAAGAAGGAGATATA | 19 |
| | CCATGTCTGGTTCTCATCATCATCATCATCATAGCAGCGGCG | |
| | AAAACCT | |
| PEXP1-TOPO ®- | GGTTTCCCTCTAGAAATAATTTTGTTTAAC | 20 |
| FOR | | |
| PEXP1-TOPO ®- | CCTAAGGGACTGAAAATACAGGTTTTCGCCGCTGCTACCAT | 21 |
| REV | GATGATGATG | |
| IDT-TOPO ®-B- | CCCTTATTCCGATAGTG | 22 |
| TA-F* | | |
| IDT-TOPO ®-B- | CACTATCGGAATA | 23 |
| TA-R* | | |
| NMRFor | CCCGAAGATCTCGATCCCGCGAAATTAATACG | 24 |
| RIRev | CTCCTTTGCTAGCCATAAGGGAATTCTCCTTCTTAAAG | 25 |
| AmpBSAFor | GGAGCCGGTGAGCGTGGGTCACGCGGTATCATT | 26 |
| AmpBSARev | GACCCACGCTCACCGGCTCCAGATTTATCAG | 27 |
| 45BSAFor | CGACTCACTATAGGGAGACACACAACGGTTTCC | 28 |
| 45BSARev | GTCTCCCTATAGTGAGTCGTATTAATTTCGC | 29 |
| Rev AatII | AGTGCCACCTGACGTCTAAG | 30 |
| BSATAfor | GCGGAATTCGGTCTCAAGGGTCATCATCACCATCACCATTG | 31 |
| BSATA | CCGCGAATTCGGTCTCAAGGGTATCTCCTTC | 32 |
| TA ECORI | ATACCCTTGAGACCGAATTCGGCAATTCGCGCAATTGCGG | 33 |
| CalFOR | ATGGCCGACCAGCTGACTGAGGAGC | 34 |
| CalREV | CTTGGACACCAGCACACGGACAAAC | 35 |

*These oligonucleotides were ordered 5′-phosphorylated and HPLC purified from Integrated DNA Technologies.

### Example 10

### Topo® Cloning Using Pexp-NtlTopo®

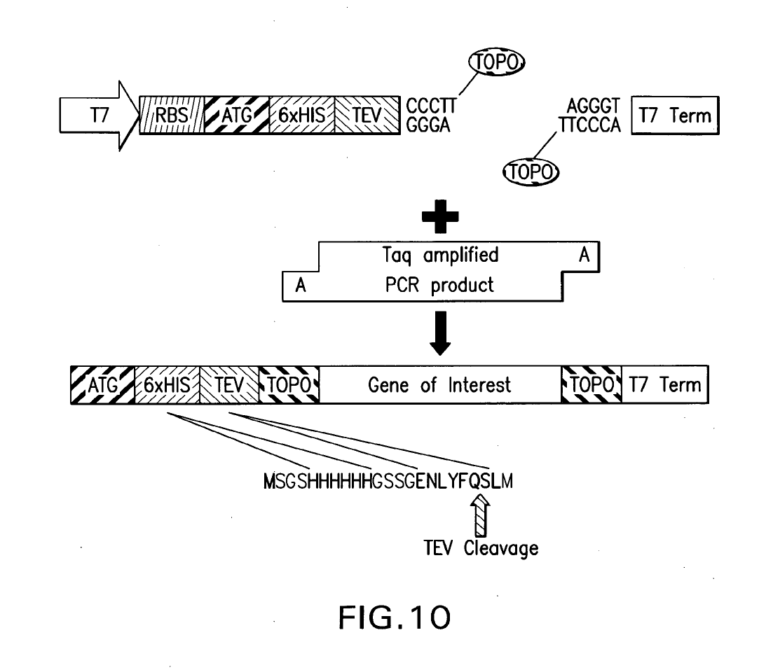

A general scheme for TOPO® cloning using the pEXP5-NT/TOPO® vector is shown in FIG. 10. Some examples of TOPO® cloning using the pEXP5-NT/TOPO® vector are as follows.

### TOPO® Charging

The plasmid pEXP5-NT/TOPO® (100 μg) was digested to completion with 100 U of the restriction enzyme BfuA1 (NEB) in a final volume of 500 μl at 50° C. for 16 h. The DNA was ethanol-precipitated by the addition of 80 μl of 4M LiCl and 1 ml of ethanol. The mix was incubated for 10 min at −80° C. and centrifuged for 30 min at 4° C. The pellet was washed with 70% ethanol and resuspended in 100 μl of sterile water.

Five micrograms of the digested DNA, equivalent to 1.8 μmoles of each of the fragments, were used for TOPO®-adaptation as follows. First, the digested DNA (SEQ ID NO:40) was ligated to a phosphorylated and HPLC-purified adaptor oligonucleotide. The oligonucleotide was first incubated at 100° C. for 2 min. The ligation reaction contained 5 μg of digested plasmid, 3.2 nmoles (approximately 17 μg) of the phosphorylated and HPLC-purified oligonucleotide IDT-TOPO®-B-TA-F, 800 U of T4 DNA ligase (NEB), 5 μl of 10× ligase buffer (NEB) and sterile water to The reaction proceeded for 30 min at room temperature. Then, free oligonucleotides were removed using the PureLink PCR Purification Kit (Invitrogen) essentially according to the manufacturer's instructions. The DNA was eluted with 50 μl of sterile water, and charged with Vaccinia TOPO®isomerase I. The following reagents were added in the given order in a total reaction volume of 50 μl (Table 8).

TABLE 8
TOPO ®-CHARGING REACTION

| Reagent | Amount | |
|---|---|---|
| DNA | 2 | μg |
| IDT-TOPO ®-B-TA-R | 200 | pmoles (approximately 0.8 μg) |
| oligonucleotide | | |
| Vaccinia TOPO ® isomerase I | 2 | μg |
| NEB 10x buffer #1 | 5 | μl |
| Water | to 50 | μl |

The TOPO®isomerase reaction was incubated for 15 min at 37° C. Then, 6 μl of 10× Stop TOPO® buffer was added, and the reaction was incubated for 5 min at RT. The TOPO®-charged DNA was gel-purified and stored as directed for other TOPO® vectors by the manufacturer (Invitrogen).

### TOPO® Cloning

In order to assess the TOPO® TA cloning performance of the vector, a DNA fragment that encodes the lacZ alpha peptide as a reporter was used. Successful cloning of this fragment into the vector results in blue colonies of TOP10-transformed cells on an X-gal plate, whereas other structures will yield white colonies. A single TOPO® reaction using the charged DNA produced 2,712 colonies, of which only 44 (1.6%) where white. Nearly all (98.4%) colonies were blue, i.e., they expressed lacZ, indicating highly efficient and accurate cloning.

TOPO®-TA cloning of PCR fragments was performed as directed by the TOPO® TA cloning manual (Invitrogen). Human ORFs were amplified using Platinum® PCR SuperMix High Fidelity (Invitrogen) and gene specific primers. For ‘No stop’ C-terminal constructs, the Reverse primer did not include the TAG sequence. Briefly, 1 μA of the PCR reaction was incubated with 1 μl of the TOPO®-vector and 1 μl of the salt solution in 6 μl final reaction volume for 10 min at RT. Resulting constructs were transformed into Top10 cells and screened by colony PCR (Zon et al., Biotechniques 7:696, 1989). Positive clones were then verified by sequencing.

### Example 11

### Construction of Vectors for C-Terminal Protein Fusions

Several vectors for expression and purification of proteins from IVPS were made. All the constructs were verified by DNA sequencing. This Example describes the construction of a vector in which the carboxy-terminal side of a cloned protein of interest is fused to one or more desirable fusion protein elements. The vectors give the researcher the option to generate a full-length native protein of interest by cloning that includes a stop codon or, when cloned with no stop codon included, the C-terminal His-tag will be expressed as part of a fusion protein comprising the protein of interest and 8 additional amino acids (KGHHHHHH; SEQ ID NO:41).

The N-terminal sequence of the vector between the ribosomal binding site and the start codon was analyzed to determine the best spacing for the TOPO® site. The plasmid pFKI032 (SEQ ID NO:42) was used as the template for construction of the test constructs and served as the positive control vector. The pFKI032 plasmid carries the native T7 sequences from the T7 promoter to the first ATG of the cycle3 GFP gene with a stop codon. The 3′ sequences after the stop codon include an atttL2 site and a T7 terminator.

DNA sequences containing the RBS and TOPO® site variants were cloned by PCR mutagenesis as described. The TOPO® 2 version (SEQ ID NO:14) was used as the starting material for the construction of the pEXP5-CT/TOPO® vector (SEQ ID NO:43). In brief, this was done by removing two existing BsaI sites; adding 5′ and 3′ BsaI sites, TOPO® cloning sites and a 6×His sequence; and cloning a larger stuffer fragment between the two BsaI sites in order to reduce background.

The pEXP5-CT/TOPO® vector was constructed by PCR of the pFKI032 (SEQ ID NO:42) plasmid with primers NMR For (SEQ ID NO:24) and RIRev (SEQ ID NO:25) (Table 7). The PCR fragment contained the RBS and TOPO® 2 site variant. A 120 by fragment that was gel purified after digestion with BglII and NheI. The pFK1032 vector was also digested with BglII and NheI. The pFK1032 backbone was purified and used in a ligation with the 120 by BglII and NheI fragment carrying the new RBS and an EcoRI site. A positive clone was sequence verified and used as the template for the mutagenesis reactions.

In order to remove undesirable BsaI sites from the vector, the two existing BsaI sites were mutated using the Gene Tailor Kit (Invitrogen). The AmpBSAFor primer and AmpBSARev primer (Table 7) were used to substitute the Ser13 TCT codon for Ser TCA in the ampicillin resistance gene. The 45BSAFor primer (SEQ ID NO:28) and the 45BSARev primers (SEQ ID NO:29) were used to insert a single adenine (A) nucleotide at position 45 of the untranslated sequence. After verification of the two mutations by sequencing, a positive clone was identified.

The desired 3′ BsaI site, TOPO® adaption site and 6×His encoding sequences were added to the newly created vector by PCR of pCRT7-CT/TOPO® (Invitrogen, SEQ ID NO:44) with the primers Rev AatII (SEQ ID NO:30) and BSATAfor (SEQ ID NO:32). The PCR product and mutated vector were digested with EcoRI and AatII. The 240 by fragment was purifed and ligated into the prepared backbone. This step also removed the attB2 site. A positive clone was sequenced and was used in subsequent constructions.

To add the desired 5′ BsaI site and TOPO® adaption sites, the BSATA (SEQ ID NO:32) and NMRFor (SEQ ID NO:24) primers were used to amplify a PCR fragment containing the final 5′ sequence of the pEXP5-CT/TOPO® vectors, including the RBS, TOPO® adaption sites, BsaI site and EcoRI site. The 126 by PCR product was purified after digestion with BglII and EcoRI, and ligated into the prepared backbone digested with the same enzymes. A positive clone, pEXP5-CT/TOPO®-SM (SEQ ID NO:45), was isolated. This clone had two BsaI sites separated by an 18 base stuffer fragment containing an EcoRI cut-back site. A larger (27 bp) stuffer was added between the BsaI sites. The pEXP5-CT/TOPO®-penultimate vector was used as the template in a PCR reaction with the primers NMRfor (SEQ ID NO:24) and TA ECORI (SEQ ID NO:33). The PCR product was digested with BglII and MfeI and ligated into pEXP5-CT/TOPO®-SM DNA digested with BglII and EcoRI. After PCR colony screening, a clone was selected and sequenced in its entirety.

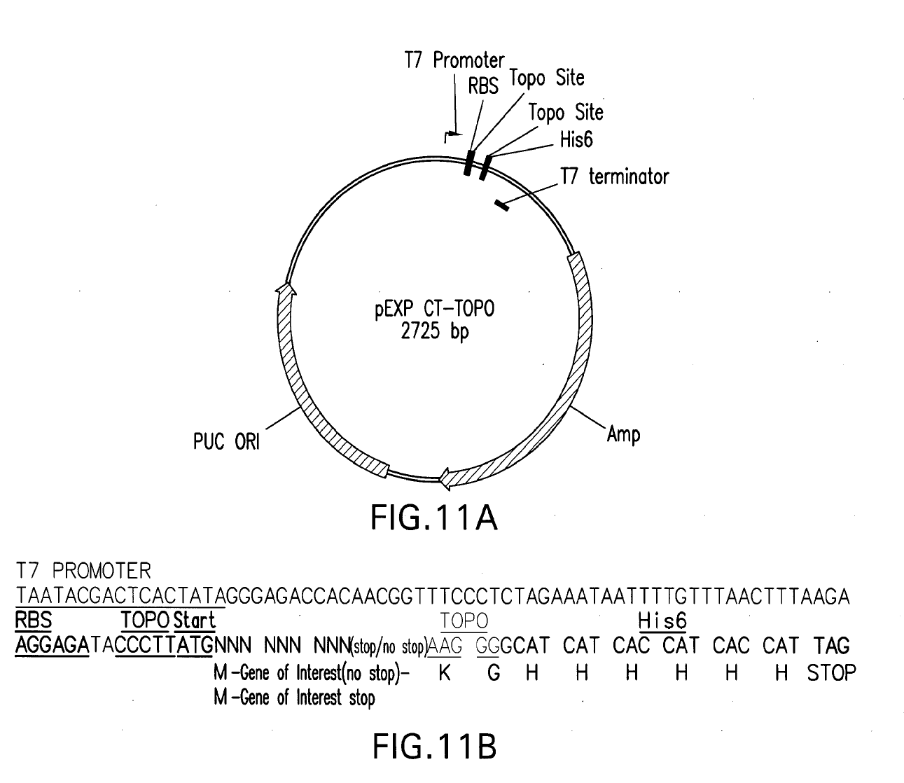

This plasmid pEXP5-CT/TOPO® (SEQ ID NO:42) is illustrated in FIG. 11. The gene of interest may be inserted with a stop. If no stop codon included, the C-terminal His-tag will be expressed adding 8 additional amino acids to the carboxy terminus of the cloned protein of interest.

### Example 12

### Topo® Cloning Using Pexp5-Ct/Topo®

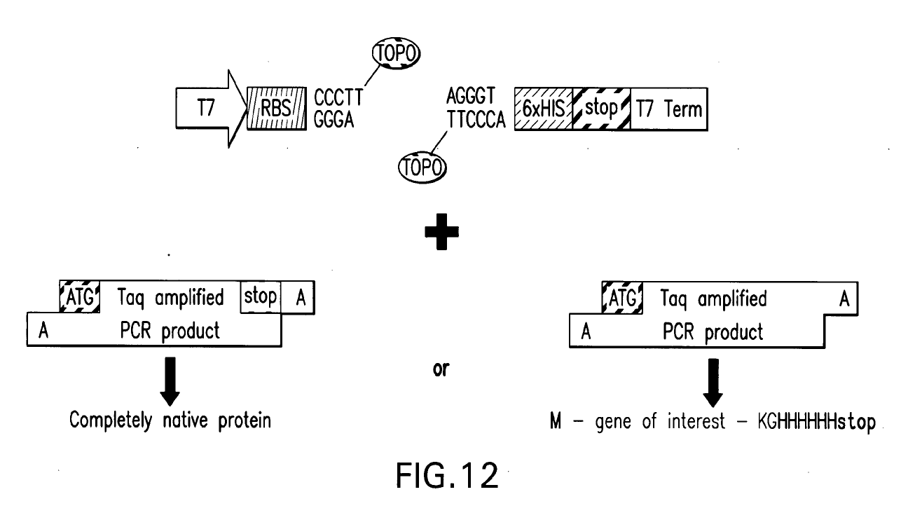

A general scheme for TOPO® cloning using the pEXP5-CT/TOPO® vector is shown in FIG. 12. Some examples of TOPO® cloning using the pEXP5-CT/TOPO® vector are as follows.

### TOPO® Charging

The plasmid pEXP5-CT/TOPO® (100 μg) was linearized with EcoRI (NEB) by digestion with 500 U in a volume of 400 μl for 2 hours in NEB Buffer 3. The vector was then digested with 500 U of BsaI (NEB) by supplementing the reaction with the restriction enzyme and incubating at 50° C. for 4 h. The DNA was ethanol-precipitated by the addition of 40 μl 3M sodium acetate and 880 μl of ethanol. The mix was incubated for 10 min at −80° C. and centrifuged for 30 min at 4° C. The pellet was washed with 70% ethanol and resuspended in 68 μl of TE. The stuffer fragment was removed by isopropanol precipitation, which was performed by adding 6 μl 3M sodium acetate and 73 μl isopropanol and incubating 5 minutes at RT before centrifuging for 5 minutes. The pellet was washed with 70% ethanol and resuspended in 100 μl sterile water.

Ten micrograms of the digested DNA (SEQ ID NO:47) were used for TOPO®-adaptation. First, the prepared DNA was ligated to a phosphorylated and HPLC-purified adaptor oligonucleotide overnight; the oligonucleotide was first incubated at 100° C. for 2 min. The ligation reaction contained 10 μg of digested plasmid, 25 μg (200 molar excess) of the phosphorylated and HPLC-purified oligonucleotide IDT-TOPO®-B-TA-F (SEQ ID NO:22), 400 U of T4 DNA ligase (NEB), 10 μl of 10× ligase buffer (NEB) and sterile water to 100 μl. The following day, the free oligonucleotides were removed using the PureLink PCR Purification Kit (Invitrogen) essentially according to the manufacturer's instructions. The DNA was eluted with 50 μl of sterile water. Finally the DNA was charged with Vaccinia TOPO®isomerase I. The following reagents were added in the exact order in a total reaction volume of 100 μl (Table 9). The reaction was incubated at 15 minutes at 37° C. Then, 11 μl of 10× Stop TOPO® buffer was added and the reaction was incubated for 5 minutes at RT. The TOPO®-charged DNA was gel-purified and diluted to a final estimated concentration of 2.5 μA per μg (1250 μl total), and was stored as directed for other Invitrogen TOPO® vectors.

TABLE 9
Topo ®-Charging Reaction

| Reagent | Amount | |
|---|---|---|
| DNA | 3 | μg |
| IDT-TOPO ®-B-TA-R oligonucleotide | 5 | μg |
| Vaccinia TOPO ® isomerase I | 10 | μl |
| xcvNEB 10x buffer #1 | 10 | μl |
| Water | to 100 | μl |

### TOPO® Cloning

The pEXP5-CT/TOPO® construct was compared to the Gateway® pEXP4 vector for expression levels of full-length protein essentially as described above. Expression was determined by Phosphorimager anaylsis of the full-length product from each lane divided by the total number of methionines in each expression construct. Numbers were normalized to the highest expresser for each pair of ORFs and presented as a percentage.

Kinase clones IOH6416 (1826), IOH5211 (4914), IOH6368 (4553) all contain stop codons (because the ORF Entry clones used for the Gateway recombination all contain stop codons) were compared for expression levels in the two vectors. In most cases, overall expression levels were similar, except for IOH6416 (1826), which generated almost 5 times higher yield in the pEXP5-CT/TOPO® vector. In addition, there was less background with the pEXP-CT products as compared to the pEXP4 products.

### Example 13

### Protein Expression From Topo® Vectors

Comparison of Expression Levels from pEXP1-DEST versus pEXP5-NT/TOPO®

In order to assess the relative quality and yield of products expressed from the TOPO® vectors, 6 different mammalian ORFs were (1) TOPO®-cloned into the pEXP5-NT/TOPO® vectors and (2) cloned by attL x attR recombination into the pEXP1-DEST vector using Gateway™ technology. Cell-free reactions were performed with the “feed” method as described above. Two microliter samples were acetone-precipitated and loaded on an SDS-PAGE gel. After electrophoresis, the gel was stained with Coomassie blue and exposed to a phosphorimager screen. The relative abundance of the full-length products was performed by phosphor-storage autoradiography, and analyzed on a Typhoon 8600 Variable-mode Imager using the IMAGEQUANT software (Amersham Pharmacia Biotech).

Expression levels and amounts of full-length product from the N-terminal constructs were compared. Expression levels were determined by PhosphorImager anaylsis of the full-length product from each lane divided by the total number of methionines in each expression construct. Numbers were normalized to the highest expresser for each pair of ORFs and presented as a percentage.

The results (show that out of 6 sequences tested, 4 of them expressed on average two-fold higher from the TOPO® vector than when expressed from pEXP 1. Only one ORF (IOH11046) exhibited higher yields when expressed from pEXP1-DEST and another one (IOH3588) expressed at comparable levels. Other proteins such as GFP expressed at significant higher levels from the TOPO® vector (not shown). In addition, virtually all the sequences expressed from the TOPO® vector produced less truncated or incomplete products when compared to those from pEXP1.

Comparison of Expression from pEXP5-CT/TOPO® and pEXP5-NT/TOPO® Vectors

The vectors pEXP5-CT/TOPO® (CT) and pEXP5-NT/TOPO® (NT) were used to express the CALML3, IOH6416, IOH5211 and IOH6368 proteins. The proteins synthesized in in vitro synthesis reactions using these ORF cloned in the expression plasmids were electrophoresis on a 4-12% NuPAGE® Bis/Tris gel, and the gel was subjected to autoradiography to analyze protein levels. FIG. 13 provides a graph comparing the expression level of the four ORFs from either pEXP5-CT/TOPO® or pEXP5-NT/TOPO®.

While testing expression levels of various ORFs cloned into the new TOPO® vectors, it was observed that some genes expressed better in the N-terminal vector while others performed better with the C-terminal. Protein expression levels are known to be protein dependent, and simply moving a coding element like the 6×His tag from one end to the other may have a dramatic effect on protein yields In all cases, strong differences in expression levels are observed with the movement of the tag, with the exception of CALML3. In this case, the CALML3 ORF was cloned into the CT-vector with a stop codon, so the expression levels are comparing a fusion protein with an N-terminal tag to a protein with no 6×His-tag.

### Example 14

### Protein Detection And Purification

Amino-Terminal Fusion Protein (pEXP5-NT/TOPO®)

Detection and purification of proteins via the His6 tag and nickel resin was verified for proteins expressed in vitro from the TOPO® vectors. For the N-terminal vector, the synthesized product was treated with TEV protease to remove the His6 tag, and removal was verified by failure to bind to fresh Ni-NTA resin.

For purification of 6×His-tagged proteins expressed from the pEXP5-NT/TOPO® vector, a 100 μl Expressway™ Milligram reaction containing synthesized GFP was loaded directly onto Ni-NTA resin. Two (2) μl samples of the loaded material, flow-through, 3 washes (W) and 3 elutions (E) were analyzed. The samples were electrophoresed through a 4-12% NuPAGE® gel, which was stained with Coomassie. The results show that most of the proteins in the sample were not bound to the resin and were thus present in the flow-through. However, the His-tagged protein was retained and remained bound during 3 washes, and was released during a first elution. Subsequent elutions contained very little (if any) protein, indicating that the His-tagged protein was efficiently released by a single elution.

Protein products prepared from the pEXP5-NT/TOPO® vector were also efficiently cleaved by the TEV protease. Samples from an IFPS reaction of a pEXP5-NT/TOPO® construct comprising GFP were loaded into the column, and samples were taken of the initial flow-through, 2 washes with 5 mM Imidazole, and 2 washes with 20 mM Imidazole. The protein was eluted with 200 mM Imidazole. The eluted protein was digested with the TEV protease and efficient proteolysis was seen. The TEV-treated protein was not retained by a second ProBond™ column, indicating removal of the His6 tag as expected.

Carboxy-Terminal Fusion Protein (pEXP5-CT/TOPO®)

Plasmid pEXP5-CT/CALML3 (no stop codon) was expressed in a 200 μl Expressway-Milligram reaction. Twenty-five (25) μl of the reaction was loaded directly onto a Ni-NTA column. The column was washed 3 times and the bound protein eluted in 4 fractions. Samples of each fraction were separated on two 4-12% NuPAGE® gels. One gel was stained with SimplyBlue™ (Invitrogen), and the other was transferred to nitrocellulose and probed with anti-HisC using the Western Breeze™ anti-mouse Chemiluminescent Kit (Invitrogen). In order to detect His-tagged proteins produced from pEXP5-CT/TOPO® constructs, an anti-HisC (C-term) antibody (Invitrogen) was used. The Anti-His(C-term) antibody (Lindner et al., BioTechniques 22:140, 1997) is a monoclonal antibody that recognizes a polyhistidine amino acid sequence at the carboxy-terminus of proteins. The anti-His(C-term) antibody recognizes the sequence -His-His-His-His-His-His-COOH, and the free carboxy-terminus of the terminal histidine residue is an element of the epitope recognition site. The results showed that the CALML3-6× His protein was efficiently purified on the Ni-NTA column.

### Example 15

### Expressway™ Milligram Ivtt Kits

Expressway™ IVPS systems (Invitrogen, Carlsbad, Calif.) include kits for expressing milligram amounts of proteins. Such kits include: 1) an IVPS E coli extract, 2) 2.5× IVPS Reaction Buffer, 3) 2× IVPS Feed Buffer, 4) T7 Enzyme Mix, 5) 50 mM amino acids mix (minus met and cys), 6) 75 mM Met, and 7) 75 mM Cys. The kit also includes nuclease-free distilled water. The kit also includes pEXP5-NT/CALML3 expression control plasmid.

The E. coli extract provided in the kit is made by resuspending cell used to make the extract in a buffer that includes Triton X-100 at a final concentration of 0.1% prior to lysing the cells.

2.5× IVPS reaction buffer is: 145 mM HEPES-KOH, pH 7.6, 4.25 mM DTT, 3.0 mM ATP, 2.2 mM UTP, 2.2 mM CTP, 2.2 mM GTP, 85 micrograms per milliliter folinic acid, 75 mM acetyl phosphate, 575 mM potassium acetate, 30 mM magnesium acetate, 200 mM NH4OAc, 1.625 mM cAMP, 75 mM PEP, 5% PEG.

2× Feed Buffer is: 115 mM HEPES-KOH, pH 8, 3.4 mM DTT, 68 micrograms per milliliter folinic acid, 460 mM potassium acetate, 28 mM magnesium acetate, 160 mM NH4OAc, 4 mM CaCl2, 1.3 mM cAMP, 90 mM glucose-6-phosphate, and 1 mM NAD.

Some kits also contain cloning vectors pEXP5-NT/TOPO® and pEXP5-CT/TOPO®. Some kits also include competent cells.

The kits include enough reagents for multiple IVPS reactions.

The kits include instructions for use.

### Example 16

### Expressway™ Nmr Ivtt Kits

Expressway™ IVPS systems (Invitrogen, Carlsbad, Calif.) include kits for expressing proteins that can be labeled during IVPS for NMR analysis. Such kits include: 1) an IVPS E. coli extract, 2) 2.5× IVPS Reaction Buffer, 3) 2× IVPS Feed Buffer, 4) T7 Enzyme Mix, 5) 200 mM solutions of each amino acid except Leu, provided separately and 6) 150 mM leu,. The kit also includes nuclease-free distilled water. The kit also includes pEXP5-NT/CALML3 expression control plasmid.

The E. coli extract provided in the kit is made by resuspending cell used to make the extract in a buffer that includes Triton X-100 at a final concentration of 0.1% prior to lysing the cells.

2.5× IVPS reaction buffer is: 145 mM HEPES-KOH, pH 7.6, 4.25 mM DTT, 3.0 mM ATP, 2.2 mM UTP, 2.2 mM CTP, 2.2 mM GTP, 85 micrograms per milliliter folinic acid, 75 mM acetyl phosphate, 575 mM potassium acetate, 30 mM magnesium acetate, 200 mM NH4OAc, 1.625 mM cAMP, 75 mM PEP, 5% PEG.

2× Feed Buffer is: 115 mM HEPES-KOH, pH 8, 3.4 mM DTT, 68 micrograms per milliliter folinic acid, 460 mM potassium acetate, 28 mM magnesium acetate, 160 mM NH4OAc, 4 mM CaCl2, 1.3 mM cAMP, 90 mM glucose-6-phosphate, and 1 mM NAD.

Some kits also contain cloning vectors pEXP5-NT/TOPO® and pEXP5-CT/TOPO®.

The kits include enough reagents for multiple IVPS reactions.

The kits include instructions for use.

Unless otherwise defined, all technical and scientific terms used herein have the meaning commonly understood by one skilled in the biotechnology art. All publications, patent applications, patents, and other references mentioned herein are incorporated by reference in their entirety. In case of conflict, the present specification, including definitions, will control.

Headings are for the convenience of the reader, and are not intended to limit the invention.

All references cited herein are incorporated by reference in their entireties.

The details of one or more embodiments of the invention are set forth in the accompanying drawings and the description below. Other features, objects, and advantages of the invention will be apparent from the description.

1. A system for in vitro synthesis of proteins, comprising a cell extract that comprises a detergent or a surfactant, wherein the cell extract is made by lysing cells to obtain a cell lysate and separating a supernatant fraction from the cell lysate, wherein one or more detergents or surfactants is added to the cells prior to lysis or the cell lysate prior to separating a supernatant fraction.

2. The in vitro synthesis system of claim 1, wherein the cell extract is made by adding one or more detergents or surfactants to the cell lysate prior to separating a supernatant fraction.

3. The in vitro synthesis system of claim 2, wherein said separating IS centrifuging said cell lysate and removing a supernatant as a cell extract.

4. The in vitro synthesis system of claim 1, wherein the cell extract is made by adding one or more detergents or surfactants to wherein one or more detergents or surfactants is added to the cells prior to lysis.

5. The in vitro system of claim 1, wherein said at least one detergent or surfactant is at least one detergent.

6. The in vitro system of claim 5, wherein said at least one detergent is a nonionic detergent or a zwitterionic detergent.

7-26. (canceled)

27. A method of synthesizing a protein, comprising:
adding to a cell extract: amino acids, at least one energy source, and a nucleic acid template, to make an in vitro protein synthesis mixture;
wherein the cell extract is made from cells or a cell lysate that has been treated with at least one surfactant or detergent prior to making the extract; and
incubating the vitro protein synthesis mixture to synthesize the protein.

28. The in vitro synthesis system of claim 27, wherein said cell extract is made from cells that have been treated with said at least one surfactant or detergent prior to centrifuging the extract to isolate a cell lysate supernatant.

29. The method of claim 28, wherein is the cell extract is made from treating the cells with a detergent.

30. The method of claim 29, wherein the detergent is a zwitterionic or nonionic detergent.

31. The method of claim 30, wherein said at least one detergent is a nonionic detergent.

32. The method of claim 30, wherein said detergent is a detergent of the Brij® series, a detergent of the Zwittergent series, a detergent of the Triton series, or a glycopyranoside detergent.

33-45. (canceled)

46. The method of claim 27, further comprising:
After incubating the reaction mixture for a period of time, adding to the synthesis mixture a feeding solution that comprises a buffer, amino acids, at least one additional energy source, wherein the at least one additional energy source is different from the at least one energy source of the initial synthesis mixture to make an extended synthesis mixture; and
Incubating the extended synthesis mixture for an additional period of time to synthesis at least one protein.

47. A method of synthesizing a protein, comprising:
Adding to a cell extract amino acids, at least one energy source, and a nucleic acid template to make an initial in vitro protein synthesis mixture;
Adding to the initial synthesis mixture a feeding solution that comprises a buffer, amino acids, and at least one additional energy source, wherein the at least one additional energy source is different from the at least one energy source of the initial synthesis mixture to make an extended synthesis mixture; and
Incubating the extended synthesis mixture for a period of time to synthesize the protein.

48. The method of claim 47, wherein said at least one additional energy source is different from the energy sources provided in the initial synthesis mixture.

49. The method of claim 48, wherein said at least one additional energy source is not an enzyme.

50. The method of claim 49, wherein said at least one additional energy source is a glycolytic intermediate.

51. The method of claim 49, wherein said at least one additional energy source is fructose-6-phosphate, glucose-6-phosphate, or 3 phosphoglycerate.

52. The method of claim 47, wherein said feeding solution further comprises a cofactor.

53-67. (canceled)


SEQUENCE LISTING (51 sequences)

SEQ ID NO: 1 - protein, 109 aa, Artificial Sequence
  Description of Artificial Sequence Synthetic polypeptide
MSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGPGGGSHHHHHH

SEQ ID NO: 2 - protein, 5 aa, Artificial Sequence
  Description of Artificial Sequence Synthetic peptide
RLMEA

SEQ ID NO: 3 - protein, 38 aa, Artificial Sequence
  Description of Artificial Sequence Synthetic polypeptide
IQADQTPEDLDMEDNDIIEAHREQIGGPGGGSHHHHHH

SEQ ID NO: 4 - protein, 8 aa, Artificial Sequence
  Description of Artificial Sequence Synthetic peptide
GCITTREL

SEQ ID NO: 5 - protein, 8 aa, Artificial Sequence
  Description of Artificial Sequence Synthetic peptide
CITTRELG

SEQ ID NO: 6 - protein, 9 aa, Artificial Sequence
  Description of Artificial Sequence Synthetic peptide
TVMRSLGQN

SEQ ID NO: 7 - protein, 7 aa, Artificial Sequence
  Description of Artificial Sequence Synthetic peptide
FLYDGIR

SEQ ID NO: 8 - DNA, 4622 bp, Artificial Sequence
  Description of Artificial Sequence Synthetic polynucleotide
gatctcgatcccgcgaaattaatacgactcactatagggagaccacaacggtttccctctagaaataattttgtttaactttaagaaggagatatacatatgcggggttctcatcatcatcatcatcatggtatggctagcatgactggtggacagcaaatgggtcgggatctgtacgacgatgacgataaggatcatcaaacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatctagaggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttcgatcgaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagagtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagcgctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgca

SEQ ID NO: 9 - DNA, 4218 bp, Artificial Sequence
  Description of Artificial Sequence Synthetic polynucleotide
taatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacaatcggtctgcagcagcacctgctctaagggtgatccggctgctaacaaagcccgaaaggaagctgagttggctgctgccaccgctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatctcgatcccgcgaaattaatacgactcactatagggagaccacaacggtttccctctagaaataattttgtttaactttaagaaggagatataccatgtctggttctcatcatcatcatcatcatagcagcggcgaaaacctgtattttcagtcccttagggcaggtacggagcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgct

SEQ ID NO: 10 - protein, 8 aa, Artificial Sequence
  Description of Artificial Sequence Synthetic peptide
DLYDDDDK

SEQ ID NO: 11 - DNA, 60 bp, Artificial Sequence
  Description of Artificial Sequence Synthetic oligonucleotide
ccacaacggtttccctctagaaataattttgtttaactttaagaaggagatatacatatg

SEQ ID NO: 12 - DNA, 60 bp, Artificial Sequence
  Description of Artificial Sequence Synthetic oligonucleotide
ccacaacggtttccctctagaaataattttgtttaactttaagaaggaattgcccttatg

SEQ ID NO: 13 - DNA, 62 bp, Artificial Sequence
  Description of Artificial Sequence Synthetic oligonucleotide
ccacaacggtttccctctagaaataattttgtttaactttaagaaggagatatacccttatg

SEQ ID NO: 14 - DNA, 60 bp, Artificial Sequence
  Description of Artificial Sequence Synthetic oligonucleotide
ccacaacggtttccctctagaaataattttgtttaactttaagaaggagatacccttatg

SEQ ID NO: 15 - DNA, 58 bp, Artificial Sequence
  Description of Artificial Sequence Synthetic oligonucleotide
ccacaacggtttccctctagaaataattttgtttaactttaagaaggatatacatatg

SEQ ID NO: 16 - DNA, 61 bp, Artificial Sequence
  Description of Artificial Sequence Synthetic oligonucleotide
ccacaacggtttccctctagaaataattttgtttaactttaagaaggagaattcccttatg

SEQ ID NO: 17 - DNA, 91 bp, Artificial Sequence
  Description of Artificial Sequence Synthetic oligonucleotide
agcagcggcgaaaacctgtattttcagtcccttaggatgcaggtacggagcggccgctgaacctgctacatgccgcggccgcattaggcac

SEQ ID NO: 18 - DNA, 86 bp, Artificial Sequence
  Description of Artificial Sequence Synthetic oligonucleotide
agcgtcgaggtgatcacccttagagtgcaggtgcctgctgcagtccttctgcacctgcagaccgattgtgtataagggagcctgac

SEQ ID NO: 19 - DNA, 92 bp, Artificial Sequence
  Description of Artificial Sequence Synthetic oligonucleotide
gaggtctagaaataattttgtttaactttaagaaggagatataccatgtctggttctcatcatcatcatcatcatagcagcggcgaaaacct

SEQ ID NO: 20 - DNA, 30 bp, Artificial Sequence
  Description of Artificial Sequence Synthetic oligonucleotide
ggtttccctctagaaataattttgtttaac

SEQ ID NO: 21 - DNA, 51 bp, Artificial Sequence
  Description of Artificial Sequence Synthetic oligonucleotide
cctaagggactgaaaatacaggttttcgccgctgctaccatgatgatgatg

SEQ ID NO: 22 - DNA, 17 bp, Artificial Sequence
  Description of Artificial Sequence Synthetic oligonucleotide
cccttattccgatagtg

SEQ ID NO: 23 - DNA, 13 bp, Artificial Sequence
  Description of Artificial Sequence Synthetic oligonucleotide
cactatcggaata

SEQ ID NO: 24 - DNA, 32 bp, Artificial Sequence
  Description of Artificial Sequence Synthetic oligonucleotide
cccgaagatctcgatcccgcgaaattaatacg

SEQ ID NO: 25 - DNA, 38 bp, Artificial Sequence
  Description of Artificial Sequence Synthetic oligonucleotide
ctcctttgctagccataagggaattctccttcttaaag

SEQ ID NO: 26 - DNA, 33 bp, Artificial Sequence
  Description of Artificial Sequence Synthetic oligonucleotide
ggagccggtgagcgtgggtcacgcggtatcatt

SEQ ID NO: 27 - DNA, 31 bp, Artificial Sequence
  Description of Artificial Sequence Synthetic oligonucleotide
gacccacgctcaccggctccagatttatcag

SEQ ID NO: 28 - DNA, 33 bp, Artificial Sequence
  Description of Artificial Sequence Synthetic oligonucleotide
cgactcactatagggagacacacaacggtttcc

SEQ ID NO: 29 - DNA, 31 bp, Artificial Sequence
  Description of Artificial Sequence Synthetic oligonucleotide
gtctccctatagtgagtcgtattaatttcgc

SEQ ID NO: 30 - DNA, 20 bp, Artificial Sequence
  Description of Artificial Sequence Synthetic oligonucleotide
agtgccacctgacgtctaag

SEQ ID NO: 31 - DNA, 41 bp, Artificial Sequence
  Description of Artificial Sequence Synthetic oligonucleotide
gcggaattcggtctcaagggtcatcatcaccatcaccattg

SEQ ID NO: 32 - DNA, 31 bp, Artificial Sequence
  Description of Artificial Sequence Synthetic oligonucleotide
ccgcgaattcggtctcaagggtatctccttc

SEQ ID NO: 33 - DNA, 40 bp, Artificial Sequence
  Description of Artificial Sequence Synthetic oligonucleotide
atacccttgagaccgaattcggcaattcgcgcaattgcgg

SEQ ID NO: 34 - DNA, 25 bp, Artificial Sequence
  Description of Artificial Sequence Synthetic oligonucleotide
atggccgaccagctgactgaggagc

SEQ ID NO: 35 - DNA, 25 bp, Artificial Sequence
  Description of Artificial Sequence Synthetic oligonucleotide
cttggacaccagcacacggacaaac

SEQ ID NO: 36 - protein, 7 aa, Artificial Sequence
  Description of Artificial Sequence Synthetic peptide
HHHHHHG

SEQ ID NO: 37 - protein, 6 aa, Artificial Sequence
  Description of Artificial Sequence Synthetic 6xHis tag
HHHHHH

SEQ ID NO: 38 - DNA, 4221 bp, Artificial Sequence
  Description of Artificial Sequence Synthetic polynucleotide
taatacgactcactatagggagaccacaacggtttccctctagaaataattttgtttaactttaagaaggagatataccatgtctggttctcatcatcatcatcatcatggtagcagcggcgaaaacctgtattttcagtcccttagggcaggtacggagcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacaatcggtctgcagcagcacctgctctaagggtgatccggctgctaacaaagcccgaaaggaagctgagttggctgctgccaccgctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatctcgatcccgcgaaat

SEQ ID NO: 39 - DNA, 2744 bp, Artificial Sequence
  Description of Artificial Sequence Synthetic polynucleotide
agggtgatccggctgctaacaaagcccgaaaggaagctgagttggctgctgccaccgctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatctcgatcccgcgaaattaatacgactcactatagggagaccacaacggtttccctctagaaataattttgtttaactttaagaaggagatataccatgtctggttctcatcatcatcatcatcatggtagcagcggcgaaaacctgtattttcagtccctt

SEQ ID NO: 40 - protein, 8 aa, Artificial Sequence
  Description of Artificial Sequence Synthetic peptide
KGHHHHHH

SEQ ID NO: 41 - DNA, 4218 bp, Artificial Sequence
  Description of Artificial Sequence Synthetic polynucleotide
taatacgactcactatagggagaccacaacggtttccctctagaaataattttgtttaactttaagaaggagatataccatgtctggttctcatcatcatcatcatcatagcagcggcgaaaacctgtattttcagtcccttagggcaggtacggagcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacaatcggtctgcagcagcacctgctctaagggtgatccggctgctaacaaagcccgaaaggaagctgagttggctgctgccaccgctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatctcgatcccgcgaaat

SEQ ID NO: 42 - DNA, 2702 bp, Artificial Sequence
  Description of Artificial Sequence Synthetic polynucleotide
ggatctcgatcccgcgaaattaatacgactcactatagggagaccacaacggtttccctctagaaataattttgtttaactttaagaaggaattgcccttaagggcaattcgaagcttgaaggtaagcctatccctaaccctctcctcggtctcgattctacgcgtaccggtcatcatcaccatcaccattgagtttaaactatatagaataaaagaagaaaccttagctgagcaataactagcataaccccttggggcctctaaacgggtcttgaggggttttttgctgaaaggaggaactatatccggattaacgcttacaatttaggtggcacttttcggggaaatgtgcgcggaacccctatttgtttatttttctaaatacattcaaatatgtatccgctcatgagacaataaccctgataaatgcttcaataatgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagagtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaacgcgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagacgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgca

SEQ ID NO: 43 - DNA, 2702 bp, Artificial Sequence; identical to SEQ ID NO: 42
  Description of Artificial Sequence Synthetic polynucleotide

SEQ ID NO: 44 - DNA, 2708 bp, Artificial Sequence
  Description of Artificial Sequence Synthetic polynucleotide
agatctcgatcccgcgaaattaatacgactcactatagggagacacacaacggtttccctctagaaataattttgtttaactttaagaaggagatacccttttgagaccgaattcggtctcaaggggcatcatcaccatcaccattgagtttaaactatatagaataaaagaagaaaccttagctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcacgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccg

SEQ ID NO: 45 - DNA, 2723 bp, Artificial Sequence
  Description of Artificial Sequence Synthetic polynucleotide
agatctcgatcccgcgaaattaatacgactcactatagggagacacacaacggtttccctctagaaataattttgtttaactttaagaaggagatacccttgagaccgaattgccgaattcgcgcaattcggtctcaaggggcatcatcaccatcaccattgagtttaaactatatagaataaaagaagaaaccttagctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcacgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccg

SEQ ID NO: 46 - DNA, 2684 bp, Artificial Sequence
  Description of Artificial Sequence Synthetic polynucleotide
agggtcatcatcaccatcaccattgagtttaaactatatagaataaaagaagaaaccttagctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcacgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatctcgatcccgcgaaattaatacgactcactatagggagacacacgacggtttccctctagaaataattttgtttaactttaagaaggagataccctt

SEQ ID NO: 47 - DNA, 2714 bp, Artificial Sequence
  Description of Artificial Sequence Synthetic polynucleotide
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcgcccggggatcctctagagtcgacctgcagcccacattcgccattcaggctacgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattcgagctcgcccggggatcctctagagtcgacctgcagcccactgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 48 - protein, 6 aa, Artificial Sequence
  Description of Artificial Sequence Synthetic peptide
CCXXCC

SEQ ID NO: 49 - DNA, 81 bp, Artificial Sequence
  Description of Artificial Sequence Synthetic oligonucleotide
aggagatataccatgtctggttctcatcatcatcatcatcatggtagcagcggcgaaaaccctgtattttcagtcccttag

SEQ ID NO: 50 - protein, 23 aa, Artificial Sequence
  Description of Artificial Sequence Synthetic peptide
MSGSHHHHHHGSSGENLYFQSLM

SEQ ID NO: 51 - DNA, 119 bp, Artificial Sequence
  Description of Artificial Sequence Synthetic polynucleotide
taatacgactcactatagggagaccacaacggtttccctctagaaataattttgtttaactttaagaaggagatacccttatgnnnnnnnnnaaggggcatcatcaccatcaccattag